SEQUENCE LISTING (61 sequences)

SEQ ID NO: 1 - protein, 4 aa, Artificial Sequence
  A synthetic polypeptide
KDEL

SEQ ID NO: 2 - protein, 5 aa, Artificial Sequence
  A synthetic polypeptide
KFERQ

SEQ ID NO: 3 - protein, 4 aa, Artificial Sequence
  A synthetic polypeptide
QREK

SEQ ID NO: 4 - protein, 25 aa, Artificial Sequence
  A synthetic polypeptide
MAISGVPVLGFFIIAVLMSAQESWA

SEQ ID NO: 5 - protein, 676 aa, Zaire ebolavirus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTAVSNRAKNISGQSPARTSSDPGTNTTTEDHKIMASENSSAMVQVHSQGREAAVSHLTTLATISTSPQSPTTKPGPDNSTHNTPVYKLDISEATQVEQHHRRTDNDSTASDTPPATTAAGPPKAENTNTSKSTDLLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 6 - protein, 676 aa, Sudan ebolavirus
MEGLSLLQLPRDKFRKSSFFVWVIILFQKAFSMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKINNNTFVLLDRPHTPQFLFQLNDTIHLHQQLSNTTGKLIWTLDANINADIGEWAFWENKKNLSEQLRGEELSFETLSLNETEDDDATSSRTTKGRISDRATRKYSDLVPKDSPGMVSLHVPEGETTLPSQNSTEGRRVDVNTQETITETTATIIGTNGNNMQISTIGTGLSSSQILSSSPTMAPSPETQTSTTYTPKLPVMTTEEPTTPPRNSPGSTTEAPTLTTPENITTAVKTVLPQESTSNGLITSTVTGILGSLGLRKRSRRQVNTRATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLC

SEQ ID NO: 7 - protein, 681 aa, Marburg marburgvirus
MKTTCLLISLILIQGVKTLPILEIASNIQPQNVDSVCSGTLQKTEDVHLMGFTLSGQKVADSPLEASKRWAFRAGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGTLQEYNSTKNQTCAPSKKPLPLPTAHPEVKLTSTSTDATKLNTTDPNSDDEDLTTSGSGSGEQEPYTTSDAATKQGLSSTMPPTPSPQPSTPQQGGNNTNHSQGVVTEPGKTNTTAQPSMPPHNTTTISTNNTSKHNLSTPSVPIQNATNYNTQSTAPENEQTSAPSKTTLLPTENPTTAKSTNSTKSPTTTVPNTTNKYSTSPSPTPNSTAQHLVYFRRKRNILWREGDMFPFLDGLINAPIDFDPVPNTKTIFDESSSSGASAEEDQHASPNISLTLSYFPKVNENTAHSGENENDCDAELRIWSVQEDDLAAGLSWIPFFGPGIEGLYTAGLIKNQNNLVCRLRRLANQTAKSLELLLRVTTEERTFSLINRHAIDFLLARWGGTCKVLGPDCCIGIEDLSRNISEQIDQIKKDEQKEGTGWGLGGKWWTSDWGVLTNLGILLLLSIAVLIALSCICRIFTKYIG

SEQ ID NO: 8 - protein, 681 aa, Marburg marburgvirus
MKTIYFLISLILIQSIKTLPVLEIASNSQPQDVDSVCSGTLQKTEDVHLMGFTLSGQKVADSPLEASKRWAFRTGVPPKNVEYTEGEEAKTCYNISVTDPSGKSLLLDPPSNIRDYPKCKTVHHIQGQNPHAQGIALHLWGAFFLYDRVASTTMYRGKVFTEGNIAAMIVNKTVHRMIFSRQGQGYRHMNLTSTNKYWTSSNETRRNDTGCFGILQEYNSTNNQTCSPSLKPPSLPTVTPSIHSTNTQINTAKSGTMNPSSDDEDLMISGSGSGEQGPHTTLNVVTEQKQSSTILSTPSLHPSTSQHEQNSTNPSRHAVTEHNGTDPTTQPATLLNNTNTTPTYNTLKYNLSTPSPPTRNITNNDTQRELAESEQTNAQLNTTLDPTENPTTAQDTNSTTNIIMTTSDITSKHPTNSSPDSSPTTRPPIYFRKKRSIFWKEGDIFPFLDGLINTEIDFDPIPNTETIFDESPSFNTSTNEEQHTPPNISLTFSYFPDKNGDTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSKNISEQIDKIRKDEQKEETGWGLGGKWWTSDWGVLTNLGILLLLSIAVLIALSCICRIFTKYIG

SEQ ID NO: 9 - protein, 681 aa, Marburg marburgvirus
MRTTCFFISLILIQGIKTLPILEIASNDQPQNVDSVCSGTLQKTEDVHLMGFTLSGQKVADSPLEASKRWAFRTGVPPKNVEYTEGEEAKTCYNISVTDPSGKSLLLDPPTNVRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGTLQEYNSTKNQTCAPSKTPPPPPTARPEIKPTSTPTDATRLNTTNPNSDDEDLTTSGSGSGEQEPYTTSDAVTKQGLSSTMPPTPSPQPGTPQQGGNNTNHSQDAATELDNTNTTAQPPTPSHNTTTISTNNTSKHNLSTLSEPPQNTTNPNTQSMATENEKTSAPPKTTLPPTESPTTEKSTNNTKSPTTMEPNTTNGHFTSPSSTPNSTTQHLIYFRRKRSILWREGDMFPFLDGLINAPIDFDPVPNTKTIFDESSSSGASAEEDQHASSNISLTLSYLPHTSENTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSRNISEQIDQIKKDEQKEGTGWGLGGKWWTSDWGVLTNLGILLLLSIAVLIALSCICRIFTKYIG

SEQ ID NO: 10 - protein, 681 aa, Marburg marburgvirus
MKTTCLFISLILIQGIKTLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGRVFTEGNIAAMIVNKTVHKMIFSRQGQGYRHMNLTSTNKYWTSNNGTQTNDTGCFGALQEYNSTKNQTCAPSKIPSPLPTARPEIKPTSTPTDATKLNTTDPNSDDEDLATSGSGSGEQEPHTTSDAVTKQGLSSTMPPTPSPQPSTPQQEGNNTDHSQDAVTEPNKTNTTAQPSMPPHNTTAISTNNTSKHNFSTLSAPLQNTTNYDTQSTATENEQTSAPSKTTLPPTGNLTTAKSTNNTKGPTTTAPNMTNGHLTSPSPTPNPTTQHLVYFRKKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSRNISEQIDQIKKDEQKEGTGWGLGGKWWTSDWGVLTNLGILLLLSIAVLIALSCICRIFTKYIG

SEQ ID NO: 11 - DNA, 20 bp, Artificial Sequence
  A synthetic oligonucleotide
actctggtaactagagatcc

SEQ ID NO: 12 - DNA, 19 bp, Artificial Sequence
  A synthetic oligonucleotide
aattttgactagcggaggc

SEQ ID NO: 13 - DNA, 18 bp, Artificial Sequence
  A synthetic oligonucleotide
agatgggtgcgagagcgt

SEQ ID NO: 14 - DNA, 18 bp, Artificial Sequence
  A synthetic oligonucleotide
agatgggtgcgagaccgt

SEQ ID NO: 15 - DNA, 20 bp, Artificial Sequence
  A synthetic oligonucleotide
gctcctgtatctaatagagc

SEQ ID NO: 16 - DNA, 20 bp, Artificial Sequence
  A synthetic oligonucleotide
gctcctgtatctaataaagc

SEQ ID NO: 17 - DNA, 20 bp, Artificial Sequence
  A synthetic oligonucleotide
gctcctgtatctaacagagc

SEQ ID NO: 18 - DNA, 20 bp, Artificial Sequence
  A synthetic oligonucleotide
tttggtttccatcttcctgg

SEQ ID NO: 19 - DNA, 20 bp, Artificial Sequence
  A synthetic oligonucleotide
tttggtttccatcttcctgc

SEQ ID NO: 20 - DNA, 20 bp, Artificial Sequence
  A synthetic oligonucleotide
tttggcttccatctccctgg

SEQ ID NO: 21 - DNA, 20 bp, Artificial Sequence
  A synthetic oligonucleotide
tttggtttccatttccctgg

SEQ ID NO: 22 - DNA, 20 bp, Artificial Sequence
  A synthetic oligonucleotide
tttggtttccattttcctgg

SEQ ID NO: 23 - DNA, 20 bp, Artificial Sequence
  A synthetic oligonucleotide
gcaggacataacaaggtagg

SEQ ID NO: 24 - DNA, 20 bp, Artificial Sequence
  A synthetic oligonucleotide
gcaggacataacaaagtaga

SEQ ID NO: 25 - DNA, 20 bp, Artificial Sequence
  A synthetic oligonucleotide
aagataaagccacctttgcc

SEQ ID NO: 26 - DNA, 22 bp, Artificial Sequence
  A synthetic oligonucleotide
ttcttcctgccataggagatgc

SEQ ID NO: 27 - DNA, 22 bp, Artificial Sequence
  A synthetic oligonucleotide
ttcttcctgccataggaaatgc

SEQ ID NO: 28 - DNA, 20 bp, Artificial Sequence
  A synthetic oligonucleotide
gcagttgtaggctgacttcc

SEQ ID NO: 29 - DNA, 20 bp, Artificial Sequence
  A synthetic oligonucleotide
gcagttgtaggctgactccc

SEQ ID NO: 30 - DNA, 20 bp, Artificial Sequence
  A synthetic oligonucleotide
gcagttgtaggctggcttcc

SEQ ID NO: 31 - DNA, 20 bp, Artificial Sequence
  A synthetic oligonucleotide
gggatttggggttgctctgg

SEQ ID NO: 32 - DNA, 20 bp, Artificial Sequence
  A synthetic oligonucleotide
gggatttggggctgctctgg

SEQ ID NO: 33 - DNA, 22 bp, Artificial Sequence
  A synthetic oligonucleotide
tgatagtaggaggcttggtagg

SEQ ID NO: 34 - DNA, 22 bp, Artificial Sequence
  A synthetic oligonucleotide
tgatagtaggaggcttaatagg

SEQ ID NO: 35 - DNA, 22 bp, Artificial Sequence
  A synthetic oligonucleotide
tgatagtaggaggcttgatagg

SEQ ID NO: 36 - DNA, 20 bp, Artificial Sequence
  A synthetic oligonucleotide
gttaggcagggatattcacc

SEQ ID NO: 37 - DNA, 20 bp, Artificial Sequence
  A synthetic oligonucleotide
gttaggcagggatactcacc

SEQ ID NO: 38 - DNA, 20 bp, Artificial Sequence
  A synthetic oligonucleotide
ccctgtcttattcttctagg

SEQ ID NO: 39 - DNA, 20 bp, Artificial Sequence
  A synthetic oligonucleotide
ccctgtcttattcttacagg

SEQ ID NO: 40 - DNA, 20 bp, Artificial Sequence
  A synthetic oligonucleotide
ccctgtcttattcttgtagg

SEQ ID NO: 41 - DNA, 19 bp, Artificial Sequence
  A synthetic oligonucleotide
tagctgaggggacagatag

SEQ ID NO: 42 - DNA, 19 bp, Artificial Sequence
  A synthetic oligonucleotide
tagctgagggaacagatag

SEQ ID NO: 43 - DNA, 19 bp, Artificial Sequence
  A synthetic oligonucleotide
tagctggctggacagatag

SEQ ID NO: 44 - DNA, 23 bp, Artificial Sequence
  A synthetic oligonucleotide
atgggtggcaagtggtcaaaaag

SEQ ID NO: 45 - DNA, 23 bp, Artificial Sequence
  A synthetic oligonucleotide
atgggtggcaagtggtcaaaacg

SEQ ID NO: 46 - DNA, 23 bp, Artificial Sequence
  A synthetic oligonucleotide
atgggtggcaaatggtcaaaaag

SEQ ID NO: 47 - DNA, 23 bp, Artificial Sequence
  A synthetic oligonucleotide
atgggtggcaagtggtcaaaagg

SEQ ID NO: 48 - DNA, 18 bp, Artificial Sequence
  A synthetic oligonucleotide
ccagtacaggcaaaaagc

SEQ ID NO: 49 - DNA, 17 bp, Artificial Sequence
  A synthetic oligonucleotide
cagtacaggcgaaaagc

SEQ ID NO: 50 - DNA, 17 bp, Artificial Sequence
  A synthetic oligonucleotide
cagtacaggcaagaagc

SEQ ID NO: 51 - DNA, 51 bp, Artificial Sequence
  A synthetic oligonucleotide
taatacgactcactatagggagaccaccatgggtggcaagtggtcaaaaag

SEQ ID NO: 52 - DNA, 51 bp, Artificial Sequence
  A synthetic oligonucleotide
taatacgactcactatagggagaccaccatgggtggcaagtggtcaaaacg

SEQ ID NO: 53 - DNA, 51 bp, Artificial Sequence
  A synthetic oligonucleotide
taatacgactcactatagggagaccaccatgggtggcaagtggtcaaaagg

SEQ ID NO: 54 - DNA, 51 bp, Artificial Sequence
  A synthetic oligonucleotide
taatacgactcactatagggagaccaccatgggtggcaagtggtcaaaaat

SEQ ID NO: 55 - DNA, 82 bp, Artificial Sequence
  A synthetic oligonucleotide
ttttttttttttttttttttttttttttttttttttttttttttttttttttttttttttttttccagtacagggaaaaagc

SEQ ID NO: 56 - DNA, 81 bp, Artificial Sequence
  A synthetic oligonucleotide
ttttttttttttttttttttttttttttttttttttttttttttttttttttttttttttttttcagtacaggcgaaaagc

SEQ ID NO: 57 - DNA, 82 bp, Artificial Sequence
  A synthetic oligonucleotide
ttttttttttttttttttttttttttttttttttttttttttttttttttttttttttttttttccagtacaggcaagaagc

SEQ ID NO: 58 - DNA, 20 bp, Artificial Sequence
  A synthetic oligonucleotide
ccagaaagttctgccaggag

SEQ ID NO: 59 - DNA, 20 bp, Artificial Sequence
  A synthetic oligonucleotide
gccagctgtgtctcattgaa

SEQ ID NO: 60 - DNA, 18 bp, Artificial Sequence
  A synthetic oligonucleotide
tgccctgaggcactcttc

SEQ ID NO: 61 - DNA, 18 bp, Artificial Sequence
  A synthetic oligonucleotide
gtgccagggcagtgatct

(19) United States
(12) Patent Application Publication
(10) Pub. No.: US 2016/0160178 A1
(43) Pub. Date: Jun. 9, 2016

(54) IMMUNOTHERAPY USING STEM CELLS

(71) Applicant: White Oak Industries Inc., Archdale, NC (US)

(72) Inventors: Anthony T. Harrelson, Sophia, NC (US); Matt Kaufmann, Greensboro, NC (US)

(21) Appl. No.: 14/957,004

(22) Filed: Dec. 2, 2015

Related U.S. Application Data
(63) Continuation-in-part of application No. 14/632,440, filed on Feb. 26, 2015.
(60) Provisional application No. 62/185,443, filed on Jun. 26, 2015.
(60) Provisional application No. 62/098,780, filed on Dec. 31, 2014.
(60) Provisional application No. 62/089,661, filed on Dec. 9, 2014.

(51) Int. Cl. C12N 5/0784; C12N 7/00; A61M 1/36; A61K 39/285; A61K 39/07; A61K 39/12; A61K 39/21

(52) U.S. Cl. CPC C12N 5/0639; A61K 39/12; C12N 7/00; A61K 39/21; A61K 39/285; A61K 39/07; A61M 1/369; A61K 2039/5154

(57) ABSTRACT

Provided herein are cells, compositions and a method to produce in vitro programmed immune stimulating cells comprising: a) obtaining adipose cells from a subject; b) expanding said cells from step a) in culture so as to yield one or more populations of stem cells; c) differentiating said stem cells from step b) into hematopoetic stem cells, mesenchymal stem cells, connective cells, endothelial cells, cardio cells, osteo cells, muscle, cells and/or soft muscle tissue cells; and d) exposing said differentiated cells from step c) to 3D/4D printed molds of preselected bacterial, virus or particles thereof or exposing said differentiated cells from step c) to bacterial, virus or particles thereof so as to generate immune response triggering surface antigen expression in said differentiated cells from step c), thereby yielding in vitro programmed immune stimulating cells.

### RELATED APPLICATION

This application is a continuation-in-part and claims the benefit of priority from U.S. patent application Ser. No. 14/632,440, filed Feb. 26, 2015. This application also claims the benefit of priority from U.S. Provisional Application Ser. No. 62/089,661 filed Dec. 9, 2014; U.S. Provisional Application Ser. No. 62/098,780, filed on Dec. 31, 2014; and U.S. Provisional Application Ser. No. 62/185,443, filed on Jun. 26, 2015, each of which are incorporated in the entireties by reference.

### BACKGROUND

Immunotherapy, also called biologic therapy or biotherapy, is the treatment of disease by inducing, enhancing, or suppressing an immune response. In other words, immunotherapy is treatment that uses certain parts of a patient's immune system to fight diseases. Immunotherapies designed to elicit or amplify an immune response are classified as activation immunotherapies. This can be done in a couple of ways, for example, by stimulating a patient's own immune system to work harder or smarter to attack disease or by giving the immune system components, such as man-made immune system proteins (materials either made by the body or in a laboratory to improve, target, or restore immune system function). Immunotherapies that reduce or suppress are classified as suppression immunotherapies.

The active agents of immunotherapy are collectively called immunomodulators. They are a diverse array of recombinant, synthetic and natural preparations, including by not limited to cytokines, antibodies, vaccines, non-specific immunotherapies, granulocyte colony-stimulating factor (G-CSF), interferons, imiquimod, cellular membrane fractions from bacteria, IL-2, IL-7, IL-12, various chemokines, synthetic cytosine phosphate-guanosine (CpG) oligodeoxynucleotides and glucans. Immunomodulatory regimens offer an attractive approach as they often have fewer side effects than existing drugs, including less potential for creating resistance in microbial diseases.

Vaccines generally are considered to be one of the most efficient and cost-effective means of preventing infectious disease. However, vaccine approaches have thus far failed to provide protection against HIV, tuberculosis, malaria and many other diseases, including dengue, herpes and even the common cold.

### SUMMARY

The present inventors have recognized, among other things, that a problem to be solved can include improving the quality of treatment of a pathogen beyond that provided by pharmacological intervention, such as immunotherapy (e.g., application of medicaments). The present inventor has recognized, among other things, that a problem to be solved can include providing a supplemental or standalone form of treatment including hyperthermic treatment (heating) of blood carrying a pathogen and pathogen infected cells. Such pathogens include, but are not limited to, Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Hep A, Hep B, Hep C, the rhino virus, polio, and chickenpox virus, or HIV.

As discussed herein, in one example a hyperthermic treatment system diverts blood flow from the affected patient for instance by providing communication across the femoral arteries. The inflow of blood from one of the femoral arteries is moved through the system by a pump, for instance a peristaltic pump (e.g., a roller pump) or the like. The inflowing blood is delivered through a heat exchanger and correspondingly heated to a target treatment temperature configured to trigger pathogen and infected cell death. The target treatment temperature is determined based on the measurement of the present viral load of the pathogen of interest in the blood and associating the measurement with a corresponding treatment temperature (e.g., found in a standalone table, database for a controller or the like). In one example, higher temperatures are used with higher viral loads and relatively lower temperatures are used with lower viral loads. The blood is heated to the target temperature and pathogen infected cells and the pathogen suffer cell death and are captured in a filter. The filtered and heated blood is then cooled, for instance actively cooled with a second heat exchanger, to a temperature near body temperature, prior to returning the blood to the patient (e.g., to the second femoral artery).

The system and method described above enhances the effectiveness of immunotherapy treatments. For instance, by treating the patient with medicaments or the like beforehand the viral load of the patient is decreased prior to use of the hyperthermic treatment system. The hyperthermic treatment system provides a second treatment mechanism that further decreases the viral load of the patient. In another example, hyperthermic treatment is conducted as a first treatment mechanism and immunotherapy is used as a second treatment. In still another example, hyperthermic treatment is used as a standalone treatment to decrease viral load.

Another embodiment provides an immunogenic composition comprising in vitro matured stem or other cells, such as dendritic cells, and optionally comprising a pharmaceutically acceptable carrier. For example, in one embodiment, the dendritic cells are matured by contact with an immunogen from a pathogen, such as a virus. In another embodiment, the immunogen is a viral RNA. In one embodiment, the virus is Marburg virus, Ebola, Hantavirus, H5N1 strain of bird flu, Lassa virus, Junin virus, Dengue fever, Crimea-Congo fever virus, Bolivian hemorrhagic fever, HIV, Hep A, Hep B, Hep C, the rhino virus, polio, and chickenpox virus, or Kyasanur Forest Virus (KFD).

One embodiment provides a method to treat a pathogenic infection, such as a viral infection, comprising administering an effective amount of the immunogenic composition described herein to a subject in need thereof so as to treat said infection. For example, a method for treating or preventing an infection caused by a pathogenic species is described. The method comprises administering to a subject a therapeutic or prophylactic dosage of the cells as described herein. In one embodiment, the cell, such as dendritic cells, are autologous to said subject. Another embodiment provides for hyperthermia treatment before and/or after the immunotherapy treatment.

Another embodiment provides an immunogenic composition comprising in vitro matured stem or other cells, such as dendritic cells, and optionally comprising a pharmaceutically acceptable carrier. For example, in one embodiment, the dendritic cells are matured by contact with an immunogen from a pathogen, such as a virus. In another embodiment, the immunogen is a viral RNA. In one embodiment, the virus is Marburg virus, Ebola, Hantavirus, H5N1 strain of bird flu, Lassa virus, Junin virus, Dengue fever, Crimea-Congo fever virus, Bolivian hemorrhagic fever, HIV, Hep A, Hep B, Hep C, the rhino virus, polio, and chickenpox virus, or Kyasanur Forest Virus (KFD).

One embodiment provides a method to treat a pathogenic infection, such as a viral infection, comprising administering an effective amount of the immunogenic composition described herein to a subject in need thereof so as to treat said infection. For example, a method for treating or preventing an infection caused by a pathogenic species is described. The method comprises administering to a subject a therapeutic or prophylactic dosage of the cells as described herein. In one embodiment, the cell, such as dendritic cells, are autologous to said subject.

Another embodiment provides a method to produce in vitro matured stem cells, such as dendritic cells, comprising isolating dendritic cells from a subject, expanding said dendritic cells in vitro and contacting said cells with RNA from virus that has infected said subject so as to produce mature dendritic cells. In one embodiment, the virus is Marburg virus, Ebola, Hantavirus, H5N1 strain of bird flu, Lassa virus, Junin virus, Dengue fever, Crimea-Congo fever virus, Bolivian hemorrhagic fever, HIV, Hep A, Hep B, Hep C, the rhino virus, polio, and chickenpox virus or Kyasanur Forest Virus (KFD).

One embodiment provides a method to produce in vitro programmed immune cells comprising: a) obtaining stem cells from a subject; b) expanding said stem cells from step a) in culture; c) differentiating said stem cells from step b) into immune cells including T-cells, dendritic cells, or NK cells; and d) exposing said differentiated cells from step c) to 3D/4D printed molds of desired virus or virus particles so as to generate immune response triggering surface antigen expression in said differentiated cells from step c), thereby yielding in vitro programmed immune cells. In one embodiment, the immune cells are T-cells, such as CD⁺ T cells. In one embodiment, the stem cells are obtained from a human. In another embodiment, the stem cells are obtained from blood. One embodiment provides a method to treat a viral or bacterial infection comprising administering an effective amount of the in vitro programmed immune cells produced by said method. In one embodiment, the virus is Marburg virus, Ebola, Hantavirus, H5N1 strain of bird flu, Lassa virus, Junin virus, Dengue fever, Crimea-Congo fever virus, Bolivian hemorrhagic fever, HIV, Hep A, Hep B, Hep C, the rhino virus, polio, and chickenpox virus or Kyasanur Forest Virus (KFD).

The harvesting of stem cells will be conducted in a manner targeting various percentages of stem cells in such a way as to most efficiently treat the target virus. For example, Ebola, being a viral entity mostly inhibiting hemotological physiological function, can be treated with a cocktail of stem cells as follows: about 40-50% hematopoetic stem cells, about 30-40% mesenchymal stem cells, about 10-30% connective tissue, endothelial, cardio, induced pluripotent, osteo, muscle, and/or soft muscle tissue stem cells. The cocktail of stem cells will allow for tissue penetration and viral detection/inhibition/treatment. The reason for the combination of cells is so that the cell can be targeted to different tissues.

HIV and Ebola cell transduction vectors are particularly useful because of their ability to be pseudotyped to infect non-dividing hematopoietic stem cells (CD34⁺). This is done by transducing the packaging cell line used to package the vector with a nucleic acid which encodes the vesicular stomatitis virus (VSV) envelope glycoprotein, which is then expressed on the surface of the HIV vector. VSV infects CD34⁺ cells, and pseudotype HIV-2 vectors expressing VSV envelope proteins are competent to transduce these cells. CD34⁺ cells are important target cells for ex vivo gene therapy, because these cells differentiate into many different cell types, and because the cells are capable of re-engraftment into a patient undergoing ex vivo therapy. Stem cells differentiate in vivo into a variety of immune cells, including CD8⁺ cells which are the primary targets for HIV and Ebola infection.

Development of the immunotherapy element is unique in the aspect that the cells are cultured and expanded by lineage, i.e., hematopoietic stem cells will be cultured and expanded in the presence of hematopoietic stem cells, mesenchymal stem cells will be cultured and expanded in the presence of mesenchymal stem cells, connective, endothelial, cardio, induced pluripotent, osteo, muscle, and soft muscle stem cells will be cultured and expanded in the presence of their own cell type respectively. Once a proper population of viable cells is obtained, each cell type will be exposed to dissusable, tactile, mechanical, and electrical signals in such a combination as to induce cellular differentiation along desired lineage pathways available to each stem cell class. The groupings of stem cells will be passaged in such a manner as to preserve the existence of the stem cell as well as allowing for genesis is of a population with the intended purpose of undergoing differentiation mechanisms. Differentiation will be conducted in groupings so as to promote the intercellular differentiation benefits. Each differentiated cell class will be cultured and expanded in isolation of the other cell classes in the same fashion as the stem cell populations were.

Once the desired cell type is obtained in vitro, the cells will be exposed to the desired virus and will thus be programmed for the immunotherapy application. A small sampling of each cell grouping will be isolated and observed for viability as well as the expression of desired cell surface viral antigen markers. The protocol for the culturing, expansion, and differentiation will be repeated until a percentage of greater than 70% of cells are found viable and express the desired markers relevant for the targeted virus. Once all cell populations have reached the 70% viability mark and express viral antigen markers, the cell populations possessing the immunotherapy agents will be combined into a serum (for administration).

A virus mold can be generated by a 3D/4D printer used in the generation of each specific serum. For example, human DNA and cell structures including viruses can be designed on a computer and turned into 3D printable cell structures. These cell structures are recreated using bioprinting technology. Specifically, these printers take digital files and turn them into synthetic viruses, and the 3D printer turns STL files into objects. These models of the desired virus may be used to create personalized medicines that can affordably fight diseases that are, for example, still lethal or very difficult to cure. Medicines can be more effective when designed for a single person.

3D printing technology to carrier this out is available to an art worker. Digital files of DNA can be booted to become virus particles. Using 3D printers DNA synthesizer 3D printer, these digital files can then be synthetically printed as very specific, viruses. This opens up vaccines, diagnostics, gene therapies and other genetic materials. Diseases, like Ebola, can be attacked in a similar way (synthesis protocol). A mold can be manufactured to generate these antigens.

One embodiment provides for an immunogenic composition (stimulates an immune response) comprising the mixture cells disclosed herein and a pharmaceutically acceptable carrier. One embodiment provides for a method to treat a viral infection comprising administering to a subject in need thereof an effective amount of the cells produced by the methods described herein. In one embodiment, the administered cells are autologous to the subject. In one embodiment, the cells are administered parenterally, such as intravenously. In another embodiment, the cells are administered hyperthermically (heated).

With regards to RNA isolation, a virus sample is taken before the administration of any anti-viral (such as anti-retroviral) treatment. The surfaces of the manipulated dendritic cells (those exposed to virus and/or viral RNA obtained from, for example, the infected patient to be treated) present an increased number of, for example, HIV proteins, which allows them to stimulate the cytotoxic response of a certain type of immune cell called CD8⁺ lymphocytes.

RNA-loaded antigen-presenting cell (APC): the method involves introducing into an APC in vitro (i) derived RNA that includes virus-specific RNA which encodes a cell-surface viral antigenic epitope which induces T cell proliferation or (ii) pathogen-derived RNA that includes pathogen-specific RNA which encodes a pathogen antigenic epitope that induces T cell proliferation. Upon introducing RNA into an APC (i.e., “loading” the APC with RNA), the RNA is translated within the APC, and the resulting protein is processed by the MHC class I or class II processing and presentation pathways. Presentation of RNA-encoded peptides begins the chain of events in which the immune system mounts a response to the presented peptides.

One embodiment provides a method to produce in vitro programmed immune stimulating cells comprising: a) obtaining adipose cells from a subject; b) expanding said cells from step a) in culture so as to yield one or more populations of stem cells; c) differentiating said stem cells from step b) into hematopoetic stem cells, mesenchymal stem cells, connective cells, endothelial cells, cardio cells, osteo cells, muscle, cells and/or soft muscle tissue cells; and d) exposing said differentiated cells from step c) to 3D/4D printed molds of preselected bacterial, virus or particles thereof or exposing said differentiated cells from step c) to bacterial, virus or particles thereof so as to generate immune response triggering surface antigen expression in said differentiated cells from step c), thereby yielding in vitro programmed immune stimulating cells. In one embodiment, the subject is human. In one embodiment, the virus or particle thereof is selected from the group consisting of: adenoviruses; papillomaviruses; hepadnaviruses; parvoviruses; pox viruses; Epstein-Barr virus; cytomegalovirus (CMV); herpes simplex viruses; roseolovirus; varicella zoster virus; filoviruses; paramyxoviruses; orthomyxoviruses; rhabdoviruses; arenaviruses; coronaviruses; human enteroviruses; hepatitis A virus; human rhinoviruses; polio virus; retroviruses; rotaviruses; flaviviruses; hepaciviruses; and rubella virus. In another embodiment, the bacteria or particle thereof is selected from the group consisting of: Bacillus; Bordetella; Borrelia; Brucella; Burkholderia; Campylobacter; Chlamydia, Chlamydophila; Clostridium; Corynebacterium; Enterococcus; Escherichia; Francisella; Haemophilus; Helicobacter, Legionella; Leptospira; Listeria; Mycobacterium; Mycoplasma; Neisseria; Pseudomonas; Rickettsia; Salmonella; Shigella; Staphylococcus; Streptococcus; Treponema; Vibrio; and Yersinia. In one embodiment, the virus or particle is selected from the group consisting of: Marburg virus, Ebola, Hantavirus, H5N1 strain of bird flu, Lassa virus, Junin virus, Dengue fever, Crimea-Congo fever virus, Bolivian hemorrhagic fever, HIV, Hep A, Hep B, Hep C, the rhino virus, polio, and chickenpox virus or Kyasanur Forest Virus (KFD).

One embodiment provides a method to produce in vitro programmed immunmodulating cells comprising: a) obtaining hematopoetic, mesenchymal, connective tissue, endothelial, cardio, induced pluripotent, osteo, muscle, and/or soft muscle tissue stem cells from a subject; b) expanding said stem cells from a) in culture; c) contacting said stem cells obtained in b) with a pathogen or particles thereof, RNA or DNA coding for one or more proteins from bacterial or viral origin to expressed said protein or exposing said cells obtained in b) to desired virus, bacteria or particles thereof; and d) optionally combining said cells from step c) so as to comprise a mixture of cells. In one embodiment, the hematopeitic stem cells are differentiated to antigen presenting cells. In another embodiment, the antigen presenting cells are dendritic cells.

In one embodiment, the mixture of cells is from about 40-50% hematopoetic stem cells, about 30-40% mesenchymal stem cells, and about 10-30% connective tissue, endothelial, cardio, induced pluripotent, osteo, muscle, and/or soft muscle tissue stem cells. In one embodiment, the virus or particle thereof is selected from the group consisting of: adenoviruses; papillomaviruses; hepadnaviruses; parvoviruses; pox viruses; Epstein-Barr virus; cytomegalovirus (CMV); herpes simplex viruses; roseolovirus; varicella zoster virus; filoviruses; paramyxoviruses; orthomyxoviruses; rhabdoviruses; arenaviruses; coronaviruses; human enteroviruses; hepatitis A virus; human rhinoviruses; polio virus; retroviruses; rotaviruses; flaviviruses; hepaciviruses; and rubella virus. In one embodiment, the bacteria or particle thereof is selected from the group consisting of: Bacillus; Bordetella; Borrelia; Brucella; Burkholderia; Campylobacter; Chlamydia, Chlamydophila; Clostridium; Corynebacterium; Enterococcus; Escherichia; Francisella; Haemophilus; Helicobacter, Legionella; Leptospira; Listeria; Mycobacterium; Mycoplasma; Neisseria; Pseudomonas; Rickettsia; Salmonella; Shigella; Staphylococcus; Streptococcus; Treponema; Vibrio; and Yersinia. In one embodiment, the virus or particle thereof is selected from the group consisting of: Marburg virus, Ebola, Hantavirus, H5N1 strain of bird flu, Lassa virus, Junin virus, Dengue fever, Crimea-Congo fever virus, Bolivian hemorrhagic fever, HIV, Hep A, Hep B, Hep C, the rhino virus, polio, and chickenpox virus or Kyasanur Forest Virus (KFD).

One embodiment provides a method to treat or prevent a bacterial or viral infection comprising administering to a subject in need thereof an effective amount of the in vitro programmed immune stimulating cells produced by the methods described herein. In one embodiment, the administered cells are autologous to the subject. In another the embodiment, the cells are administered parenterally. In one embodiment, the subject further undergoes hyperthermic treatment.

One embodiment provides a method to treat or prevent a bacterial or viral infection comprising administering to a subject in need thereof an effective amount of the in vitro programmed immunmodulating cells produced by the methods described herein. One embodiment provides that the administered cells are autologous to the subject. In one embodiment, the cells are administered parenterally, such as intravenously. In another embodiment, the subject further undergoes hyperthermic treatment.

This summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic view of a hyperthermic treatment system.

FIG. 2 is a schematic view of one example of a hyperthermic treatment system including at least a hyperthermic heat exchanger.

FIG. 3 is a perspective view of one example of a hyperthermic heat exchanger configured to decrease viral load in a body fluid.

FIGS. 4A-B is a perspective view of one example of a perfusate filter configured to filter dead pathogen cells and dead pathogen infected cells for the body fluid.

FIG. 5 is a perspective view of one example of a cooling heat exchanger configured to cool the body fluid before return to a body.

FIG. 6 is a block diagram showing one example of hyperthermic treatment of a body fluid t no decrease viral load.

FIG. 7 is a block diagram showing another example of hyperthermic treatment of a body fluid to decrease viral load.

FIG. 8 panes 1-4 depict a method of assessing CD8 T cell antiviral function in acute infection. A CD8⁺ virus inhibition drawing (CD8 VIA) (1) and an HIV-1 peptide stimulation and soluble factor inhibition drawing (HIV-1-specific CD8 transwell VIA) (2). The other two drawings are for cell surface marker phenotype and intracellular cytokines from HIV-1 peptide stimulated primary CD8⁺ T lymphocytes using multiparameter intracellular cytokine staining (3) and antigen specific cytokine secretion in cell culture supernatants using multiplex cytokine measurements (4). The combination of the four provides a comprehensive measurement of CD8⁺ T cell antiviral function against both autologous and circulating HIV-1 isolates.

FIGS. 9A-D are a schematic of blood dendritic cell isolation. Magnetic labeling of monocytes and B cells (A). Monocytes and B cells are magnetically labeled using Non-Dendritic Cell Depletion Cocktail. Depletion of monocytes and B cells (B). Monocytes and B cells are retained in a magnetic field of the cell separator. The pre-enriched target cell fraction is collected in the flow-through. Magnetic labeling of dendritic cells (C). Blood dendritic cells are magnetically labeled using the Dendritic Cell Enrichment Cocktail. Positive selection of dendritic cells (D). Magnetically labeled dendritic cells are retained in the column. The column is removed from the magnetic field of the separator and blood dendritic cells are eluted.

FIG. 10 provides steps for a method for collecting, culturing, multiplying (expanding) and differentiating, in vitro, stem cells and using such cells for treatment, such as autologous treatment, of patients.

### DETAILED DESCRIPTION

### I. Hyperthermia

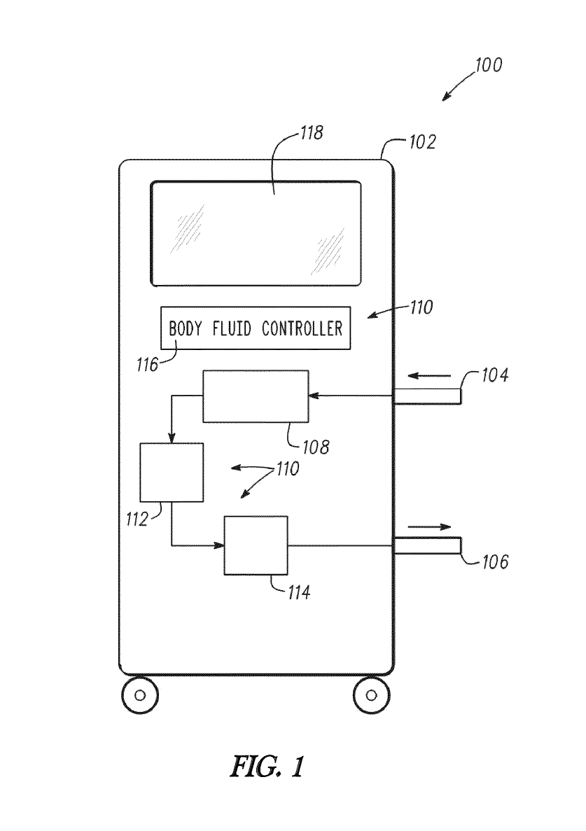

FIG. 1 shows one example of hyperthermic treatment system 100. The example hyperthermic treatment system 100 includes a system housing 102 including the components of a hyperthermic treatment assembly 110 therein. The hyperthermic treatment system 100 diverts a body fluid, such as blood, therethrough and treats the body fluid for one or more pathogens within the body fluid. For instance, as shown in FIG. 1 a body fluid inlet 104 (e.g., configured for communication with a femoral artery) extends into the system housing 102 into a circuit of components including components of the hyperthermic treatment assembly 110. After treatment of the body fluid the body fluid is returned to the body, for instance to an opposed femoral artery, through a body fluid outlet 106. The hyperthermic treatment system 100 decreases the viral load (quantity of pathogen) of the body fluid through the controlled application of heat to the body fluid to trigger cell death of the pathogen infected cells. In one example, and as described herein the hyperthermic treatment assembly 110 heats the body fluid to a target treatment temperature (based on the detected viral load of the body fluid) to trigger the death of the pathogen and pathogen infected cells in the body fluid. In another example, the body fluid is cooled to a return temperature after treatment, for instance with a cooling heat exchanger prior to return of the body fluid through the body fluid outlet 106.

The hyperthermic treatment system 100 and hyperthermic treatment assembly 110 are used alone or in combination with another form of immunotherapy (e.g., medicaments or the like) to decrease the viral load of a pathogen in the body fluid, such as the blood of an animal. Pathogens treated with the hyperthermic treatment system 100 and the hyperthermic treatment assembly 110 include, but are not limited to, Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Hep A, Hep B, Hep C, the rhino virus, polio, and chickenpox virus and HIV. The system 100 and assembly 110 are not limited to these pathogens and are instead applicable with any pathogen that suffers cell death with the elevation of body fluid temperature.

As shown in FIG. 1, the hyperthermic treatment system 100 includes a circuit of components within the system housing 102. The body fluid inlet 104 and body fluid outlet 106 in the hyperthermic treatment system 100 are configured to divert a flow of the body fluid from an animal body (animal as used herein includes any sort of animal including humans, mammals, reptiles, other non-sentient animals or the like). The body fluid is delivered through the body fluid inlet 104 and, after treatment by the hyperthermic treatment assembly 110, is returned to the body, for instance through the body fluid outlet 106. A circuit through the system housing 102 is configured to pump the body fluid therethrough and heat the body fluid with a hyperthermic heat exchanger to trigger the death of pathogen cells or pathogen infected cells in the body fluid prior to return through the body fluid outlet 106. The process performed by the hyperthermic treatment system 100 is optionally repeated in the manner of a bypass system that continuously or near continuously cycles the body fluid from the body through the system 100 and then returns the body fluid to the animal. Stated another way, the body fluid is continuously cycled from the body through the hyperthermic treatment system 100 according to a specified treatment time, detected instant viral load and viral load target or the like.

Referring again to FIG. 1, the hyperthermic treatment system 100 includes an input/output device 118 provided on the system housing 102. In one example, the input/output device 118 includes one or more diagnostic features for instance blood pressure monitors, flow rate monitors, oxygen content monitors (of the body fluid), temperature of the body fluid at various points in the hyperthermic treatment system 100 circuit and the like. In another example, the input/output device 118 includes one or more input features including, but not limited to, a touchscreen interface, keyboard, key pad or the like configured to input one or more values to the hyperthermic treatment system 100. For instance the input/output device 118 is used to input a measured viral load, pathogen type, animal (patient) information or the like into the system 100 for instance for use by a body fluid controller 116.

As described herein the viral load input of a particular pathogen to the input/output device 118 is used to determine a corresponding target treatment temperature for use with the hyperthermic heat exchanger 112. That is to say, the body fluid controller 116 optionally includes a database of a plurality of pathogens and viral load and target treatment temperature combinations for each of the pathogens. The body fluid controller, in an example, automatically determines a target treatment temperature based on the input viral load in combination with the identification of the pathogen to be treated. In still another example, the input/output device 118 is used to manually enter a target treatment temperature for use with the hyperthermic treatment assembly 110 to accordingly treat a pathogen.

The body fluid controller 116 shown in the system housing 102 is in communication with the input/output device 118 as well as a number of features within the hyperthermic treatment assembly 110 including the hyperthermic heat exchanger 112 and an optional cooling heat exchanger 114. The body fluid controller 116 receives one or more of a determined viral load and pathogen of a animal coupled with the body fluid inlet and outlet 104, 106. After inputting one or more of the pathogen as well as the animal viral load the body fluid controller 116 (e.g., using a database of known pathogens and viral load and target treatment temperature pairings) delivers a target treatment temperature to the hyperthermic heat exchanger 112 and controls the hyperthermic heat exchanger 112 to raise the temperature of the body fluid to the specified target treatment temperature. As discussed herein, the target treatment temperature triggers the death of a pathogen or pathogen infected cells in the body fluid while the body fluid is passed through the hyperthermic treatment system 100. In another example, the body fluid controller 116 cooperates with a plurality of temperature sensors, for instance in the circuit including the hyperthermic treatment assembly 110, to measure the temperature of the body fluid passing through the hyperthermic heat exchanger 112. The body fluid controller uses the temperature measures to control the hyperthermic heat exchanger 112 (and optionally the cooling heat exchanger 114) by way of feedback control to achieve the target treatment temperature. In a further example, the body fluid controller 116 uses temperature sensors inline in the circuit shown in FIG. 1 to measure the input and output temperatures of the body fluid flowing into and through the cooling heat exchanger 114 and accordingly controls the cooling heat exchanger 114 to achieve a return body fluid temperature similar to the body temperature of the animal coupled with the hyperthermic treatment system 100. That is to say, the body fluid controller 116 in an example uses a plurality of inline temperature sensors in the flow circuit in the system 100 and at one or more the inputs or outputs of features of the hyperthermic treatment assembly 110 (e.g., the hyperthermic heat exchanger 112 and the cooling heat exchanger 114) to accordingly raise and lower the body fluid temperature to achieve both death of the pathogen and pathogen infected cells and conditioning of the body fluid to a temperature nearer to the body fluid temperature prior to return to the body of the animal (through the body fluid outlet 106).

As further shown in FIG. 1, in one example the circuit of features within the hyperthermic treatment system 100 include a body fluid pump 108. The body fluid pump 108 provides the flow of body fluid from the body fluid inlet 104 into the body fluid outlet 106. The body fluid pump 108 is optionally in communication with the body fluid controller 116 (for instance it is controlled by the body fluid controller 116) to provide a target flow rate of the body fluid to each of the features of the hyperthermic treatment assembly 110 including one or more of the hyperthermic heat exchanger 112 and the cooling heat exchanger 114. That is to say, in another example the body fluid controller 116 controls both the flow rate and the temperature of the body fluid through the hyperthermic treatment system 100 to accordingly reach a desired target treatment temperature at the hyperthermic heat exchanger 112, a return temperature at the cooling heat exchanger 114, and to do so at a desired flow rate for instance a flow rate matching a body fluid removal flow rate through the body fluid inlet 104 and a desired body fluid return flow rate at the body fluid outlet 106.

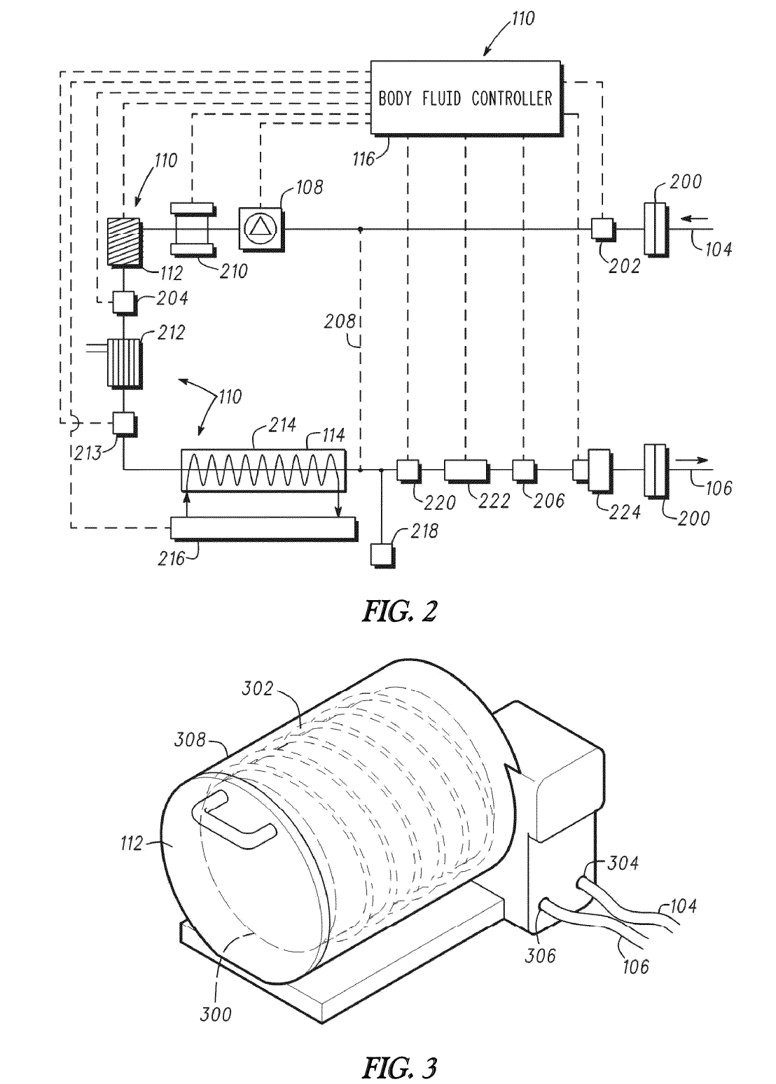

FIG. 2 shows a detailed schematic view of the example hyperthermic treatment assembly 110. In FIG. 2 the hyperthermic treatment assembly 110 is an inline series of components of an overall fluid flow circuit through the hyperthermic treatment system 100. The fluid flow circuit includes the body fluid inlet 104 (and body fluid out let 106) and one or more flow clamps 200. The body fluid inlet and outlet 104, 106 included one or more of ports, peripheral catheters or the like or interfaces configured to couple with the same to provide fluid communication between the body of an animal and the hyperthermic treatment system 100. The flow clamps 200 in one example provide a mechanical feature that engages with the body fluid inlet 104 (for instance a pliable tube) to close flow into the flow circuit of the hyperthermic treatment system 100.

Continuing along the flow circuit, a first temperature sensor 202 is provided after the body fluid inlet 104 and prior to the body fluid pump 108. In one example, the first temperature sensor 202 is positioned elsewhere within the flow circuit, for instance immediately prior to the hyperthermic heat exchanger 112. The first temperature sensor 202 cooperates with a second temperature sensor 204 downstream from the sensor 202 in another example to accordingly measure the input and output temperatures for the hyperthermic heat exchanger 112. As discussed herein, the input and output temperature measurements used by the body fluid controller 116 to adjustment the heating of the hyperthermic heat exchanger 112.

Optionally, a bypass line 208 is provided across the flow circuit of the hyperthermic treatment system 200 to accordingly provide an emergency bypass around the components of the hyperthermic treatment system. The bypass line 208 facilitates direct or near direct communication between the body fluid inlet 104 and the body fluid outlet 106.

As further shown in FIG. 2, the body fluid pump 108 is provided in the flow circuit of the hyperthermic treatment system 100. In the example shown in FIG. 2 the body fluid pump 108 is a rotary pump that provides a measured and controlled flow rate of the body fluid through the hyperthermic treatment system 100. In another example, the body fluid pump 108 includes any sort of metering pump configured to provide a precise flow of a body fluid through the system. For instance the body fluid pump 108 includes, but is not limited to, a rotary pump, peristaltic pump or the like.

In another example, a gas bubble sensor 210 is provided downstream from the body fluid pump 108. Optionally, the gas bubble sensor 210 is positioned in other portions of the flow circuit of the hyperthermic treatment system 100. For instance, the gas bubble sensor 210 is positioned downstream from any feature that may generate gas bubbles within the flow of the body fluid prior to the return to the body (e.g., through the body fluid outlet 106). In one example, the gas bubble sensor 210 includes one or a plurality of different sensor types configured to detect gas bubbles such as air bubbles within the body fluid flow. For instance, the gas bubble sensor includes, but is not limited to, an infrared sensor, a reflective light sensor, an ultrasound sensor or the like.

Referring again to FIG. 2, the hyperthermic heat exchanger 112 is provided in the flow circuit of the hyperthermic treatment system 100. In one example, the hyperthermic heat exchanger 112 includes a heating core and a body fluid conduit. In one example, the body fluid conduit wraps around the heating core to facilitate heat exchange between the heating core and the body fluid within the body fluid conduit. The hyperthermic heat exchanger 112 as described herein raises the temperature of the body fluid to a target treatment temperature configured to trigger the death of a pathogen or pathogen infected cells in the body fluid.

As will be further described herein, in one example the first temperature sensor 202 and the second temperature sensor 204 (corresponding to input and output temperature sensors for the hyperthermic heating exchanger 112) are used in combination by the body fluid controller 116 to automatically control the temperature of the hyperthermic heat exchanger 112. That is to say, the body fluid controller 116 achieves the target treatment temperature by measuring the first and second temperatures at the first and second temperature sensors 202, 204 and accordingly adjusts the heat applied from the heating core to the body fluid in the body fluid conduit to ensure elevation of the body fluid.

As further shown in FIG. 2, the hyperthermic treatment assembly 110 includes a filter 212. In one example, the filter 212 is an inline perfusate filter provided between the hyperthermic heat exchanger 112 and the cooling heat exchanger 114. As described further herein, the perfusate filter 212 includes a plurality of perforated flow channels or tubes. The perforated flow channels are exposed to a crossing flow of infusion fluid such as saline or the like that is delivered into a waste removal reservoir provided around the perforated flow channels. The saline entrains dead or dying pathogen cells and pathogen infected cells from the perforations. The entrained wastes are drawn with the infusion fluid from the filter 212. The remainder of the body fluid including healthy blood cells, white blood cells or the moves downstream towards the body fluid outlet 106 and other components of the hyperthermic treatment system 100.

As further shown in FIG. 2 the hyperthermic treatment assembly 110 includes an optional cooling heat exchanger 114. As discussed herein the hyperthermic heat exchanger 112 raises the body fluid temperature to a target treatment temperature. The cooling heat exchanger lowers the elevated body fluid temperature to avoid shocking the system of the animal. The cooling heat exchanger 114 provides active heat exchange to the body fluid prior to its reintroduction through the body fluid outlet 106. As shown in FIG. 2, the cooling heat exchanger 114 includes a heat exchange jacket 214 and a coolant conditioner 216. In one example the coolant conditioner 216 provides chilled water to the heat exchange jacket 214 (e.g., through a cooling coil within the heat exchange jacket 214) to cool the body fluid flowing through a center passage toward the body fluid outlet 106.

Optionally, the second temperature sensor 204 and a third temperature sensor 206 are used in combination by the body fluid controller 116 to measure the input and output temperatures of the body fluid into and out of the cooling heat exchanger 114 and facilitate the accurate control and cooling of the body fluid to a desired temperature. In one example, the body fluid controller 116 uses the measured input and output temperatures from the sensors 204, 206 to control the heat transfer at the cooling heat exchanger 114 and lower the body fluid temperature to a temperature equivalent to the body temperature of the animal coupled with the hyperthermic treatment system 100. Optionally, the cooling heat exchanger 114 is used to lower the body fluid temperature to a temperature just above the body temperature of the animal to allow for continued heat transfer from the body fluid to the environment (cooling) and reintroduction of the body fluid at the body temperature at the body fluid outlet 106.

As further shown in FIG. 2, in one example the hyperthermic treatment system 100 includes a fluid port 218 (e.g., hemostatis valve, touhy port, piercable diaphragm or the like). The fluid port 218 provides an injectable port for the application of immunotherapy, medicaments or the like to the flow of the body fluid through the hyperthermic treatment system 100. For instance, a drip line or dedicated immunotherapy injection system is provided at the fluid port 218. That is to say, in one example the hyperthermic treatment system 100 is used to administer one or more medicines or the like to the body fluid in addition to conditioning the body fluid with heat to a target treatment temperature. Optionally, the fluid port 218 facilitates the drawing of body fluid (e.g., by syringe) for diagnostic purposes including updating the measurement of the viral load of an animal coupled to the hyperthermic treatment system 100.

As further shown in FIG. 2 a flow sensor 220 and a flow controller 222 are provided in line with the flow circuit. In one example, the flow sensor 220 measures the flow of the body fluid through the hyperthermic treatment system 100 and cooperates with the flow controller 222 (a valve) to adjust the flow of the body fluid prior to return delivery through the body fluid outlet 106. The body fluid controller 116 is in communication with each of the flow sensor 220 and the flow controller 222 and monitors the flow of the body fluid through the hyperthermic treatment system 100 and ensures precise delivery of the body fluid to the animal with the flow controller 222. For instance, the body fluid controller 116 uses the flow sensor 220 and the flow controller 222 to match the flow rate of the body fluid at the body fluid outlet 106 to the flow rate through the body fluid inlet 104. Accordingly, the body fluid controller 116 in combination with the flow sensor 220 and the flow controller 222 monitors the flow rate of the body fluid through the hyperthermic treatment system 100 and ensures that the inflow of body fluid into the treatment system 100 is identical to or closely matches the outflow of the body fluid to the animal. In still another example, the body fluid pump 108 is used with the flow sensor 220 to control the flow rates into and out of the hyperthermic treatment system 100. Optionally, the body fluid pump and the flow controller 222 are used together to adjust the flow of the body fluid.

Referring again to FIG. 2, a body fluid warmer is optionally provided at the end of the flow circuit. The body fluid warmer 224 provides a heat exchanger immediately prior to the reintroduction of the body fluid to the animal. In one example, the body fluid warmer 224 works in cooperation with the third temperature sensor 206 (in communication with the body fluid controller 116) to adjust the heat transfer to and temperature of the body fluid immediately prior to exit at the body fluid outlet 106. That is to say, in one example the third temperature sensor 206 is also provided immediately adjacent to the body fluid warmer 224 near to the point of reintroduction of the body fluid to the patient, for instance through the body fluid outlet 106. The body fluid warmer 224 provides a final heat exchanger to ensure the returning body fluid is provided to the animal at a desired return temperature (e.g., closely matching the body temperature of the animal). In another example, the body fluid warmer 224 is optional. Stated another way, the hyperthermic heat exchanger 112 and the cooling heat exchanger 114 are operated in cooperation with the body fluid controller 116 and the plurality of temperature sensors (e.g., sensors 202, 204, 206) to both raise and lower the body fluid temperature to a respective target treatment temperature and target return temperature (e.g., body temperature).

Referring again to FIG. 2, the hyperthermic treatment assembly 110 includes the body fluid controller 110, the body fluid pump 108, the hyperthermic heat exchanger 112, the cooling heat exchanger 114 and the filter 212. As previously described herein, the body fluid pump 108 provides a measured flow of body fluid from the body fluid inlet 104 (coupled with a femoral artery) through the hyperthermic treatment assembly 110. The flow of body fluid is delivered through the hyperthermic heat exchanger 112 and accordingly raised to a target treatment temperature. In one example, the target treatment temperature is determined by assessing the viral load of the animal coupled with the hyperthermic treatment system 100. Upon a determination of the viral load as well as identification of the particular pathogen infecting the animal a target treatment temperature is determined, for instance by inputting the target treatment temperature or viral load and pathogen identifier to the body fluid controller 116 (e.g., through the input/output device 118 shown in FIG. 1).

In one example, the hyperthermic heat exchanger 112 in cooperation with a first upstream temperature sensor 202 and a second downstream temperature sensor 204, raises the temperature of the body fluid to the target treatment temperature. For instance, the body fluid controller 116 uses the input and output temperatures at the first and second temperature sensors 202, 204 to accordingly operate the hyperthermic heat exchanger 112 by heating a heating core of the heat exchanger to accordingly raise the temperature of the body fluid within the body fluid conduit of the hyperthermic heat exchanger.

After heating of the body fluid to the desired target treatment temperature the body fluid is delivered within the hyperthermic treatment assembly 110 to the filter 212. As previously described herein, in one example the filter 212 includes a plurality of perforated flow channels surrounded by a waste removal reservoir. A flow of liquid, for instance saline, is provided through the waste removal reservoir and interacts with the perforations of the perforated flow channels to entrain dead and dying pathogen cells and pathogen infected cells out of the flow of the body fluid. The remainder of the clean body fluid (e.g., healthy blood cells, white blood cells and the like) flows from the filter 212 toward the body fluid outlet 106.

In another example, the hyperthermic treatment assembly 110 includes a cooling heat exchanger 114 provided downstream from the filter 212 and upstream from the body fluid outlet 106. The flow of heated body fluid through the cooling heat exchanger 114 is conditioned by the cooling heat exchanger 114 to a temperature substantially equivalent to (matching or nearly matching) the body temperature of the animal coupled with the hyperthermic treatment system 100. For instance, the cooling conditioner 216 provides a flow of cool liquid (chilled water or the like) through a cooling coil within the heat exchange jacket 214. The flow of chilled water through the heat exchange jacket 214 cools the liquid within the heat exchange jacket 214 surrounding a center passage of the cooling heat exchanger 114 having the body fluid therein. The heat exchange jacket 214 and the cooling coil therein cool the body fluid to a desired return temperature. In one example, the second temperature sensor 204 and the third temperature sensor 206 are used as input and output temperature sensors for the cooling heat exchanger 114 by the body fluid controller 116. The body fluid controller 116 uses the input and output temperatures measured with the sensors 204, 206 to control cooling of the body fluid to a temperature matching or nearly matching that of the body prior to delivery through the body fluid outlet 106. As described herein, an optional body fluid warmer 224 is provided downstream within the hyperthermic treatment assembly 110 to ensure delivery of the body fluid to the animal at a desired return temperature prior to delivery through the body fluid outlet 106.

FIG. 3 shows one example of hyperthermic heat exchanger 112 for use with the hyperthermic treatment system 100 shown in FIGS. 1 and 2. In one example, the hyperthermic heat exchanger 112 includes a heating core 300 provided within a body fluid conduit 302. As shown in FIG. 3, the heating core 300 is provided centrally relative to the body fluid conduit 302, and the conduit is wrapped around the heating core 300 in a plurality of circuits in the manner of a coil. As further shown in FIG. 3, the hyperthermic heat exchanger 112 includes input and output ports 304, 306. The input port 304 is in communication with the body fluid inlet 104 (upstream) and the output port 306 is in communication with the body fluid outlet 106 (downstream). The components of the hyperthermic heat exchanger 112 are in one example housed within a heat exchanger body 308. Optionally, the heat exchanger body 308 and the other components of the hyperthermic heat exchanger 112 are housed within the system housing 102 shown in FIG. 1 for the hyperthermic treatment system 100.

As previously described herein the hyperthermic heat exchanger 112 is an inline component of a hyperthermic treatment assembly 110. The hyperthermic heat exchanger 112 raises the temperature of a body fluid, such as blood, to trigger death of a pathogen and pathogen infected cells of the body fluid. As previously described the body fluid controller 116 (see FIGS. 1 and 2) is in communication with the hyperthermic heat exchanger 112 and accordingly controls the heating of a body fluid directed through the body fluid conduit 302. For instance, in one example the heating core 300 is a resistive heating core and the body fluid controller 116 controls the heating of the heating core 300 by way of the voltage applied to the heating core to thereby generate corresponding heat to raise the temperature of body fluid within the body fluid conduit 302. In another example, the heating core 300 includes a heating coil therein. For instance a medium such as heated water, steam or the like is directed through the heating core 300 to heat the body fluid present and directed through the body fluid conduit 302 of the hyperthermic heat exchanger 112.

As previously described herein, in one example the body fluid controller 116 controls the hyperthermic heat exchanger 112 to heat the body fluid provided therein from a temperature approximating the body temperature of an animal to a higher temperature, for instance a target treatment temperature, to trigger death of pathogens and pathogen infected cells in the body fluid. Referring to FIG. 2, first and second temperature sensors 202, 204 provided near to the corresponding input port 304 and output port 306 of the hyperthermic heat exchanger 112 are optionally used in combination with the body fluid controller 116 to control the heating of the heating core 300 to achieve the target treatment temperature for the body fluid within the body fluid conduit 302. For instance, an input temperature is measured with the first temperature sensor 202 and an output temperature is measured with the second temperature sensor 204. The body fluid controller 116 uses these measurements in combination with a desired target treatment temperature (determined according to the particular pathogen in question as well as a viral load of the pathogen within the body fluid) to control the hyperthermic heat exchanger 112 (e.g., the heating of the heating core 300) to heat the body fluid to the desired target treatment temperature.

In another example the hyperthermic heat exchanger 112 is operated in a non-feedback configuration, for instance the performance of the heating core 300 is known (e.g., known voltages or currents correspond to known heating core temperatures) and the body fluid controller 116 provides a specified voltage, quantity of heating medium or the like to the heating core 300. The heating core 300 correspondingly raises its temperature to a temperature corresponding to the target treatment temperature and the body fluid within the body fluid conduit 302 is heated to the target treatment temperature.

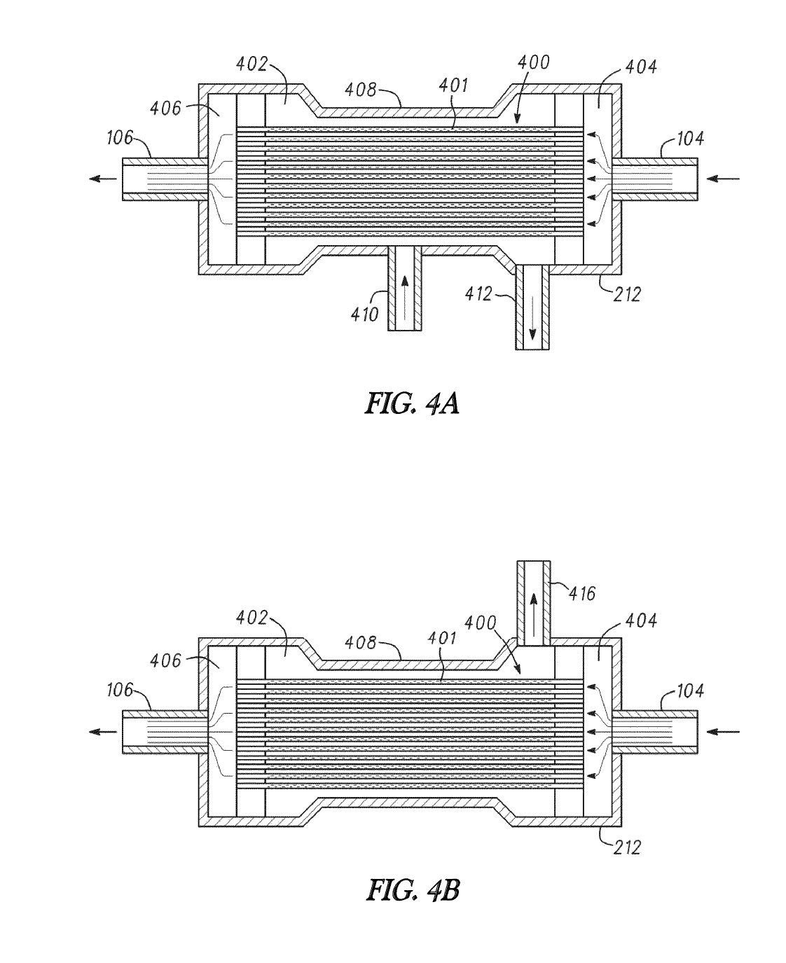

FIG. 4 shows one example of a filter 212 for use with the hyperthermic treatment system 100 shown in FIGS. 1 and 2. In one example the filter 212 is a perfusate filter including a plurality of perforations provided in one or more flow channels. Referring again to FIG. 4, the filter 212 includes a plurality of perforated flow channels 400 including perforations 401. The perforated flow channels 400 are provided within a reservoir jacket 408 that includes the waste removal reservoir 402. The waste removal reservoir 402 accordingly surrounds the plurality of perforated flow channels 400. As further shown in FIG. 4, in one example the body fluid inlet 104 is in communication with an inlet manifold 404 of the filter 212. In a similar manner an outlet manifold 406 of the filter 212 is in communication with the body fluid outlet 106 (the body fluid inlet 104 and outlet 106 are both shown in FIG. 2).

As further shown in FIG. 4 and previously discussed herein, the plurality of perforated flow channels 400 are provided within the waste removal reservoir 402. An infusion port 410 and an extraction port 412 are also in communication with the waste removal reservoir 402 and accordingly the perforations 401 along the perforated flow channels 400. The infusion port 410 provides a flow of an infusion fluid, such as saline, to the waste removal reservoir 402 that washes over the perforated flow channels 400 and the perforations 401 provided there along. The flow of solution across the perforated flow channels 400 entrains waste including dead and dying pathogens and pathogen infected cells having a size and configuration sufficient (e.g., sufficiently small) to fit through the perforations 401. The infusion port 410 and the extraction port 412 provide a continuous or near continuous flow of infusion fluid while body fluid is supplied from the inlet manifold 404 through the perforated flow channels 400 and to the outlet manifold 406. The delivery of the infusion fluid to the perforated flow channels 400 continuously entrains waste from the flowing body fluid through the perforations 401 (e.g., dead and dying pathogens and pathogen infected cells) and the waste is removed through the extraction port 412. In one example the perforations 401 are provided in the perforated flow channel 400 with one or more of a specified diameter or shape ensuring passage of dead and dying pathogens and pathogen infected cells but otherwise substantially preventing the passage of other components of the body fluid, for instance red blood cells, white blood cells and the like. Accordingly, the perforations 401 in combination with the infusion fluid provided through the infusion port 410 filter waste such as dead and dying pathogens and pathogen infected cells from the body fluid (death triggered by the hyperthermic heat exchanger 112) and thereby clean the body fluid prior to delivery to the animal through the body fluid outlet 106.

In another example, the hyperthermic treatment assembly 110 includes a body fluid composition sensor 213 (e.g., a blood composition sensor or blood leak sensor). The body fluid composition sensor 213 measures changes in the body fluid composition, such as the concentration of blood within the body fluid relative to other components (e.g., pathogens). The body fluid composition sensor 213 thereby provides an indication of the functionality of one or more of the preceding filter 212 or the hyperthermic heat exchanger 112. For instance, if the blood concentration is low a higher than expected concentration of pathogen may be present and the treatment temperature may correspondingly be below a temperature configured to trigger the desired pathogen death. The body fluid controller 116 is operated to increase the treatment temperature and accordingly increase the blood concentration (by decreasing the pathogen concentration) in the manner of feedback control. In another example, a lower blood concentration may indicate the filter 212 is not operating at a desired efficiency and service or an increased flow of saline is needed to increase the efficiency.

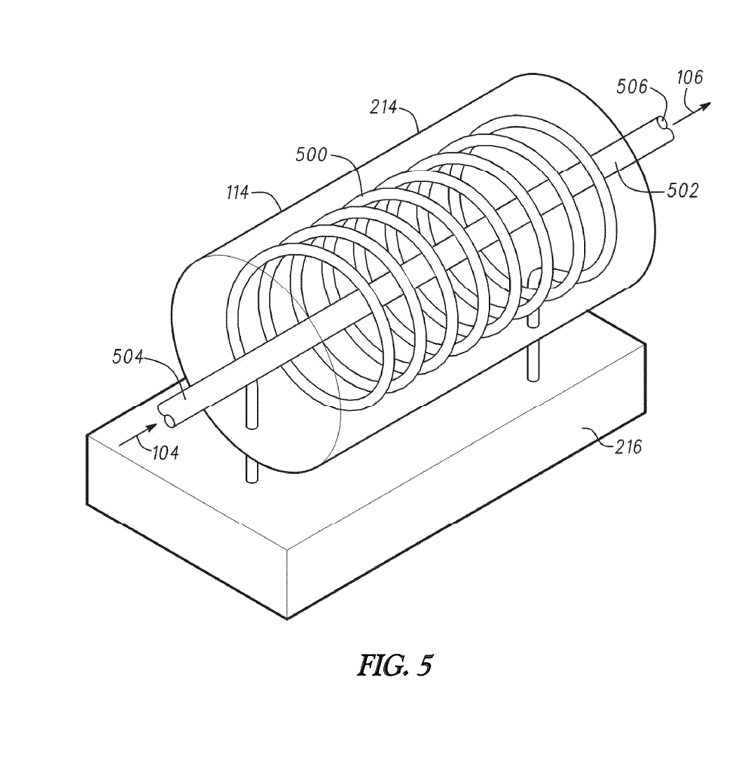

FIG. 5 shows one example of a cooling heat exchanger 114. As previously described herein in one example the cooling heat exchanger 114 is included as a component of the hyperthermic treatment assembly 110 shown in FIG. 2. Optionally, the cooling heat exchanger 114 is absent from the hyperthermic treatment assembly 110 for instance where the body fluid has a dwell time within the hyperthermic treatment assembly 110 sufficient to cool the body fluid prior to return delivery through a body fluid outlet 106 to an animal. Referring now to FIG. 5, the cooling heat exchanger 114 is shown with at least two components including a heat exchange jacket 214 and a coolant conditioner 216. In the example shown in FIG. 5 the heat exchange jacket 214 includes a cooling coil 500 extending through the heat exchange jacket 214. A body fluid conduit 502 for carrying the body fluid extends through the cooling coil 500 as shown. The body fluid conduit 502 is in communication with the body fluid inlet 104 and the body fluid outlet 106, for instance by way of corresponding input and output ports 504, 506. As further shown in FIG. 5, the cooling coil 500 and the body fluid conduit 502 are provided within the heat exchange jacket 214. In one example, the heat exchange jacket 214 includes a cavity therein to house the cooling coil 500 and the body fluid conduit 502 and bathe both of said components in a medium, such as liquid water.

The cooling coil 500 provides a flow of coolant therein such as refrigerant or chilled water to cool the body fluid within the body fluid conduit 502. For instance, the cooling coil 500 serves as a heat exchange component with the liquid within the heat exchange jacket 214. By cooling the liquid within the heating exchange jacket 214 (e.g., water) the body fluid within the body fluid conduit 502 is similarly cooled. That is to say, one or more of convective or conductive forms of heat transfer are provided between the cooling coil 500, the medium within the heat exchange jacket 214 and the body fluid conduit 502.

As further shown in FIG. 5, a coolant conditioner 216 is coupled with the cooling coil 500. The coolant conditioner 216 includes components for cooling a refrigerant or chilled water pumped through the cooling coil 500. In one example, the coolant conditioner 216 includes a refrigerant system including for chilling (and optionally changing the phase) of a refrigerant such as a compressor, a coil to facilitate heat transfer between the coolant and an exhaust medium such as air, an expansion valve, a pump and the like. In another example, the coolant conditioner 216 includes a cold water chiller including one or more features such as a miniature cooling tower or the like. As the coolant (chilled water or refrigerant) is conditioned at the coolant conditioner 216 it is pumped through the cooling coil 500 to accordingly cool the body fluid within the body fluid conduit 502 prior to delivery through the body fluid outlet 106 to return to the animal.

As previously described and shown in FIG. 2 the coolant conditioner 216 is in communication with the body fluid controller 116. In one example, the coolant conditioner 216, the second temperature sensor 204 and the third temperature sensor 206 are used in combination by the body fluid controller 116 to accordingly control the heat exchange between the body fluid and the coolant within the cooling coil 500 to thereby achieve a target return temperature prior to delivery of the body fluid through the body fluid outlet 106 for return to the animal. For example, the second temperature sensor 204 provides an input temperature of the body fluid into the cooling heat exchanger 114 and the third temperature sensor 206 provides an output temperature of the body fluid out of the cooling heat exchanger 114. The body fluid controller 116 uses these temperature inputs to control the operation of the coolant conditioner 216 (including for instance the coolant temperature, coolant flow rate and the like) to achieve a target return temperature equivalent to or slightly higher than a body temperature of the animal coupled with the hyperthermic treatment system 100 (to account for heat loss in the system 100).

As will be described herein, in one example a body fluid warmer 224 is also provided with the hyperthermic treatment system 100 to provide a final heat transfer feature for the body fluid immediately prior to delivery of the body fluid back to the animal. The body fluid warmer 224 is an optional component, and the hyperthermic heat exchanger 112 and the cooling heat exchanger 114 are used exclusively together in an example to condition the temperature of the body fluid (e.g., raise the temperature for pathogen death and lower the temperature for reintroduction).

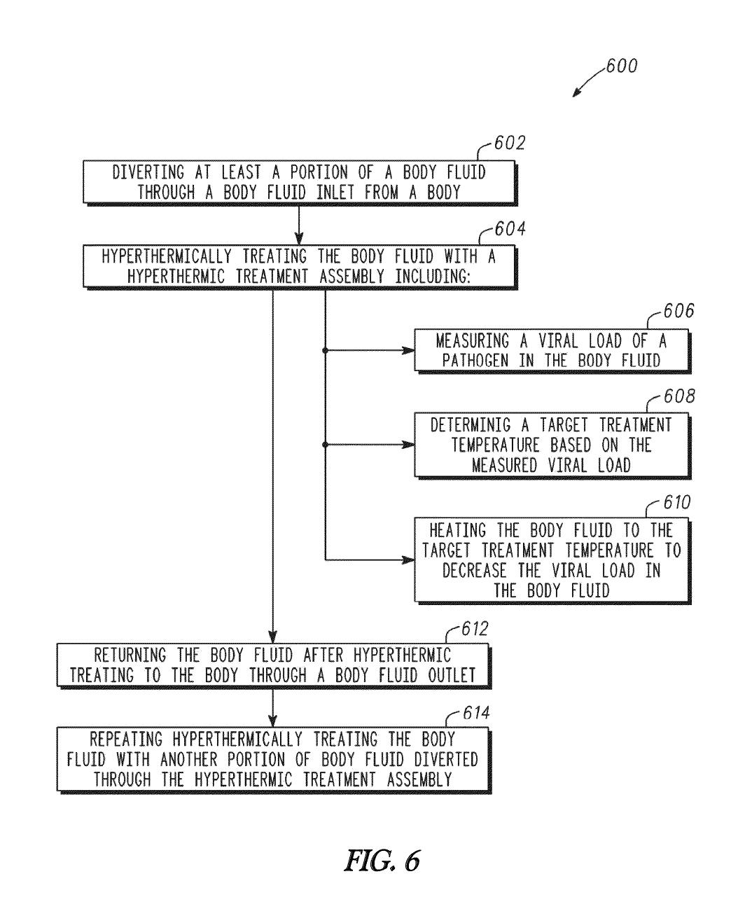

FIG. 6 shows one example of a method 600 to hyperthermically treat the body fluid of an animal, such as a human, mammal, reptile or other non-sentient animal. In describing the method 600 reference is made to one or more components, features, functions and steps previously described herein. Where convenient reference is made to the components, features, steps and the like with reference numerals. Reference numerals provided are exemplary and are not exclusive. For instance, components, features, functions, steps and the like described in the method 600 include, but are not limited to, the corresponding numbered elements provided herein other corresponding features described herein (both numbered and unnumbered) as well as their equivalents.

At 602, the method 600 includes diverting at least a portion of a body fluid through a body fluid inlet 104 from a body, for instance, a body of an animal including but not limited to a human, non-sentient animals or the like. In one example, the body fluid is delivered to a hyperthermic treatment system 100 including a hyperthermic treatment assembly 110 through a body fluid inlet 104. Optionally, the body fluid inlet 104 (as well as the body fluid outlet 106) includes a flow clamp 200 to selectively prevent and allow the flow of the body fluid into the hyperthermic treatment system 100.

At 604, the body fluid is hyperthermically treated with a hyperthermic treatment assembly 110. One example of a hyperthermic treatment assembly is shown in FIG. 2. As discussed herein, hyperthermically treating the body fluid includes raising the body fluid temperature to a target treatment temperature that will trigger cell death in the pathogen and pathogen infected cells. At 606 hyperthermically treating the body fluid includes measuring a viral load of a pathogen in the body fluid. In one example, prior to treatment of the body fluid a sample of the body fluid is taken to measure the viral load of a particular pathogen within the body fluid. In another example, the viral load of the animal is measured in an on-going manner by the hyperthermic treatment system 100. At 608, a target treatment temperature for the body fluid is determined based on the measured viral load. For instance, the body fluid controller 116 includes a database of target treatment temperatures paired with corresponding viral loads for a particular pathogen. In one example, for a particular pathogen a table is provided with increasing target treatment temperatures relative to increasing viral loads. Accordingly, with a higher measured viral load a higher target treatment temperature is identified for the hyperthermic treatment system 100.

At 610, the body fluid is heated to the target treatment temperature to decrease the viral load in the body fluid. In one example, a hyperthermic heat exchanger 112 (shown in FIG. 2) is provided in line with the hyperthermic treatment assembly 110. Optionally, the hyperthermic heat exchanger 112 is in communication with the body fluid controller 116. The body fluid controller 116 operates the hyperthermic heat exchanger 112, for instance by generating heat in a heating core 300 of the heat exchanger to correspondingly elevate the temperature of the body fluid in a body fluid conduit 302 and thereby trigger cell death in the pathogen and pathogen infected cells. In another example, heating the body fluid to the target treatment temperature includes using one or more sensors such as first and second temperature sensors 202, 204 to measure the input and output temperatures to the hyperthermic heat exchanger 112. The body fluid controller 116 uses the inputs from each of the sensors 202, 204 to correspondingly monitor and control the hyperthermic heat exchanger 112 to achieve the target treatment temperature in the body fluid flowing there through.

At 612, the body fluid is returned to the body after hyperthermic treatment. For instance, as shown in FIG. 2 a body fluid outlet 106 is provided in the hyperthermic treatment system 100 to facilitate the delivery of the treated body fluid back to the body of the animal. In one example, the body fluid inlet 104 is coupled with the body of the animal at a first femoral artery and the body fluid outlet is coupled at a second femoral artery.

At 614, the method 600 further includes repeating hyperthermically treating the body fluid with another portion of body fluid diverted through the hyperthermic treatment assembly 110. That is to say, in one example, the body fluid inlet 104 is coupled with the portion of the body and the body fluid outlet 106 is coupled with another portion of the body and the body fluid is cycled from the animal through the system 100 in an on-going manner. Accordingly, the hyperthermic assembly 110 is operated in a continuous fashion to facilitate the cycling of the body fluid from the animal through the treatment assembly 110 to repeatedly treat the body fluid with the hyperthermic heat exchanger 112. By conditioning the body fluid (elevating the temperature to a target treatment temperature) the viral load in the body fluid is gradually decreased as the entirety (including near entirety) of the body fluid is cycled through the hyperthermic treatment assembly 110.

Several options for the method 600 follow. In one example, repeating hyperthermically treating the body fluid includes repeating hyperthermically treating the body fluid until a target viral load is measured. For instance, in one example, the hyperthermic treatment system 100 includes an instrument in line with the hyperthermic treatment assembly 110 (e.g., coupled with the fluid port 208) configured to continuously or at intervals measure the viral load of the body fluid during treatment. Accordingly, treatment is continued and then ceased once a target viral load is reached. In another example, the viral load of the animal is determined with a standalone method, for instance, by drawing a body fluid from the animal either from the fluid port 218 or with a hypodermic syringe and testing the body fluid.

In another example, the method 600 further includes cooling the body fluid after heating to a return temperature (lower than the elevated target treatment temperature) prior to returning body fluid to the body, for instance, through the body fluid outlet 106. In an example, cooling the body fluid includes active heat exchange between the body fluid and a cooling heat exchanger, such as the cooling heat exchanger 114 shown in FIG. 2.

In still another example, heating the body fluid to the target temperature (for instance, discussed at 610) includes heating a heating core 300 of the hyperthermic heat exchanger 112 to a core temperature based on the target treatment temperature. In one example, the core temperature is higher than the target treatment temperature and accordingly accounts for heat transfer from the heating core 300 (shown in FIG. 3) to the surrounding environment and housing (the heat exchanger body 308). In still another example, heating the body fluid to the target temperature includes delivering the body fluid through a body fluid conduit 302 wrapped around the heating core 300. The heating core 300 provides conductive and convective heat transfer to the body fluid within the body fluid conduit 302 by heating the body fluid conduit 302.

In another example, the method 600 includes filtering the body fluid after heating the body fluid to the target temperature. Filtering includes removing dead or dying pathogens and dead or dying pathogen infected cells from the body fluid. One example of such a filter 212 is shown in FIG. 4. The filter 212 includes a plurality of perforated flow channels 400 having perforations 401 therein. The perforated flow channels 400 receive the body fluid after heating to the target treatment temperature. The body fluid flows through the perforated flow channels 400 and a flow of infusion fluid such as saline provided through an infusion port 410 to a waste removal reservoir 402 entrains the dead and dying pathogen cells and pathogen infected cells through the perforations 401. The filtered body fluid is then delivered out of the filter 212 (e.g., to an outlet manifold 406).

Optionally, returning the body fluid (for instance after filtering) includes measuring a flow rate of the body fluid with the flow sensor 220. In one example, the flow rate of the body fluid is controlled according to the measured flow rate. As previously described herein, in one example, the flow sensor 220 and the flow controller 222 (an adjustable valve) are in communication with the body fluid controller 116. The body fluid controller 116 controls the flow controller 222 according to the measured flow rate through the flow sensor 220. The body fluid controller 116 in combination with the flow sensor and controller 220, 222 ensures that the inflow into the hyperthermic treatment system 100 for instance through the body fluid inlet 104 matches the outflow of the system 100 through the body fluid outlet 106.

In still another example, the method 600 further includes treating one or more chronic infectious diseases. Optionally, the hyperthermic treatment of the body fluid comprises treating one or more of, but not only, the Marberg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus and HIV.

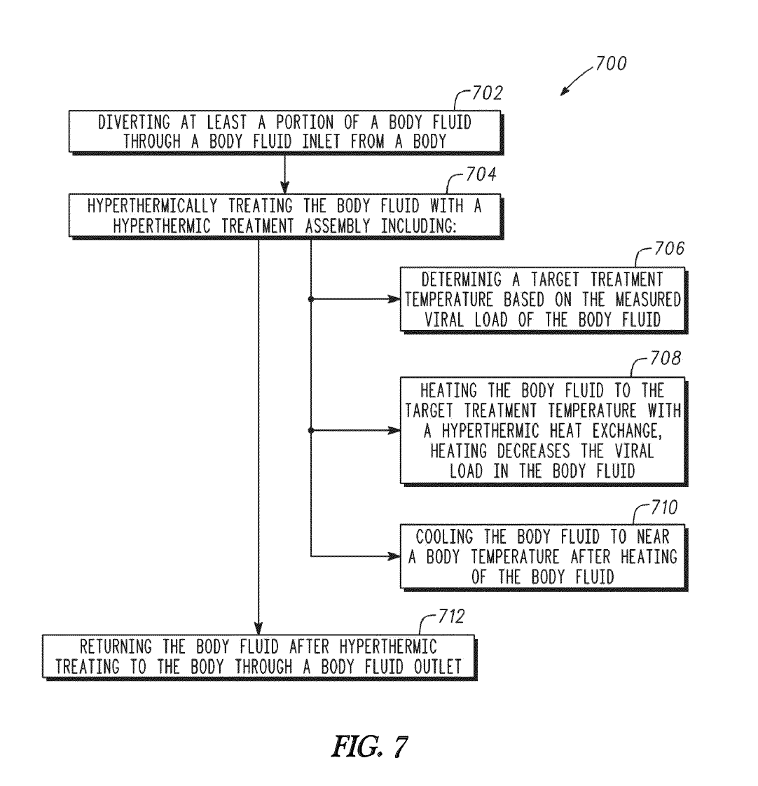

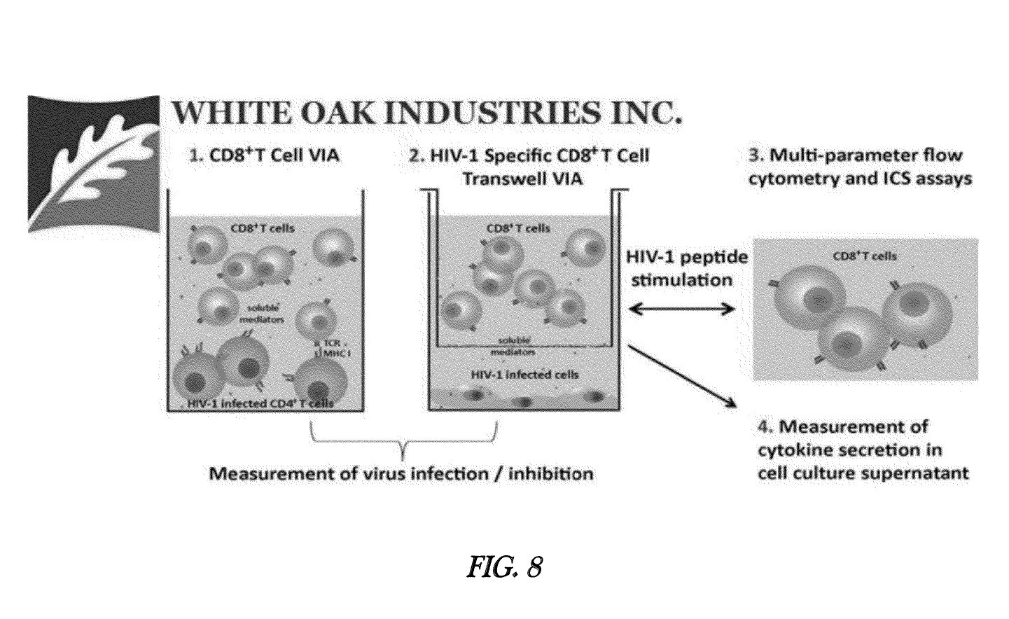

FIG. 7 shows one example of the method 700 for hyperthermically treating a body fluid of an animal. In describing the method 700 reference is made to one or more components, features, functions and steps previously described herein. Where convenient reference is made to the components, features, steps and the like with reference numerals. Reference numerals provided are exemplary and are not exclusive. For instance, components, features, functions, steps and the like described in the method 700 include but are not limited to the corresponding numbered elements provided herein, other corresponding features described herein both numbered and unnumbered as well as their equivalents.

At 702, the method 700 includes diverting at least a portion of a body fluid through a body fluid inlet 104 from a body. At 704, the body fluid is hyperthermically treated with the hyperthermic treatment assembly 110. In one example, the hyperthermic treatment assembly 110 shown in FIG. 2. As discussed herein, hyperthermically treating the body fluid includes elevating the temperature of the body fluid to a target treatment temperature that triggers cell death of the pathogen and pathogen infected cells. Hyperthermically treating the body fluid includes at 706 determining the target treatment temperature based on a measured viral load of the body fluid. As previously described herein, in one example a testing procedure is conducted for the animal to determine the viral load of a particular pathogen in the animal. The target treatment temperature is determined based on this viral load and the particular pathogen detected. For instance, the body fluid controller 116 includes a database including a plurality of target treatment temperatures associated with particular viral loads of a specified pathogen. Optionally, the database includes a catalogue of pathogens with corresponding viral loads and associated target treatment temperatures for each of those viral loads.

Hyperthermically treating the body fluid further includes at 708 heating the body fluid to the target treatment temperature with the hyperthermic heating exchanger 112. For instance, a heating core 300 is heated to accordingly heat a body fluid conduit 302 of the hyperthermic heat exchanger 112. Heating the body fluid decreases the viral load in the body based on the heat applied to the body fluid.

At 710, the body fluid is cooled to near a body temperature (matches the body temperature or is near to the body temperature) after heating of the body fluid and prior to returning the body fluid to the body, for instance through a body fluid outlet 106. As previously described herein, in one example, cooling the body fluid includes active heat exchange, for instance conducted by a cooling heat exchanger 104 provided downstream from the hyperthermic heat exchanger 112.

At 712, the method 700 includes returning the body fluid after hyperthermic treatment to the body through a body fluid outlet 106. As discussed herein, the returning body fluid has a decreased viral load as a function of the hyperthermic treatment, for instance with the hyperthermic heat exchanger 112. Additionally, the returning body fluid is provided to the body at a return temperature near to body temperature and lower than the target treatment temperature achieved with the hyperthermic heat exchanger 112.

Several options for the method 700 follow. In one example, the method 700 includes repeating hyperthermically treated the body fluid with another portion of body fluid diverted through the hyperthermic treatment assembly 110. As previously described herein, in one example, the body fluid inlet 104 is coupled with a femoral artery and the body fluid outlet 106 is coupled with an opposed femoral artery. Diverting at least a portion of the body fluid through a body fluid inlet from the body includes continuously diverting portions of the body fluid therethrough. Accordingly, the body fluid is cycled in a continuous or near continuous fashion through the hyperthermic treatment assembly 110 to condition all or a large portion of the body fluid of the animal to accordingly decrease the viral load throughout the volume of the body fluid.

In another example, hyperthermically treating the body fluid, for instance cooling the body fluid, includes measuring a first output temperature of the body fluid near an output of the hyperthermic heat exchanger 112 for instance the second temperature sensor 204 shown in FIG. 2 and measuring an output of the cooling heat exchanger 104 for instance at the third temperature sensor 206 also shown in FIG. 2. Cooling the body fluid to near body temperature after heating (with the hyperthermic heat exchanger 112) includes cooling according to the measured first and second output temperatures (measured at the respective sensors 204, 206). That is to say, in one example a controller such as the body fluid controller 116 of the hyperthermic treatment assembly 110 is used in cooperation with the second and third temperature sensors 204, 206 to control active heat exchange at the cooling heat exchanger 114 to thereby lower the temperature of the body fluid to a return temperature near that of the body.

In a similar manner, hyperthermically treating the body fluid (heating) includes measuring a first input temperature of the body fluid near an input of the hyperthermic heat exchanger 112, for instance with a first temperature sensor 202, and measuring a first output temperature of the body fluid near an output of the hyperthermic heat exchanger, for instance with the second temperature sensor 204. Heating the body fluid to the target treatment temperature includes heating to the target treatment temperature according to the measured first input and first output temperatures. That is to say, in another example the body fluid controller 116 uses the inputs from the first and second temperature sensors 202, 204 corresponding to input and output temperatures for the hyperthermic heat exchanger 112 to control the heating of the hyperthermic heat exchanger 102 (e.g., a heating core 300) to achieve the target treatment temperature accurately and precisely to ensure a decrease of the viral load to the desired degree.

In another example, the method 700 includes filtering the body fluid after heating the body fluid to the target treatment temperature. Filtering includes removing dead or dying pathogens and dead or dying pathogen infected cells from the body fluid. One example of such a filter 212 is shown in FIG. 4 and includes a plurality of perforated flow channels 400 and a cross-flow of infusion fluid provided by an infusion port 410 and drawn out of the filter 212 through an extraction port 412. The infusion fluid draws dead or dying pathogen cells and pathogen infected cells through the perforations 401 and entrains the same within the infusion fluid for eventual extraction through the extraction port 412.

In another example, the method 700 includes measuring a flow rate of the body fluid with the flow sensor 220 shown in FIG. 2 and controlling the flow rate of the body fluid according to the measured flow rate for instance with a flow controller 222 in communication with the body fluid controller 116.

In still another example the method 700 includes treating one or more chronic infectious diseases. Chronic infectious diseases treated with the hyperthermic treatment of body fluid (at 704) include, but are not limited to, Marberg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus and HIV.

### II. Immunotherapy

### One Embodiment Provides for Immunotherapy and Optionally with Hyperthermia (Discussed Above) as a Combined Treatment with the Immunotherapy

### DEFINITIONS

The term “antigen” is well understood in the art and includes substances which are immunogenic, i.e., immunogen. It will be appreciated that the use of any antigen is envisioned for use in the present invention and thus includes, but is not limited to a self-antigen (whether normal or disease-related), an infectious antigen (e.g., a microbial antigen, viral antigen, etc.), or some other foreign antigen (e.g., a food component, pollen, etc.). The term “antigen” or alternatively, “immunogen” may apply to collections of more than one immunogen, so that immune responses to multiple immunogens may be modulated simultaneously. Moreover, the term includes any of a variety of different formulations of immunogen or antigen.

A “native” or “natural” or “wild-type” antigen is a polypeptide, protein or a fragment which contains an epitope, which has been isolated from a natural biological source, and which can specifically bind to an antigen receptor, when presented as an MHC/peptide complex, in particular a T cell antigen receptor (TCR), in a subject.

The terms “major histocompatibility complex” or “MHC” refers to a complex of genes encoding cell-surface molecules that are required for antigen presentation to T cells and for rapid graft rejection. In humans, the MHC is also known as the “human leukocyte antigen” or “HLA” complex. The proteins encoded by the MHC are known as “MHC molecules” and are classified into Class I and Class II MHC molecules. Class I MHC molecules include membrane heterodimeric proteins made up of a chain encoded in the MHC non-covalently linked with the 2-microglobulin. Class I MHC molecules are expressed by nearly all nucleated cells and have been shown to function in antigen presentation to CD8⁺ T cells. Class I molecules include HLA-A, B, and C in humans. Class II MHC molecules also include membrane heterodimeric proteins consisting of non-covalently associated α and β chains. Class II MHC molecules are known to function in CD4⁺ T cells and, in humans, include HLA-DP, -DQ, and -DR.

The term “antigen presenting cells (APCs)” refers to a class of cells capable of presenting one or more antigens in the form of peptide-MHC complex recognizable by specific effector cells of the immune system, and thereby inducing an effective cellular immune response against the antigen or antigens being presented. APCs can be intact whole cells such as macrophages, B-cells, endothelial cells, activated T-cells, and dendritic cells; or other molecules, naturally occurring or synthetic, such as purified MHC Class I molecules complexed to β2-microglobulin. Many types of cells are capable of presenting antigens on their cell surface for T-cell recognition.

The term “dendritic cells (DCs)” refers to a diverse population of morphologically similar cell types found in a variety of lymphoid and non-lymphoid tissues, Steinman (1991) Ann. Rev. Immunol. 9:271-296. Dendritic cells constitute potent APCs in the organism. While the dendritic cells can be differentiated from monocytes or hematopoietic cells, they possess distinct phenotypes. For example, a particular differentiating marker, CD14 antigen, is not found in dendritic cells, but is possessed by monocytes. Also, mature dendritic cells are not phagocytic, whereas the monocytes are strongly phagocytosing cells. It has been shown that mature DCs can provide all the signals necessary for T cell activation and proliferation.

Dendritic cells can be stimulated to activate a cytotoxic response towards an antigen. Dendritic cells, a type of antigen presenting cell, are harvested from a patient. These cells are then either pulsed with an antigen or transfected with a viral vector. Upon transfusion back into the patient these activated cells present antigen to effector lymphocytes (CD4⁺ T cells, CD8⁺ T cells, and B cells). This initiates a cytotoxic response to occur against cells expressing the antigens (against which the adaptive response has now been primed).

Adoptive cell transfer uses T cell-based cytotoxic responses to attack cells. T cells that have a natural or genetically engineered reactivity to a patient's disease are generated in vitro and then transferred back into the patient.

The Autologous immune enhancement therapy (AIET) is an autologous immune cell based therapy wherein the patient's own peripheral blood-derived NK cells Cytotoxic T Lymphocytes and other relevant immune cells are expanded in vitro and then reinfused.

The term “immune effector cells” refers to cells capable of binding an antigen and which mediate an immune response. These cells include, but are not limited to, T cells, B cells, monocytes, macrophages, NK cells and cytotoxic T lymphocytes (CTLs), for example CTL lines, CTL clones, and CTLs from tumor, inflammatory, or other infiltrates.

A “naïve” immune effector cell is an immune effector cell that has never been exposed to an antigen capable of activating that cell. Activation of naïve immune effector cells requires both recognition of the peptide:MHC complex and the simultaneous delivery of a costimulatory signal by a professional APC in order to proliferate and differentiate into antigen-specific armed effector T cells.

“Immune response” broadly refers to the antigen-specific responses of lymphocytes to foreign substances. Any substance that can elicit an immune response is said to be “immunogenic” and is referred to as an “immunogen”. All immunogens are antigens, however, not all antigens are immunogenic. An immune response of this invention can be humoral (via antibody activity) or cell-mediated (via, for example, T cell activation).

As used herein, the term “educated, antigen-specific immune effector cell”, is an immune effector cell as defined above, which has previously encountered an antigen. In contrast to its naïve counterpart, activation of an educated, antigen specific immune effector cell does not require a costimulatory signal. Recognition of the peptide:MHC complex is sufficient.

“Activated”, when used in reference to a T cell, implies that the cell is no longer in GO phase, and begins to produce one or more of cytotoxins, cytokines and other related membrane-associated proteins characteristic of the cell type (e.g., CD8⁺ or CD4⁺), and is capable of recognizing and binding any target cell that displays the particular peptide/MHC complex on its surface, and releasing its effector molecules.

As used herein, the term “inducing an immune response in a subject” is a term understood in the art and refers to an increase of at least about 2-fold, or alternatively at least about 5-fold, or alternatively at least about 10-fold, or alternatively at least about 100-fold, or alternatively at least about 500-fold, or alternatively at least about 1000-fold or more in an immune response to an antigen (or epitope) which can be detected or measured, after introducing the antigen (or epitope) into the subject, relative to the immune response (if any) before introduction of the antigen (or epitope) into the subject. An immune response to an antigen (or epitope), includes but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well known in the art. For example, antigen-specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody).

“Co-stimulatory molecules” are involved in the interaction between receptor-ligand pairs expressed on the surface of antigen presenting cells and T cells. Research accumulated over the past several years has demonstrated convincingly that resting T cells require at least two signals for induction of cytokine gene expression and proliferation (Schwartz, R. H. (1990) Science 248: 1349-1356 and Jenkins, M. K. (1992) Immunol. Today 13:69-73). One signal can be produced by interaction of the TCR/CD3 complex with appropriate MHC/peptide complex. The second signal is not antigen specific and is termed the “co-stimulatory” signal. This signal was originally defined as an activity provided by bone-marrow-derived accessory cells such as macrophages and dendritic cells, the so called “professional” APCs. Several molecules have been shown to enhance co-stimulatory activity. These are heat stable antigen (HSA) (Liu, Y. et al. (1992) 3. Exp. Med. 175:437-445), chondroitin sulfate-modified MHC invariant chain (li-CS) (Naujokas, M. F. et al. (1993) Cell 74:257-268), intracellular adhesion molecule 1 (ICAM-1) (Van Seventer, G. A. (1990). Immunol. 144:4579-4586), B7-1, and B7-2/B70 (Schwartz, R. H. (1992) Cell 71:1065-1068). These molecules each appear to assist co-stimulation by interacting with their cognate ligands on the T cells. Co-stimulatory molecules mediate co-stimulatory signal(s), which are necessary, under normal physiological conditions, to achieve full activation of naïve T cells. One exemplary receptor-ligand pair is the B7 family of co-stimulatory molecule on the surface of APC5 and its counterreceptor CD28 or CTLA-4 on T cells (Freeman, et al. (1993) Science 262:909-911; Young, et al. (1992)]. Clin. Invest. 90:229 and Nabavi, et al. (1992) Nature 360:266-268). Other co-stimulatory molecules are CD40, and CD54. The term “costimulatory molecule” encompasses any single molecule or combination of molecules which, when acting together with a MHC/peptide complex bound by a TCR on the surface of a T cell, provides a co-stimulatory effect which achieves activation of the T cell that binds the peptide. The term thus encompasses B7, or other co-stimulatory molecule(s) on an antigen-presenting matrix such as an APC, fragments thereof (alone, complexed with another molecule(s), or as part of a fusion protein) which, together with MHC complex, binds to a cognate ligand and results in activation of the T cell when the TCR on the surface of the T cell specifically binds the peptide. It is intended, although not always explicitly stated, that molecules having similar biological activity as wild-type or purified co-stimulatory molecules (e.g., recombinantly produced or muteins thereof).

As used herein, the term “cytokine” refers to any one of the numerous factors that exert a variety of effects on cells, for example, inducing growth or proliferation. Non-limiting examples of cytokines which may be used alone or in combination in the practice of this invention include, interleukin-2 (IL-2), stem cell factor (SCF), interleukin-3 (IL-3), interleukin-6 (IL-6), interleukin-12 (IL-12), G-CSF, granulocyte macrophage-colony stimulating factor (GM-CSF), interleukin-1 alpha (IL-1a), interleukin-1L (IL-11), MIP-11, leukemia inhibitory factor (LIF), c-kit ligand, thrombopoietin (TPO) and flt3 ligand. One embodiment includes culture conditions in which an effective amount of IL-1β and/or IL-6 is excluded from the medium. Cytokines are commercially available from several vendors such as, for example, Genzyme (Framingham, Mass.), Genentech (South San Francisco, Calif.), Amgen (Thousand Oaks, Calif.), R&D Systems (Minneapolis, Minn.) and Immunex (Seattle, Wash.). It is intended, although not always explicitly stated, that molecules having similar biological activity as wild-type or purified cytokines (e.g., recombinantly produced or muteins thereof) are intended to be used within.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragment(s) thereof does not require “isolation” to distinguish it from its naturally occurring counterpart. In addition, a “concentrated”, “separated” or “diluted” polynucleotide, peptide, polypeptide, protein, antibody, or fragment(s) thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragment(s) thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as its glycosylation pattern. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature. A mammalian cell, such as dendritic cell is isolated if it is removed from the anatomical site from which it is found in an organism.

A “subject” is a vertebrate, preferably a mammal, more preferably a human. Mammals include, but are not limited to, murine, simians, humans, farm animals, sport animals, and pets.

The term “culturing” refers to the in vitro maintenance, differentiation, and/or propagation of cells or in suitable media. By “enriched” is meant a composition comprising cells present in a greater percentage of total cells than is found in the tissues where they are present in an organism.

A “composition” is intended to mean a combination of active agent and another compound or composition, inert (for example, a detectable agent or label) or active, such as an adjuvant. A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON'S PHARM. SCI., 18th Ed. (Mack Publ. Co., Easton (1990)).

RNA interference (RNAi) is a biological process in which RNA molecules inhibit gene expression, typically by causing the destruction of specific mRNA molecules or transcription and/or translation. The use of siRNA for gene manipulation of gene expression for manipulation or signaling of the stem cells to differentiate to osteo, neuro, vascular, pulmonary cell types.

An “effective amount” is an amount sufficient to effect beneficial or desired results, such as enhanced immune response, treatment, prevention or amelioration of a medical condition (disease, infection, etc.). An effective amount can be administered in one or more administrations, applications or dosages. Suitable dosages will vary depending on body weight, age, health, disease or condition to be treated and route of administration.

In one embodiment, “signaling” means contacting an immature or mature dendritic cell with an IFN-γ receptor agonist, a TNF-α receptor agonist, a CD40L polypeptide or other CD40 agonist or combination thereof. In one embodiment, such agonists are provided externally, (e.g., in the cell culture medium). In another embodiment, the polypeptide agonist is provided via transfection of an immature or mature dendritic cell with a nucleic acid encoding the polypeptide. Alternatively, a nucleic acid aptamer agonist could be provided in the medium or by transfection where the polypeptide(s) is provided by transfecting a dendritic cell with a nucleic acid encoding the polypeptide, signaling is effected upon translation of an mRNA encoding the polypeptide, rather than upon transfection with the nucleic acid. In one aspect, this provides a method for preparing enriched populations of mature dendritic cells (DCs) that induce potent immunostimulatory responses in vivo and/or in vitro.

Non-limiting examples of viral pathogens for use in the invention, for example, from which an antigenic polypeptide or polypeptides can be derived include, but are not limited to: adenoviruses; papillomaviruses; hepadnaviruses (e.g., hepatitis B); parvoviruses; pox viruses (e.g., small pox virus, vaccinia virus); Epstein-Barr virus; cytomegalovirus (CMV); herpes simplex viruses; roseolovirus; varicella zoster virus; filoviruses (e.g., Ebola virus and Marburg virus); paramyxoviruses (e.g., measles virus, mumps virus, Nipah virus, Hendra virus, human respiratory syncytial virus (RSV), parainfluenza viruses, Newcastle disease virus, and human metapneumovirus); orthomyxoviruses (e.g., influenza A, influenza B, and influenza C); rhabdoviruses (e.g., Lyssavirus, also known as rabies virus); arenaviruses (e.g., Lassa virus); coronaviruses (severe acute respiratory syndrome (SARS)); human enteroviruses; hepatitis A virus; human rhinoviruses; polio virus; retroviruses (e.g., human immunodeficiency virus 1 (HIV-1)); rotaviruses; flaviviruses, (e.g., West Nile virus, dengue virus, yellow fever virus); hepaciviruses (e.g., hepatitis C virus); and rubella virus.

Non-limiting examples of viral antigenic polypeptides include: influenza polypeptides such as hemagglutinin 1 (HA1), hemagglutinin 2 (HA2), and neuraminidase (NA); Lassa virus (LASV) polypeptides such as LASV glycoprotein 1 (gp1), LASV glycoprotein 2 (gp2), LASV nucleocapsid-associated protein (NP), LASV L protein, and LASV Z protein; SARS virus polypeptides such as SARS virus S protein; Ebola virus polypeptides such as Ebola virus GP2; measles virus polypeptides such as measles virus fusion 1 (F1) protein; HIV-1 polypeptides such as HIV Transmembrane™ protein, HIV glycoprotein 41 (gp41), HIV glycoprotein 120 (gp120); hepatitis C virus (HCV) polypeptides such as HCV envelope glycoprotein 1 (E1), HCV envelope glycoprotein 2 (E2), HCV nucleocapsid protein (p22); West Nile virus (WNV) polypeptides such as WNV envelope glycoprotein (E); Japanese encephalitis virus (JEV) polypeptides such as JEV envelope glycoprotein (E); yellow fever virus (YFV) polypeptides such as YFV envelope glycoprotein (E); tick-borne encephalitis virus (TBEV) polypeptides such as TBEV envelope glycoprotein (E); hepatitis G virus (HGV) polypeptides such as HGV envelope glycoprotein 1 (E1); respiratory synctival virus (RSV) polypeptides such as RSV fusion (F) protein; herpes simplex virus (HSV) polypeptides such as HSV-1 gD protein, HSV-1 gG protein, HSV-2 gD protein, and HSV-2 gG protein; hepatitis B virus (HBV) polypeptides such as HBV core protein; and Epstein-Barr virus (EBV) polypeptides such as EBV glycoprotein 125 (gp125).

Non-limiting examples of bacterial pathogens for use in the invention, for example, from which an antigenic polypeptide or polypeptides can be derived, include, but are not limited to, any pathogenic bacterial species from a genus selected from: Bacillus; Bordetella; Borrelia; Brucella; Burkholderia; Campylobacter, Chlamydia, Chlamydophila; Clostridium; Corynebacterium; Enterococcus; Escherichia; Francisella; Haemophilus; Helicobacter, Legionella; Leptospira; Listeria; Mycobacterium; Mycoplasma; Neisseria; Pseudomonas; Rickettsia; Salmonella; Shigella; Staphylococcus; Streptococcus; Treponema; Vibrio; and Yersinia.

Non-limiting examples of bacterial antigenic polypeptides include: outer membrane protein assembly factor BamA; translocation assembly module protein TamA; polypeptide-transport associated protein domain protein; bacterial surface antigen D15 from a wide variety of bacterial species; Bacillus anthracis polypeptides such as anthrax protective protein, anthrax lethal factor, and anthrax edema factor; Salmonella typhii polypeptides such as S1Da and S1Db; Vibrio cholerae polypeptides such as cholera toxin and cholera heat shock protein; Clostridium botulinum polypeptides such as antigen S and botulinum toxin; and Yersina pestis polypeptides such as F1, V antigen, YopH, YopM, YopD, and plasminogen activation factor (Pla).

A stem cell or dendritic delivery platform is similar to that of a cellular vaccine in that the antigen can be expressed through pathogen uptake, RNA uptake or DNA transfection and delivered by an ex vivo cultured cell.

The present invention utilizes stem cells and/or dendritic cells as a platform for a prophylactic vaccine or, in some cases a treatment (a therapeutic vaccine), for infectious disease. Cells that have been modified to express a foreign antigen are sufficient to elicit an antibody-mediated immune response without the need for additional adjuvants or boosting. As described herein, cells can be readily modified to secrete a foreign antigen (e.g., an immunogenic viral, or bacterial-derived polypeptide) and stimulate antigen-specific antibody production in vivo. The levels of antigen produced in this transient transfection are sufficient to induce an immunological response from a vaccine standpoint.

Immature DCs can be isolated or prepared from a suitable tissue source, including but not limited to, bone marrow, adipose and blood, containing DC precursor cells and differentiated in vitro to produce immature DC. For example, a suitable tissue source can be one or more of bone marrow cells, peripheral blood progenitor cells (PBPCs), peripheral blood stem cells (PBSCs), and cord blood cells. The tissue source can be a peripheral blood mononuclear cell (PBMC). The tissue source can be fresh or frozen. In another aspect, the cells or tissue source are pre-treated with an effective amount of a growth factor that promotes growth and differentiation of non-stem or progenitor cells, which are then more easily isolated from the cells of interest. These methods are known in the art and described briefly in Romani, et al. (1994) Exp. Med. 180:83 and Caux, C. et al. (1996) Exp. Med. 184:695. In one aspect, the immature DCs are isolated from peripheral blood mononuclear cells (PBMCs). In one embodiment, the PBMCs are treated with an effective amount of granulocyte macrophage colony stimulating factor (GM-CSF) in the presence or absence of interleukin 4 (IL-4) and/or IL-1β, so that the PBMCs differentiate into immature DCs. PBMCs are cultured in the presence of GM-CSF and IL-4 for about 4-7 days, about 5-6 days, to produce immature DCs. In some embodiments, the first signal is given at day 4, 5, 6, or 7, or at day 5 or 6. In addition, GM-CSF as well as IL-4 and/or IL-13 may be present in the medium at the time of the first and/or second signaling.

To increase the number of dendritic precursor cells in animals, including humans, one can pre-treat subjects with substances which stimulate hematopoiesis. Such substances include, but are not limited to G-CSF, and GM-CSF. The amount of hematopoietic factor to be administered may be determined by one skilled in the art by monitoring the cell differential of individuals to whom the factor is being administered. Typically, dosages of factors such as G-CSF and GM-CSF will be similar to the dosage used to treat individuals recovering from treatment with cytotoxic agents. As an example, GM-CSF or G-CSF can be administered for 4 to 7 days at standard doses prior to removal of source tissue to increase the proportion of dendritic cell precursors. U.S. Pat. No. 6,475,483 discloses that dosages of G-CSF of 300 micrograms daily for 5 to 13 days and dosages of GM-CSF of 400 micrograms daily for 4 to 19 days result in significant yields of dendritic cells.

This produces an enriched population of mature CD83⁺ CCR7⁺ dendritic cells that are potent immunostimulatory agents. This provides a method for preparing mature dendritic cells (DCs), comprising the sequential steps of: (a) signaling isolated immature dendritic cells (iDCs) with a first signal comprising an interferon gamma receptor (IFN-γR) agonist, and optionally a TNF-αR agonist, to produce IFN-7R agonist signaled dendritic cells; and (b) signaling said IFN-γR agonist signaled dendritic cells with a second transient signal comprising an effective amount of a CD40 agonist to produce CCR7+ mature dendritic cells. This further provides CD83+ CCR7− mature DCs and CD83+ CCR7+ mature DCs. In some embodiments, the CD83+ CCR7+ mature DCs and/or the CD83+ CCR7− mature DCs transiently express CD40L polypeptide. CD40L can be predominantly localized intracellularly, rather than on the cell surface. At least 60%, at least 70%, at least 80% or at least 90% of CD40L polypeptide is localized intracellularly.

In an alternative embodiment, the immature dendritic cells are signaled with an effective amount of a TNF-α receptor agonist followed by signaling with a CD40 agonist. This is a method for preparing mature dendritic cells (DCs), comprising sequentially signaling isolated immature dendritic cells with a first signal comprising a tumor necrosis factor alpha receptor (TNF-αR) agonist followed by a second signal comprising a CD40 agonist, wherein said signaling is in the absence of an effective amount of IL-1β and/or IL-6.

For either embodiment (IFN-γR agonist or TNF-ααR agonist as a first signal), the second CD40 agonist signal can be given to either CD83− CCR7− iDCs, or to CD83⁺ CCR7⁻ mature DCs. In one embodiment, the immature DCs and/or mature DCs are contacted with PGE2. In another embodiment the cells are contacted with PGE2 at about the same time that they receive the first signal (an IFN-γR agonist or TNF-αR agonist). In some embodiments, GM-CSF and at least one of IL-4 or IL-13 is present in the medium at the time the dendritic cells receive the first and second signals. In further embodiments, the method further comprises contacting the immature dendritic cells, signaled dendritic cells, and/or CCR7⁺ dendritic cells with a NKT cell ligand that can activate CD1d-restricted NKT cells and consequently potentiate innate and adoptive immunity. In some embodiments, the NKT cell ligand is a compound selected from the group consisting of: α-galactosylceramides, α-glucosylceramides, α-6-deoxygalactosylceramides, α-6-deoxygalactofuranosylceramides, β-6-deoxygalactofuranosylceramides, β-arabinosylceramides, α-C-galactosylceramides and α-S-galactosylceramides. A preferred compound is the α-galactosylceramide known as KRN7000 ((2S,3S,4R)-1-O-(alpha-D-galactopyranosyl)-2-(N-hexacosanoylamino)-1,3,4-octadecanetriol).

The compositions described herein are useful to raise an immune response in a subject by administering to the subject an effective amount of the enriched population of cells, e.g., DCs, modified DCs, or educated immune effector cells. The cells can be allogeneic or autologous. They can be administered to a subject to raise or induce an immune response in a subject comprising administering to the subject an effective amount of the enriched populations as described above. The cells can be allogeneic or autologous to the subject. They can also be used to educate immune effector cells such as T cells by culturing the immune effector cell in the presence and at the expense of a mature DC of this invention. The educated effector cells can also be used to enhance immunity in a subject by delivering to the subject an effective amount of these cells.

Methods of loading dendritic cells with antigens are known to those of skill in the art. In one embodiment, the dendritic cells are cultured in medium containing the antigen. The DCs then take up and process the antigen on the cell surface in association with MHC molecules. In one embodiment, DCs are loaded with antigen by transfection with a nucleic acid encoding the antigen. Methods of transfecting DCs are known to those of skill in the art.

T cells or dendritic cells can be administered directly to the subject to produce T cells active against a selected immunogen. Administration can be by methods known in the art to successfully deliver a cell into ultimate contact with a subject's blood or tissue cells.

The cells are administered in any suitable manner, often with pharmaceutically acceptable carriers. Suitable methods of administering cells in the context of the subject are available, and, although more than one route can be used to administer a particular cell composition, a particular route can often provide a more immediate and more effective reaction than another route. Routes of administration include, but are not limited to intradermal and intravenous administration.

Pharmaceutically acceptable carriers are determined in part by the particular composition being administered, as well as by the particular method used to administer the composition. Accordingly, there is a wide variety of suitable formulations of pharmaceutical compositions of the present invention. Most typically, quality controls (microbiology, clonogenic assays, viability tests), are performed and the cells are reinfused back to the subject, optionally preceded by the administration of diphenhydramine and hydrocortisone. See, for example, Korbling et al. (1986) Blood 67:529-532 and Haas et al. (1990) Exp. Hematol. 18:94-98.

Formulations suitable for parenteral administration, such as, for example, by intraarticular (in the joints), intravenous, intramuscular, intradermal, intraperitoneal, intranodal and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intraderaml and intravenous administration are the preferred method of administration for cells of the invention.

The dose of cells administered to a subject is in an effective amount, effective to achieve the desired beneficial therapeutic response in the subject over time, or to inhibit growth of cancer cells, or to inhibit infection.

For the purpose of illustration only, the method can be practiced by obtaining and saving blood samples from the subject prior to infusion for subsequent analysis and comparison. Generally at least about 10⁴ to 10⁶ and typically, between 1×10⁸ and 1×10¹⁰ cells are infused intravenously or intraperitoneally into a 70 kg patient over roughly 60-120 minutes. In one aspect, administration is by intravenous infusion. Vital signs and oxygen saturation by pulse oximetry are closely monitored. Blood samples are obtained 5 minutes and 1 hour following infusion and saved for analysis. Cell re-infusions are repeated roughly every month for a total of 10-12 treatments in a one year period. After the first treatment, infusions can be performed on an outpatient basis at the discretion of the clinician. If the re-infusion is given as an outpatient, the participant is monitored for at least 4 hours following the therapy.

For administration, cells can be administered at a rate determined by the effective dose, the LD-50 (or other measure of toxicity) of the cell type, and the side-effects of the cell type at various concentrations, as applied to the mass and overall health of the subject. Administration can be accomplished via single or divided doses. The cells can supplement other treatments for a condition by known conventional therapy, including cytotoxic agents, nucleotide analogues and biologic response modifiers. Similarly, biological response modifiers are optionally added for treatment by the cells of the invention. For example, the cells are optionally administered with an adjuvant, or cytokine such as GM-CSF, IL-12 or IL-2.

### EMBODIMENTS

In one embodiment, the therapy described herein educates immune effector cells. Mature dendritic cells can be generated from immature dendritic cells.

Cell therapy can utilize modified antigen presenting cells (APCs) or immune effector cells to initiate an immune response in a patient.

Dendritic cells (DC) are potent APCs involved in adaptive immunity. They coordinate the initiation of immune responses by naïve T cells and B cells and induce antigen-specific cytotoxic T lymphocyte (CTL) responses. DCs are specialized in several ways to prime helper and killer T cells in vivo. For example, immature DCs that reside in peripheral tissues are equipped to capture antigens and to produce immunogenic MHC-peptide complexes. In response to maturation-inducing stimuli such as inflammatory cytokines, immature DCs develop into potent T cell stimulators by upregulating adhesion and costimulatory molecules. At the same time, they migrate into secondary lymphoid organs to select and stimulate rare antigen-specific T cells. However, potent stimulation of T cells occurs after DC maturation, a process that increases the availability of MHC/peptide complexes on the cell surface, in addition to co-stimulatory molecules, that direct the effector function of the responding T-cells.

Co-stimulation is typical for a T cell to produce sufficient cytokine levels that induce clonal expansion. One characteristic of dendritic cells which makes them potent antigen presenting cells is that they are rich in co-stimulatory molecules of the immune response, such as the molecules CD80 and CD86, which activate the molecule CD28, on T lymphocytes. In return, T-helper cells express CD40L, which ligates CD40 on DCs. These mutual interactions between DCs and T-cells leads to ‘maturation’ of the former, and the development of effector function in the latter. The expression of adhesion molecules, like the molecule CD54 or the molecule CD11a/CD18, facilitate the co-operation between the dendritic cells and the T-cells. Another special characteristic of dendritic cells is to deploy different functions depending on their stage of differentiation. Thus, the capture of the antigen and its transformation are the two principal functions of the immature dendritic cell, whereas its capacities to present the antigen in order to stimulate the T cells increases as the dendritic cells migrate into the tissues and the lymphatic ganglia. This change of functionality corresponds to a maturation of the dendritic cell. Thus, the passage of the immature dendritic cell to the mature dendritic cell represents a step in the initiation of the immune response. Traditionally, this maturation was followed by monitoring the change of the surface markers on the DCs during this process. Some of the cell surface markers characteristic of the different stages of maturation of the dendritic cells are summarized in Table I, below. However, the surface markers can vary depending upon the maturation process.

TABLE I

| Cell type | Surface markers |
|---|---|
| Hematopoietic Stem | CD34⁺ |
| Cell | |
| Monocytes | CD14⁺⁺, DR⁺, CD86⁺, CD16^{+/-}, CD54⁺, CD40⁺ |
| Immature dendritic | CD14^{+/-}, CD16⁻, CD80^{+/-}, CD83⁻, CD86⁺, |
| cell | CD1a⁺, CD54⁺, DQ⁺, DR⁺⁺ |
| Mature dendritic | CD14⁻, CD83⁺⁺, CD86⁺⁺, CD80⁺⁺, |
| cell | DR⁺⁺⁺, DQ⁺⁺, CD40⁺⁺, CD54⁺⁺, |
| | CD1a^{+/-} |

Mature DCs may have benefits to immature DCs for immunotherapy. Only fully mature DC progeny lack GM-CSF Receptor (GM-CSF-R) and remain stably mature upon removal/in the absence of GM-CSF. Also, mature DCs have been shown to be superior in inducing T cell responses in vitro and in vivo. In contrast, immature DCs are reported to induce tolerance in vitro (Jonuleit et al. (2000) Exp. Med. 192:1213) as well as in vivo (Dhodapkar et al. (2001) Exp. Med. 193:233) by inducing regulatory T cells. Mature dendritic cells also are useful to take up and present antigen to T-lymphocytes in vitro or in vivo. The modified, antigen presenting DCs and/or T cells educated from these modified DCs have many applications, including diagnostic, therapy, vaccination, research, screening and gene delivery.

It is difficult to isolate mature dendritic cells from peripheral blood because less than 1% of the white blood cells belong to this category. Mature DCs are also difficult to extract from tissues. This difficulty, in combination with the potential therapeutic benefit of DCs in cell therapy, has driven research and development toward new methods to generate mature dendritic cells using alternative sources. Several methods are reported to produce mature DCs from immature dendritic cells.

For example, Jonuleit et al. (Eur J Immunol (1997) 12:3135-3142) disclose maturation of immature human DCs by culture in medium containing a cytokine cocktail (IL-1β, TNF-α, IL-6 and PGE2). WO 95/28479 discloses a process for preparing dendritic cells by isolating peripheral blood cells and enriching for CD34⁺ blood precursor cells, followed by expansion with a combination of hematopoietic growth factors and cytokines. European Patent Publication EP-A-0 922 758 discloses the production of mature dendritic cells from immature dendritic cells derived from pluripotential cells having the potential of expressing either macrophage or dendritic cell characteristics. The method requires contacting the immature dendritic cells with a dendritic cell maturation factor containing IFN-γ. European Patent Publication EP-B-0 633930 discloses the production of human dendritic cells by first culturing human CD34+ hematopoietic cells (i) with GM-CSF, (ii) with TNF-α and IL-3, or (iii) with GM-CSF and TNF-α to induce the formation of CD1a⁺ hematopoietic cells. Patent Publication No. 2004/0152191 discloses the maturation of dendritic cells by contacting them with RU 41740. U.S. Patent Publication No. 2004/0146492 discloses a process for producing recombinant dendritic cells by transforming hematopoietic stem cells followed by differentiation of the stem cells into dendritic cells by culture in medium containing GM-CSF. U.S. Patent Publication No. 2004/0038398 discloses methods for the preparation of substantially purified populations of DCs and monocytes from the peripheral blood of mammals. Myeloid cells are isolated from the mammal and DCs are separated from this population to yield an isolated subpopulation of monocytes. DCs are then enriched by negative selection with anti-CD2 antibodies to remove T cells.

Although mature DCs are functionally competent and are therefore useful to induce antigen-specific T cells, not all mature DCs are optimized to induce these responses. It has been shown that some mature DCS may also stimulate T helper cells by secreting IL-12. (Macatonia et al. (1995) Immunol. 154:507 1; Ahuja et al. (1998) Immunol. 161:868 and Unintford et al. (1999) Immunol. 97:588.) IL-12 also has been shown to enhance antigen-specific CD8+ T cell response to antigen in an animal model. (Schmidt et al. (1999) Immunol.; 163:2561.)

Mosca et al. (2000) Blood 96:3499, disclose that culture of DC in AIM V medium containing both soluble CD40L trimer and IFNγ 1b results in increased IL-12 expression in comparison to culture in medium containing only soluble CD40L trimer.

Koya et al. (2003) J. Immunother. 26(5):451 report that IL-12 expression can be enhanced by transducing immature DCs, in the presence of IFNγ, with a lentiviral vector encoding CD40 Ligand. Greater than 90% of the CD40L transduced DCs expressed CD83 on their cell surface. Unfortunately, lentiviral transduced cells are not suitable for therapeutic purposes, and proviral integration into the genome of the transduced cell can result in leukemia. Furthermore, persistent expression of CD40L may have detrimental effects on APC function and viability.

This work supplemented the earlier work of Mackey, et al. (1998) J. Immunol. 161:2094 who reported that in vivo, DCs require maturation via CD40 to generate anti-tumor immunity. Similarly, Kuniyoshi, J. S. et al. (1999) Cell Immunol. 193:48 have shown that DCs treated with soluble trimeric CD40 Ligand plus IFN-γ stimulated potent T-cell proliferation and induced T cells with augmented antigen-specific lysis. Kalady, M. F. et al. (2004) J. Surg. Res. 116:24, reported that human monocyte derived DCs transfected with mRNA encoding melanoma antigen MART-1 or influenza M1 matrix protein exposed to different maturation stimuli added either simultaneously or sequentially showed variability in antigen presentation, IL-12 secretion and immunogenicity of effector T cells raised in the presence of these DCs. This study showed that the application of a ‘cytokine cocktail’ consisting of IL-1β, TNF-α, IL-6 and PGE2, followed by extracellular soluble CD40L protein was superior to applying all the agents simultaneously. However, these authors did not study the combination of IFN-γ signaling with transient CD40L signaling in a sequential process. Moreover, despite the production of IL-12 when IFN-γ and CD40L are concomitantly added to the culture medium, the recent prior art shows that the resulting DCs are actually immunosuppressive, rather than pro-inflammatory (Hwu et al. (2000) J. Immunol. 164: 3596; Munn et al. (2002) 297:1867; and Grohmann et al. (2003) Trends Immunol. 24:242) due to the induction of an enzyme that metabolized tryptophan resulting in the starvation of responder T-cells that then fail to proliferate.

Potent immunostimulation occurs when immature dendritic cells are sequentially signaled with a first signal comprising an interferon gamma receptor (IFN-γR) agonist followed by a second signal comprising a CD40 agonist. Accordingly, this provides a method for preparing mature dendritic cells (DCs), comprising the sequential steps of: (a) signaling isolated immature dendritic cells (iDCs) with a first signal comprising an interferon gamma receptor (IFN-γR) agonist, and optionally a TNF-αR agonist, to produce signaled dendritic cells; and (b) signaling said signaled dendritic cells with a second transient signal comprising an effective amount of a CD40 agonist to produce CCR7⁺ mature dendritic cells.

In one embodiment, the immature DCs are further contacted with PGE2 and optionally with TNF-α. In alternative embodiments the method further comprises contacting the immature DCs, signaled DCs and/or CCR7⁺ mature dendritic cells with a compound selected from the group consisting of: galactosylceramides, glycosylceramides, galactofuranosylceramides, arabinopyranosylceramides, α-C-galactosylceramides and α-S-galactosylceramides. Preferably the compound is a galactosylceramide. Including, the galactosylceramide is (2S,3S,4R)-1-O-(alpha-D-galactopyranosyl)-2-(N-hexacosanoylamino)-1,3,4-octadecanetriol (KRN7000).

The IFN-γR agonist can be replaced by a tumor necrosis factor alpha receptor (TNF-αR) agonist. Thus, this provides a method for preparing an enriched population of mature dendritic cells (DCs), comprising sequentially signaling immature dendritic cells with a first signal comprising a tumor necrosis factor alpha receptor (TNF-αR) agonist followed by a second signal comprising a CD40 agonist, thereby preparing an enriched population of mature dendritic cells, wherein said signaling is in the absence of an effective amount of IL-1β or IL-6. In one embodiment, the immature DCs are further contacted with PGE2.

IFN-γR agonists include mammalian IFN-γ, such as human IFN-γ and active fragments thereof. TNF-αR agonists include but are not limited to mammalian TNF-α, such as human TNF-α and active fragments thereof. CD40 agonists include but are not limited to mammalian CD40 Ligands (CD40L), such as human CD40L and active fragments and variants thereof, as well as agonistic antibodies to CD40 receptor. Signaling can be initiated by providing the signaling agonist in the culture medium, introduction of the agonist into the cell, and/or upon translation within the dendritic cell of an mRNA encoding an agonistic polypeptide. The method can be practiced in vivo or ex vivo. Dendritic cells matured ex vivo according to the methods can then be administered to the subject to induce or enhance an immune response.

Each of the dendritic cells can be further modified by the administration of an immunogen to the DC. The DC will take up and process the immunogen, and display it on its cell surface. The immunogen can be delivered in vivo or ex vivo. The matured, cultured DCs can be administered to a subject to induce or enhance an immune response. In yet a further embodiment, the antigen loaded mature DCs are used to educate naïve immune effector cells.

Herein is described is a composition comprising in vitro matured dendritic cells, such as CD83⁺ CCR7⁻ mature DCs and CD83+ CCR7⁺ mature DCs. Mature dendritic cells express increased levels of IL-12 in comparison to immature dendritic cells, and/or express less than 500 pg IL-10 per million dendritic cells. The compositions are useful to raise an immune response in a subject by administering to the subject an effective amount of the population.

Active immunotherapy treatments are methods designed to activate the immune system to specifically recognize and destroy pathogen-infected cells. Harnessing the power of the immune system to treat chronic infectious diseases, including but not limited to Marburg virus, Ebola, Hantavirus, H5N1 strain of bird flu, Lassa virus, Junin virus, Dengue fever, Crimea-Congo fever virus, Bolivian hemorrhagic fever, Hep A, Hep B, Hep C, the rhino virus, polio, and chickenpox virus or Kyasanur Forest virus (KFD) virus is a novel treatment. In one embodiment, the immunotherapy treatment is coupled with the hyperthermia treatment described herein. The combination of the two treatments is further novel.

Active immunotherapy concepts are now being applied to develop therapeutic vaccines with the intention of treating and preventing chronic viral infection. Similar methods used in immunotherapy treatment will be effective not only for HIV, but also in deadly viruses such as Marburg virus, Ebola, Hantavirus, H5N1 strain of bird flu, Lassa virus, Junin virus, Dengue fever, Crimea-Congo fever virus, Bolivian hemorrhagic fever, Hep A, Hep B, Hep C, the rhino virus, polio, and chickenpox virus or Kyasanur Forest Virus (KFD) virus. The techniques disclosed herein can develop increased frequencies of immune cells in circulation that have the ability to specifically kill pathogen infected cells, such as HIV pathogen.

As an example, the current standard of treatment for HIV/AIDS is highly active antiretroviral therapy (HAART or ART), consisting of a combination of several antiretroviral drugs. Although this combination has been successful in reducing viral load (VL) in plasma and restoring some immune function, concerns regarding adverse effects associated with long-term usage of these antiretroviral drugs are growing. Specifically, a variety of metabolic disorders including HIV associated lipodystrophy, central adiposity, dyslipidaemia, hyperlipidaemia, hyperglycemia and insulin resistance have been reported as resulting from combination therapies. These reactions, combined with complex and cumbersome dosing regimen, can have an adverse impact on subject adherence to therapy. As a result, poor adherence has led to an increased rate of HIV and viral resistance, resulting in viral strains that have reduced sensitivity to the drugs and an increasing health care burden related to the treatment costs of the metabolic disorders.

Current chronic infectious disease treatment guidelines recommend starting anti-retrovirals for the treatment for HIV. There are currently five different “classes” of HIV drugs. Each class of drug attacks the virus at different points in its life cycle-so if one is taking HIV medication, you will generally take three different antiretroviral drugs from two different classes. This regimen is standard for HIV care, as currently no drug can cure HIV, and taking a single drug, by itself, won't stop HIV from harming its host. Taking three different HIV medications currently does the best job of controlling the amount of virus in the body and protecting the immune system. Taking more than one drug also protects one against HIV drug resistance. When HIV reproduces, it can make copies of itself that are imperfect- and these mutations may not respond to the drugs one takes to control their HIV. If one follows the three-drug regimen, the HIV in the body will be less likely to make new copies that don't respond to the HIV medications.

A combination of drugs including two nucleoside or nucleotide reverse transcriptase inhibitors (NRTIs) with either a protease inhibitor (PI) or a non-nucleoside reverse transcriptase inhibitor (NNRTI). Although these combinations are very effective in controlling HIV replication, resistance to NRTI can build up over time. Many NRTI and NNRTI resistance mutations confer cross-resistance to other drugs in that same class, thus limiting future viable antiretroviral options. Furthermore, NRTIs have poor tolerability and significant toxicities which can compromise compliance and promote drug resistance. In addition, mitochondrial dysfunction induced by NRTIs produces a spectrum of illnesses including peripheral neuropathy, myopathies, steatohepatitis, pancreatitis, lipoatrophy, tubular acidosis and lactacidosis. Because of issues relating to HAART or ART, new approaches are needed. One approach is to elicit cytotoxic T lymphocyte response through vaccines.

This can be applied to the treatment of other chronic viral infections such as Ebola and genetically-related viruses such as Marburg virus. The innate immune response is able to slow down viral replication and activate cytokines which trigger the synthesis of antiviral proteins. Cytokines are released by cells and affect the behavior of other cells, and sometimes the releasing cell itself.

Cytokines act through receptors, and play a role in the immune system; cytokines modulate the balance between cell-based immune responses, and they regulate the maturation, growth, and responsiveness of particular cell populations. Some cytokines enhance or inhibit the action of other cytokines in complex ways.

The adaptive immune system neutralizes virus particles and destroys infected cells. However, viruses have developed a number of countermeasures to avoid immune attack and stay moving targets for the immune system.

Because of the current shortcomings of antiviral therapy, a continuing and unmet medical need exists for new, improved, and alternative therapeutic treatment modalities for infection. Because of the negative aspects of NRT (an antiviral drug used against HIV; is incorporated into the DNA of the virus and stops the building process; results in incomplete DNA that cannot create a new virus; often used in combination with other drugs), a NRTI-sparing regimen that is able to control viral replication would be a very useful addition to the HIV treatment armamentarium. Such additions could include treatment vaccines that are designed to induce potent cytotoxic T-cell responses. The present invention addresses these and other unmet needs by providing a novel therapeutic immunogenic composition and/or vaccine optionally co-administered with an antiviral backbone monotherapy regimen optionally coupled with perfusion hyperthermia for treatment of infection, such as the treatment of chronic infectious diseases, including but not limited to Marburg virus, Ebola, Hantavirus, H5N1 strain of bird flu, Lassa virus, Junin virus, Dengue fever, Crimea-Congo fever virus, Bolivian hemorrhagic fever, HIV, or Hep A, Hep B, Hep C, the rhino virus, polio, and chickenpox virus Kyasanur Forest Virus (KFD).

The Ebola viruses, and the genetically related Marburg virus, viruses of the Filoviridae family, are associated with outbreaks of highly lethal hemorrhagic fever in humans and primates in North America, Europe, and Africa (Peters, C. J. et al. in: Fields Virology, eds. Fields, B. N. et al. 1161-1176, Philadelphia, Lippincott-Raven, 1996; Peters, C. J. et al. 1994 Semin Virol 5: 147-154). Ebola viruses are negative-stranded RNA viruses comprised of five subtypes, including those described in the Zaire, Sudan, Reston, Ivory Coast and Bundibugyo episodes (Sanchez, A. et al. 1996 PNAS USA 93:3602-3607). The Ebola virus, was first recognized during an outbreak in 1976 in the Ebola River valley of Zaire (currently the Democratic Republic of the Congo), Africa. Mortality rates vary between different species, spanning from approximately 35 to 90% for the most virulent ones, Zaire and Sudan. The development of effective vaccines and/or drugs is a high priority. The Ebola (EBOV) and Marburg (MARV) viruses have also been categorized as priority class A pathogens due to their virulence, ease of dissemination, lack of effective countermeasures to prevent or treat them, and their potential to cause public panic and social disruption. Although several subtypes have been defined, the genetic organization of Ebola viruses is similar, each containing seven linearly arrayed genes. Among the viral proteins, the envelope glycoprotein exists in two alternative forms, a 50-70 kilodalton (kDa) secreted protein of unknown function encoded by the viral genome and a 130 kDa transmembrane glycoprotein generated by RNA editing that mediates viral entry.

Replication-defective adenovirus vectors (rAd) are powerful inducers of cellular immune responses and have therefore come to serve as useful vectors for gene-based vaccines, particularly for lentiviruses and filoviruses, as well as other nonviral pathogens (Shiver, et al, (2002) Nature 415(6869): 331-5; (Hill, et al, Hum Vaccin 6(1): 78-83; Sullivan, et al, (2000) Nature 408(6812): 605-9; Sullivan et al, (2003) Nature 424(6949): 681-4; Sullivan, et al, (2006) PLoS Med 3 (6): e177; Radosevic, et al, (2007); Santra, et al, (2009) Vaccine 27(42):5837-45. Adenovirus-based vaccines have several advantages as human vaccines since they can be produced to high titers under GMP conditions and have proven to be safe and immunogenic in humans (Asmuth, et al, J Infect Dis 201 (1): 132-41; Kibuuka, et al, J Infect Dis 201 (4): 600-7; Koup, et al., PLoS One 5 (2): e9015; Catanzaro, et al, (2006) J Infect Dis 194(12): 1638-49; Harro, et al, (2009) Clin Vaccine Immunol 16(9): 1285-92). While most of the initial vaccine work was conducted using rAd5 due to its significant potency in eliciting broad antibody and CD8⁺ T cell responses, pre-existing immunity to rAd5 in humans may limit efficacy (Catanzaro, (2006); Cheng, et al, (2007) PLoS Pathog 3 (2): e25; McCoy, et al, (2007) J Virol 81 (12): 6594-604; Buchbinder, et al, (2008) Lancet 372(9653): 1 881-93). This property might restrict the use of rAd5 in clinical applications for many vaccines that are currently in development including Ebola virus (EBOV) and Marburg virus (MARV).

To circumvent the issue of pre-existing immunity to rAd5, several alternative vectors are currently under investigation. These include adenoviral vectors derived from rare human serotypes and vectors derived from other animals such as chimpanzees (Vogels, et al, (2003) J Virol 77(15): 8263-71; Abbink, et al, (2007) J Virol 81: 4654-63; Santra, (2009) Vaccine 27(42): 5837-45). Chimpanzee adenoviral vectors are also described in WO 2010/086189, WO 2005/071093 and WO 98/10087.

The RNA may encode structural gene products of any bacterial or viral species such as filovirus species. There are five species of Ebola viruses, Zaire (type species, also referred to herein as ZEBOV), Sudan (also referred to herein as SEBOV), Reston, Bundibugyo, and Ivory Coast. There is a single species of Marburg virus (also referred to herein as MARV). The particular antigen expressed in the vectors are not a critical aspect. The adenoviral vectors can be used to express proteins comprising an antigenic determinant of a wide variety of filovirus antigens. The vectors include nucleic acid encoding the transmembrane form of the viral glycoprotein (GP). The vectors can encode the secreted form of the viral glycoprotein (SGP), or the viral nucleoprotein (NP).

The RNA molecules may be mutated, as long as the modified expressed protein elicits an immune response against a pathogen or disease. The protein may be mutated so that it is less toxic to cells (see e.g., WO2006/037038) nucleic acid molecules encoding GP, SGP and NP of the Zaire, Sudan and Ivory Coast Ebola strains may be combined in any combination, in one vaccine composition. Exposure to certain adenoviruses can result in immune responses against certain adenoviral serotypes, which can affect efficacy of adenoviral vaccines.

Therapeutic immunization (vaccine) and administration: The method is produced using immature dendritic cells which develop to mature dendritic cells when contacted with a maturation composition (such as viral RNA). These cells are administered through injection to a vein such as median cubital or radial artery, ulnar artery in the arm or any readily accessible vein or artery in the event of a malnourished or dehydrated patient.

Immature dendritic cells can be obtained from a population of dendritic cell precursors. In one embodiment, dendritic cell precursor is a cell that can differentiate into an immature dendritic cell in four weeks or less, for example, in 20 days or less, including, in 18 days or less, such as, in 16 days or less. In one embodiment, the dendritic cell precursor differentiates into an immature dendritic cell in the presence of GM-CSF and IL-4 in less than seven days, such as, in five days.

In one embodiment, the population of dendritic precursor cells is a population of monocytic dendritic cell precursors. In one embodiment, the monocytic dendritic cell precursors are derived from peripheral blood mononuclear cells (PBMCs). The PBMCs can be obtained either from whole blood diluted 1:1 with buffered saline or from leukocyte concentrates (“buffy coat” fractions, MSKCC Blood Bank) by standard centrifugation over Ficoll-Paque PLUS (endotoxin-free, catalogue number 17-1440-03, Amersham Pharmacia Biotech AB, Uppsala, SE). MoDC precursors are tissue culture plastic-adherent (catalogue number. 35-3003, Falcon, Becton-Dickinson Labware Inc., Franklin Lakes, N.J., US) PBMCs, and can be cultured in complete RPMI 1640 plus 1% normal human serum (NHS) (or 10% fetal bovine serum) in the presence of GM-CSF (1000 IU/mL) and IL-4 (500 IU/mL) with replacement every 2 days as described. See Thumer B, et al., J. Immunol. Meth. 1999; 223: 1-15 and Ratzinger G, et al, J. Immunol. 2004; 173:2780-2791.

Purified monocyte populations can be isolated from PBMCs with CD14⁺ antibodies prior to the culture to obtain immature dendritic cells. Monocytes are usually identified in stained smears by their large bilobate nucleus. In addition to the expression of CD14, monocytes express also, among others, one or more of the following surface markers: 125I-WVH-1, 63D3, adipophilin, CB12, CD11a, CD11b, CD15, CD54, Cd163, cytidine deaminase, and FIt-I. See Feyle D, et al., Eur. J. Biochem. 1985; 147:409-419, Malavasi F, et al., Cell Immunol. 1986; 97(2):276-285, Rupert J, et al., Immunobiol. 1991; 182(5):449-464; Ziegler-Heitbrock H, J. Leukoc. Biol. 2000; 67:603-606, and Pilling D, et al, PLoS One 2009; 4 (10):e-7475. In general, monocytic dendritic cell precursors may be identified by the expression of markers such as CD13 and CD33. Myeloid dendritic precursors may differentiate into dendritic cells via CD14 or CD1a pathways. Accordingly, a dendritic precursor cell of the cell may be a CD14⁺CD1a⁻ dendritic precursor cell or a CD14⁻CD1a⁺ dendritic precursor cell. In certain embodiments of the invention, a myeloid dendritic precursor cell may be characterized by the expression of SCA-1, c-kit, CD34, CD 16, and CD14 markers. In one embodiment, the myeloid dendritic precursor cell is a CD14⁺ monocyte. The CD14⁺ monocyte may also express the GM-CSF receptor.

Herein, CD8⁺ T cells are being used for therapy as they are intrinsically different in their proliferative responses, when comparing the proliferation and differentiation of Ag-specific CD4 and CD8 T cells following infection. Results show that CD4 T cells responding to infection divide a limited number of times, with progeny exhibiting proliferative arrest in early divisions. Even with increased infectious doses, CD4 T cells display this restricted proliferative pattern and are not driven to undergo extensive clonal expansion. This is in contrast to CD8 T cells, which undergo extensive proliferation in response to infection. These differences are also evident when CD4 and CD8 T cells receive uniform anti-CD3 (which activates resting T cell lymphocytes by CD3 antibodies) stimulation in vitro. Together, these results suggest that CD4 and CD8 T cells are programmed to undergo limited and extensive proliferation, respectively, to suit their function as regulator and effector cells.

A cocktail of various stem cells loaded the cocktail including roughly 30-40% mesenchymal (allows for targeting of lineages within various systems—primarily cardio, blood vessel, and neuronal), about 50-60% hematopoietic for all conditions lying within blood-HIV/Ebola) and be used. The percentages can be altered based on the specific nature of target pathogen, and the other 10% osteo (with a focus on bone marrow differentiation lineage 10% because the penetration in the osteo niche is substantially more difficult (treating a condition in the marrow rather than blood would heavily favor the use of the stem cell). The reason for this combination is so that the cell can be targeted to different tissues creating self-assembly and allowing for error correction and the cells to go from one state to another targeting program (HIV, Ebola etc.). HIV and Ebola cell transduction vectors are particularly desirable because of their ability to be pseudotyped to infect non-dividing hematopoietic stem cells (CD34⁺). This is done by transducing the packaging cell line used to package the vector with a nucleic acid which encodes the vesicular stomatitis virus (VSV) envelope glycoprotein, which is then expressed on the surface of the HIV vector. VSV infects CD34⁺ cells, and pseudotype HIV-2 vectors expressing VSV envelope proteins are competent to transduce these cells. CD34⁺ cells can be used as target cells for ex vivo gene therapy, because these cells differentiate into many different cell types, and because the cells are capable of re-engraftment into a patient undergoing ex vivo therapy. Stem cells differentiate in vivo into a variety of immune cells, including CD8⁺ cells which are the primary targets for HIV and Ebola infection. Further these cells can be extracted from blood as opposed to bone marrow, extracting from blood is far less invasive and much safer for both the patient and those doing the procedure.

One embodiment is directed to using, for example, hematopoietic stem cells in combination with inert 3D and 4D printed mold of a virus to promote differentiation of the stem cells pre-exposed to the virus. The 3D/4D printed mold of pathogen should be that of the most common form or if extremely mutagenic then the most primary state as that form will most likely cause the CD8⁺/CD4⁺ cells to express interest in markers not yet removed from the mutagenic lineage of the virus.

Human stem cells can be genetically engineered into ‘warrior’ cells that fight deadly viruses such as HIV, Ebola, Marburg ETC- and the new cells/warriors can attack HIV, Ebola, Marburg ETC-infected cells inside a living creature. Much HIV research focuses on vaccines or drugs that slow the virus's progress—but this new technique offers a cure. These results demonstrate use of this technology to engineer the human immune response to combat viral infections and suggest that genetic engineering via HSCs and other stem cells may allow tailoring of the immune response to target and eradicate, for example, HIV, Ebola, Marburg etc.

The immature dendritic cells used in the beginning can be autologous to the subject to be treated. In other embodiments, the immature dendritic cells used as starting material are heterologous dendritic cells. For example, if graft-versus-host disease is to be treated, the immature dendritic cells that are being used as starting material are dendritic cells that were obtained from the donor. The subject can be, for instance, a mouse, a rat, a dog, a chicken, a horse, a goat, a donkey, or a primate (e.g., human). In one embodiment, the subject is a human. In another embodiment, the immature dendritic cell is a monocyte-derived immature dendritic cell.

The first step is contacting said immature dendritic cells with an immunogen comprising said antigen under conditions adequate for maturation of said antigen presenting cell and under conditions which prevent the adhesion of the cells to the substrate. As a result, an antigen-loaded antigen-presenting cell is obtained.

At the end of the incubation time a mature antigen-loaded dendritic cell is obtained (i.e. a mature dendritic cell carrying the antigen of interest). Maturation of dendritic cells can be monitored by methods known in the art. mDCs surface markers can be detected in assays such as flow cytometry and immunohistochemical staining. The mDCs can also be monitored by cytokine production (e.g. by ELISA, another immune assay, or by use of an oligonucleotide array). The maturation of a dendritic cell can be further confirmed by immunophenotyping. An immature dendritic cell may be distinguished from a mature dendritic cell, for example, based on markers selected from the group consisting of CD80 and CD86. An immature dendritic cell is weakly positive and preferably negative for these markers, while a mature dendritic cell is positive.

In a culture having a population of immature dendritic cells, conditions adequate for maturation are such where the maturation of at least about 50%, at least about 60%>, at least about 70%>, at least about 80%>, at least about 90%>, or about 100% of immature dendritic cells, is achieved.

First, recovering the immunogen-pulsed dendritic cells, said recovery can be carried out by any method known in the art. In one embodiment, the recovery of the immunogen-pulsed dendritic cells is carried out by immunoisolation using antibodies specific for markers of mature dendritic cells such as one or more of the group consisting of CD4, CD8, CD54, CD56, CD66b, and CD86.

In one embodiment, the immunogen to be loaded into a dendritic cell is a viral particle, such as a retroviral viral particle. In another embodiment, the immunogen is a lentivirus particle, such as an HIV viral particle. In one embodiment, the immunogen is an HIV-1 viral particle. HIV-1 virus binds with and subsequently infects human CD4 cells through the use of a co-receptor on the cell surface. Different strains of HIV-1 use different co-receptors to enter human CD4 cells. Thus, HIV-1 virus can be CCR5-tropic when the virus strain only uses the C—C chemokine receptor type 5 (CCR5) co-receptor to infect CD4 cells; CXCR4-tropic when a virus strain only uses the C—X—C chemokine receptor type 4 (CXCR4) co-receptor to infect the CD4 cells; and dual-tropic when the virus strain can use either the CCR5 or CXCR4 co-receptor to infect CD4 cells. See Whitcomb J, et al, Antimicrob. Agents Chemother. 2007; 51(2):566-575. There are available several assays to distinguish between different tropic viruses (e.g. Trofile®, Monogram Biosciences, Inc., San Francisco, Calif., US). In one embodiment, the HIV-1 virus is selected from a CXCR4-tropic virus and a CCR5-tropic virus; in one embodiment it is a CXCR4-tropic virus.

In another embodiment, the immunogen is an inactivated viral particle or lysate, such as an HIV particle or a lysate of essentially inactivated HIV. The virus or the lysate thereof can be inactivated using conventional means, such as heat, chemical agents and photochemical agents.

An inactivated virus is not detectably infectious in vitro. To quantify the reduction in the infective dose produced by the inactivation process applied and to quantify the residual infective dose that remains in the sample after the inactivation, the inactivated virus, e.g., HIV, is submitted to an assay. Methods that can be used to this purpose are known in the art. See Agrawal K, et al, PLoS One. 2011; 6 (6):e21339. The methods include the use of inactivated supernatants for infecting permissible cells followed by detection of the newly formed virus. Said detection can be carried out by measuring the number of, for example, HIV RNA copies/mL produced by the cells or the amount of, for example, HIV p24 antigen/mL of supernatant by the ELISA method. The detection of the production of, for example, HIV p24 antigen can be carried out, for instance, by ELISA as described in the experimental part of the present invention.

The inactivation step is carried out for sufficient time so as to result in an decrease in infectivity of the supernatant with respect to a control supernatant (i.e. a supernatant which has not been treated with the inactivating agent or which has been treated under similar conditions with the vehicle in which the inactivating agent is provided) of at least about 0%, at least about 20%, at least 3 about 0%, at least about 40%, at least about 50%, at least 6 about 0%, at least about 70%, at least about 80%, at least about 90% or at least about 100%. Suitable methods for assessing, for example, HIV inactivation, entails, without limitation, taking blood cultures followed by culturing in a T cell media and measuring infectivity. An alternative method is to determine the virus copies that are present in the blood before and after the inactivation attempt or treatment in a periodic fashion (e.g. every 1-7 days).

In one embodiment, the immunogen is a heat-inactivated virus or virus lysate. Viruses, such as HIV-1, may be heat-inactivated by several known protocols in the art. See Harper J, et al., J. Virol. 1978; 26(3):646-659, Einarsson R, et al., Transfusion 1989; 29(2): 148-152, and Gil C, et al, Vaccine 2011; 29(34): 5711-5724.

The immunogen can be a chemically-inactivated virus or virus lysate. The inactivation may be attained by incubating the virus with a chemical agent. In a further aspect the mixture of the virus and the chemical agent is optionally irradiated. For example, the mixture is irradiated with ultraviolet light until the virus is inactivated.

The chemical agent can be a zinc finger-modifying compound. The term “zinc finger-modifying compound” refers to a compound that covalently modifies the zinc fingers in the nucleocapsid protein of, for example, HIV virions, thereby inactivating infectivity. The advantage of such a mode of inactivation is that the conformational and functional integrity of proteins on the virion surface is preserved. A number of compounds have been identified that act via a variety of different mechanisms to covalently modify the nucleocapsid zinc fingers, resulting in ejection of the coordinated zinc and loss of infectivity. Despite differences between detailed mechanisms of action for these compounds, the common mechanistic feature involves a preferential chemical attack on the zinc-coordinating cysteine sulfurs in the residues that make up the nucleocapsid protein zinc fingers. See Rossio J, et al, J. Virol. 1998; 72(10):7992-8001).

Suitable zinc finger-modifying compounds for use in the process include, without limitation: (i) a C-nitroso compound, (ii) azodicarbonamide, (iii) a disulphide having the structure R—S—S—R, (iv) a maleimide having the structure:

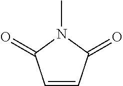

(v) an alpha-halogenated ketone having the structure

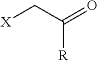

(vi) an hidrazide having the formula R—NH—NH—R, (vii) nitric oxide and derivatives thereof containing the NO group, (viii) cupric ions and complexes containing Cu²⁺, (ix) ferric ions and complexes containing Fe³⁺, wherein R is any atom or molecule and X is selected from the group consisting of F, I, Br and Cl.

Examples of disulfide compounds include, but are not limited to, the following: tetramethylthiuram disulfide, tetraethylthiuram disulfide, tetraisopropylthiuram disulfide, tetrabutylthiuram disulfide, dicyclopentamethylenethiuram disulfide, isopropylxanthic disulfide, O,O-diethyl dithiobis-(thioformate), benzoyl disulfide, benzoylmethyl disulfide, formamidine disulfide 2HC1, 2-(diethylamino)ethyl disulfide, aldrithiol-2, aldrithiol-4, 2,2-dithiobis(pyridine N-oxide), 6,6-dithiodinicotinic acid, 4-methyl-2-quinolyl disulfide, 2-quinolyl disulfide, 2,2-dithiobis(benzothiazole), 2,2-dithiobis(4-tert-butyl-1-Isopropyl)-imidazole, 4-(dimethylamino)phenyl disulfide, 2-acetamidophenyl disulfide, 2,3-dimethoxyphenyl disulfide, 4-acetamidophenyl disulfide, 2-(ethoxycarboxamido)phenyl disulfide, 3-nitrophenyl disulfide, 4-nitrophenyl disulfide, 2-aminophenyl disulfide, 2,2-dithiobis(benzonitrile), /-tolyl disulfoxide, 2,4,5-trichlorophenyl disulfide, 4-methylsulfonyl-2-nitrophenyl disulfide, 4-methylsulfonyl-2-nitrophenyl disulfide, 3,3-dithiodipropionic acid, N,N-diformyl-L-cystine, trans-1,2-dithiane-4,5-diol, 2-chloro-5-nitrophenyl disulfide, 2-amino-4-chlorophenyl disulfide, 5,5-dithiobis(2-nitrobenzoic acid), 2,2-dithiobis(1-naphtylamine), 2,4-dinitrophenyl-/-tolyl disulfide, 4-nitrophenyl-/-tolyl disulfide, and 4-chloro-3-nitrophenyl disulfideformamidine disulfide dihydrochloride.

In one embodiment, the disulfide compound is selected from the group of disulfiram or aldrithiol-2 (2,2′-dithiodipyridine). In another embodiment, the zinc finger-modifying compound is aldrithiol-2. In one embodiment, the zinc finger-modifying compound is disulfiram.

An example of a maleimide is N-ethylmaleimide.

An example of a hydrazide is 2-(carbamoylthio)-acetic acid 2-phenylhydrazide.

In another embodiment, the inactivation is photochemical. In a one embodiment, the photochemical inactivation is carried out by using a psoralen compound and irradiating the mixture of the virus and the psoralen compound at a wavelength capable of activating said psoralen compound. Psoralens which may be used in the inactivation step include psoralen and substituted psoralens, in which the substituent may be alkyl, particularly having from one to three carbon atoms (e.g. methyl); alkoxy, particularly having from one to three carbon atoms (e.g. methoxy); and substituted alkyl having from one to six, more usually from one to three carbon atoms and from one to two heteroatoms, which may be oxy, particularly hydroxy or alkoxy having from one to three carbon atoms (e.g. hydroxy methyl and methoxy methyl), or amino, including mono- and dialkyl amino or aminoalkyl, having a total of from zero to six carbon atoms (e.g. aminomethyl). There will be from 1 to 5, usually from 2 to 4 substituents, which will normally be at the 4, 5, 8, 4′ and 5′ positions, particularly at the 4′ position. Examples of psoralens include psoralen; 5-methoxypsoralen; 8-methoxy-psoralen; 5,8-dimethoxypsoralen; 3-carbethoxypsoralen; 3-carbethoxy-pseudopsoralen; 8-hydroxypsoralen; pseudopsoralen; 4,5′,8-trimethyl-psoralen; allopsoralen; 3-aceto-allopsoralen; 4,7-dimethyl-allopsoralen; 4,7,4′-trimethyl-allopsoralen; 4,7,5′-trimethyl-allopsoralen; isopseudopsoralen; 3-acetoisopseudopsoralen; 4,5′-dimethyl-isopseudo-psoralen; 5′,7-dimethyl-isopseudopsoralen; pseudoisopsoralen; 3-aceto-seudoisopsoralen; 3/4′,5′-trimethyl-aza-psoralen; 4,4′,8-trimethyl-5′-amino-methylpsoralen; 4,4′,8-trimethyl-phthalamyl-psoralen; 4,5′,8-trimethyl-4′-aminomethyl psoralen; 4,5′,8-trimethyl-bromopsoralen; 5-nitro-8-methoxy-psoralen; 5′-acetyl-4,8-dimethyl-psoralen; 5′-aceto-8-methyl-psoralen; and 5′-aceto-4,8-dimethyl-psoralen. In a more preferred embodiment the psoralen compound is amotosalen, preferably in salt form as amotosalen hydrochloride (S-59). No in vivo pharmacological effect of residual amotosalen is intended.

The time of UV irradiation will vary depending upon the light intensity, the concentration of the psoralen, the concentration of the virus, and the manner of irradiation of the virus receives, where the intensity of the irradiation may vary in the medium. The time of irradiation will be inversely proportional to the light intensity. The total time will usually be at least about 5 minutes and no more than about 30 minutes, generally ranging from about 5 to 10 minutes.

The light, which is employed, will generally have a wavelength in the range from about 300 nm to 400 nm. Usually, an ultraviolet light source will be employed together with a filter for removing UVB light. The intensity will generally range from about 150 μW/cm² to about 1500 μW/cm² although in some cases, it may be higher.

It may be desirable to remove the unexpended psoralen or its by-products from the irradiation mixture. This can be readily accomplished by one of several standard laboratory procedures such as dialysis across an appropriately sized membrane or through an appropriately sized hollow fiber system after completion of the irradiation. Alternatively, affinity methods can be used for removing one or more of the low molecular weight materials.

Antigen-loaded dendritic cells and dendritic cell vaccines/immunogenic compositions: The methods described herein allow for obtaining antigen-pulsed dendritic cells. Thus, in another aspect, this relates to an antigen-pulsed dendritic cell which can be obtained by using the methods described herein.

Dendritic cells suitable can be of different types such as, without limitation, myeloid DCs, plasmacytoid DCs, Langerhans cells and insterstitial DCs. The most potent of the professional APCs are DCs of myeloid origin. Thus, in one embodiment, DCs are myeloid DCs. Dendritic cells can be identified by their particular profile of cell surface markers. This determination can be carried out, for example, by means of flow cytometry using conventional methods and apparatuses. For example, a fluorescent-activated cell sorting (Becton Dickinson Calibur FACS, Becton-Dickinson Labware Inc., Franklin Lakes, N.J., US) system with commercially available antibodies following protocols well established in the art can be used. Thus, the cells presenting a signal for a specific cell surface marker in the flow cytometry above the background signal can be selected. The background signal is defined as the signal intensity given by a nonspecific antibody of the same isotype as the specific antibody used to detect each surface marker in the conventional FACS analysis. In order for a marker to be considered positive, the observed specific signal has to be more than about 20%, about 30%, 40%, 50%, 60%, 70%, 80%, 90%, 500%, 1000%, 5000%, 10000% or above, intense in relation to the intensity of the background signal using conventional methods and apparatuses.

Dendritic cells have profound abilities to induce and coordinate T cell immunity: This makes them ideal biological agents for use in immunotherapeutic strategies to augment T cell immunity to HIV infection, as well as other blood borne pathogens and diseases such as Ebola and Marburg virus. Thus, described herein is relates to an immunogenic composition and/or vaccine comprising the antigen-pulsed dendritic cells which can be obtained using the methods described herein.

Said dendritic cell vaccine or immunogenic composition can be autologous to the subject. As used herein, the term “autologous” is meant to refer to any material derived from the same subject to which it is later to be reintroduced into the subject.

In another aspect, the dendritic cell vaccine or immunogenic composition where the immunogen is an viral immunogen, such as an HIV immunogen, for use in the treatment or prevention of an HIV-infection or a disease associated with an HIV infection or Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Hep A, Hep B, Hep C, the rhino virus, polio, and chickenpox virus or Malaria.

In another aspect, the use of a dendritic cell vaccine or immunogenic composition where the immunogen is an HIV immunogen for the preparation of a medicament for the treatment in a subject of an HIV-1 infection or a disease associated with an HIV infection or Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Hep A, Hep B, Hep C, the rhino virus, polio, and chickenpox virus or Malaria.

Also in the instance of a subject afflicted with an HIV-1 infection or a disease associated with an HIV infection comprising the administration to said subject of a dendritic cell vaccine or immunogenic composition where the immunogen is HIV or Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Hep A, Hep B, Hep C, the rhino virus, polio, and chickenpox virus or Malaria.

The dendritic cell vaccine or immunogenic composition can be a therapeutic vaccine, that is, a material given to already HIV infected subjects as well as Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Malaria that have developed AIDS or Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Hep A, Hep B, Hep C, the rhino virus, polio, and chickenpox virus or Malaria.

The dendritic cell vaccine or immunogenic composition can be a prophylactic AIDS vaccine designed to be administered to an already HIV infected subject that has not developed AIDS or has contracted Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Hep A, Hep B, Hep C, the rhino virus, polio, and chickenpox virus or Malaria.

In one embodiment, the dendritic cell vaccine or immunogenic composition is administered to a subject that is under antiretroviral therapy (ART), and preferably, under Highly Active Antiretroviral Therapy (HAART). In another embodiment the dendritic cell vaccine is administered to a subject that has discontinued antiretroviral therapy. In one embodiment dendritic cell vaccine or immunogenic composition is administered to a subject that is undergoing treatment for Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Hep A, Hep B, Hep C, the rhino virus, polio, and chickenpox virus or Malaria.

Accordingly, the therapeutic vaccine finds application to reduce the replication of virus, such as HIV-1, in already infected subjects and limit the infectivity of virus in a vaccinated subject.

The dendritic cell vaccine or immunogenic composition can comprise an antigen-loaded dendritic cell preparation comprising an immunogenically effective amount of an essentially inactivated virus, such as HIV or Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Hep A, Hep B, Hep C, the rhino virus, polio, and chickenpox virus or Malaria and a pharmaceutically acceptable carrier.

In another embodiment, the dendritic cell-based vaccines or immunogenic composition can be administered by, for example, direct delivery of the APC loaded with inactivated subtype-specific HIV (e.g. by a subcutaneous injector) to a subject by methods known in the art.

An individual can be treated with APCs loaded with inactivated HIV of a specific subtype. The APCs are first loaded with the inactivated HIV ex vivo. The loaded APCs are then administered to the subject by any suitable technique. In one embodiment, the loaded APCs are injected subcutaneously, intradermally or intramuscularly into the individual, for example, by a subcutaneous injection. In one embodiment, the APCs are obtained by sampling PBMCs previously from the subject under treatment. The monocytes (CD14⁺) isolated from the PBMCs are differentiated to immature dendritic cells which are then developed into mature dendritic cells.

In another embodiment, composition includes an adjuvant to induce a cellular immune response against virus/bacteria, such as HIV-1 or any other pathogen. Suitable adjuvants include complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, conventional bacterial products (e.g. cholera toxin, heat-labile enterotoxin, attenuated or killed BCG (Bacillus Calmette-Guerin) and Cory b acterium parvum, or BCG derived proteins), biochemical molecules (e.g. TNF-α, IL-1-, IL-6, PGE2, or CD40L), or oligodeoxynucleotides containing a CpG motif. Examples of materials suitable for use in vaccine compositions have been disclosed previously. See Osol A, Ed., Remington's Pharmaceutical Sciences (Mack Publishing Co., Easton, Pa., US, 1980, pp. 1324-1341). An adjuvant may also be any ligand suitable for the activation of a pathogen recognition receptor (PRR) expressed in and on dendritic cells, T cells, B cells or other antigen presenting cells. Ligands activating the nucleotide-binding oligomerization domain (NOD) receptor pathway may be suited for the purpose of the vaccine Adjuvants suitable for these ligands may be muramyl dipeptide derivatives. Ligands activating the toll-like receptors (TLRs) may also convene for the purpose of the vaccine. Those receptors are member of the PRR family and are widely expressed on a variety of innate immune cells, including DCs, macrophages, mast cells and neutrophils.

As example of ligands activating TLR, mention may be made, for TLR4 of monophosphoryl lipid A, 3-O-deacytylated monophosphoryl lipid A, LPS from E. coli, taxol, RSV fusion protein, and host heat shock proteins 60 and 70, for TLR2 of lipopeptides such as N-palmitoyl-S-2,3(bispalmitoyloxy)-propyl-cvsteinyl-seryl-(lysil)3-lysine, peptidoglycan of S. aureus, lipoproteins from M. tuberculosis, S. cerevisiae zymosan and highly purified P. gingivalis LPS; for TLR3 of dsRNA, TLR5 of flagellin and TLR7 synthetic compounds such as imidazoquinolines; or for TLR9 of certain types of CpG-rich DNA. Other useful adjuvants for the vaccine may be T helper epitopes.

The vaccines or immunogenic compositions can be formulated into pharmaceutical compositions (also called “medicaments”) for treating an individual infected with HIV, Ebola, Marburg etc. Pharmaceutical compositions are preferably sterile (aspetic) and pyrogen free, and also comprise a pharmaceutically acceptable carrier. Suitable pharmaceutically acceptable carriers include water, saline solutions (e.g. physiological saline), viscosity adjusters and other conventional pharmaceutical excipients or additives used in the formulation of pharmaceutical compositions for use in humans. Suitable pharmaceutical excipients include stabilizers, antioxidants, osmolality adjusting agents, buffers, and pH adjusting agents. Suitable additives include physiologically biocompatible buffers (e.g. tromethamine hydrochloride), chelants (e.g. DTP A, DTPA-bisamide) or calcium chelate complexes (e.g. calcium DTP A, CaNaDTP A-bisamide), or, optionally, additions of calcium or sodium salts (e.g. calcium chloride, calcium ascorbate, calcium gluconate, calcium lactate).

A typical regimen for treating an individual chronically infected with HIV, or other virus, which can be alleviated by a cellular immune response by active therapy, comprises administration of an effective amount of a vaccine composition as described above, administered as a single treatment, repeatedly, with or without enhancing or booster dosages, over a period up to and including one week to about 24 months.

An “immunogenically effective amount” of an essentially inactivated HIV or other pathogen or of an immunogenic composition is one which is sufficient to cause the subject to a specific and sufficient immunological response, so as to impart protection against subsequent HIV or viral exposures to the subject. In this case, an effective amount causes a cellular or humoral response to HIV or pathogen introduced, preferably, a cellular immune response.

The immunogenically effective amount results in the amelioration of one or more symptoms of a viral disorder, or prevents the advancement of a viral disease, or causes the regression of the disease or decreases viral transmission. For example, an immunogenically effective amount refers preferably to the amount of a therapeutic agent that decreases the rate of transmission, decreases HIV viral load, or decreases the number of infected cells, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more. An immunogenically effective amount, with reference to HIV, also refers to the amount of a therapeutic agent that increases CD4⁺ cell counts, increases time to progression to AIDS, or increases survival time by at least 5%, preferably, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more.

It is understood that the effective dosage will be dependent upon the age, sex, health, and weight of the recipient, as well as viral load and CD4⁺ T cell count, kind of concurrent treatment, if any, frequency of treatment, and the nature of the effect desired. In one embodiment, dosage will be tailored to the individual subject, as is understood and determinable by one of skill in the art, without undue experimentation. See Gennaro, 2003.

The efficacy of treatment can be assessed through different means such as, for example, by monitoring the viral load and CD4⁺ T cell count in the blood of an infected subject or by measuring cellular immunity.

The monitoring of the viral load and CD4⁺ T cell count in the blood is carried out by standard procedures. If the vaccine and/or immunogenic composition is efficacious, there should be greater than or equal to one log reduction in viral load, including to less than 10,000 copies/mL of, for example, HIV-R A within 2-4 weeks after the commencement of treatment. If a reduction in viral load of less than 0.5 log is attained, or if, for example, HIV-R A stays above 100,000, then the treatment should be adjusted by either adding or switching drugs. Viral load measurement should be repeated every 4-6 months if the subject is clinically stable. If viral load returns to 0.3-0.5 log of pre-treatment levels, then the therapy is no longer working and should be changed. Within 2-4 weeks of starting treatment, CD4⁺ T-cell count should be increased by at least 30 cells/mm³. If this is not achieved, then the therapy should be changed. The CD4⁺ T-cell counts should be monitored every 3-6 months during periods of clinical stability, and more frequently, should symptomatic disease occur. If CD4⁺ T-cell count drops to baseline (or below 50% of increase from pre-treatment), then the therapy should be changed.

To measure cellular immunity, cell suspensions of enriched CD4+ and CD8⁺ T cells from lymphoid tissues are used to quantify antigen-specific T cell responses by cytokine-specific ELISPOT assay. See Wu S, et al, 1995, 1997. Such assays can measure the numbers of antigen-specific T cells that secrete IL-2, IL-4, IL-5, IL-6, IL-10 and IFN-γ. All ELISPOT assays are conducted using commercially-available capture and detection mAbs (R&D Systems, Inc., Minneapolis, Minn., USA; BD Biosciences Pharmingen, San Diego, Calif., USA). See Wu S, et al, 1995, 1997, supra; Shata M, 2001, supra. Each assay includes mitogen (Con A) and ovalbumin controls.

The “HIV antigen” is the whole inactivated HIV virus, an part thereof or any virus or part thereof introduced which is capable of generating an immune response in a subject. Said immune response can be the production of antibodies or cell-mediated immune responses against the virus. Particularly, “immune response” refers to a CD8⁺ T cell mediated immune response to HIV infection. An immune response to HIV may be assayed by measuring anyone of several parameters, such as viral load, T cell proliferation, T cell survival, cytokine secretion by T cells, or an increase in the production of antigen-specific antibodies (e.g. antibody concentration).

Thus, the immunogenic compositions are useful for preventing HIV infection or slowing progression to AIDS in infected individuals or other viruses. The compositions containing HIV antigen produced from HIV grown in chemically defined, protein free medium and methods of using such compositions can be used to elicit potent Th1 cellular and humoral immune responses specific for conserved HIV epitopes, elicit HIV-specific CD4 T helper cells, HIV-specific cytotoxic T lymphocyte activity, stimulate production of chemokines and cytokines such as—chemokines, IFN-, interleukin 2 (IL-2), interleukin 7 (IL-7), interleukin 15 (IL-15), or α-defensin, and increase memory cells. Such vaccines or immunogenic compositions can be administered via various routes of administration. Such vaccines or immunogenic compositions can be used to prevent maternal transmission of HIV, for vaccination of newborns, children and high-risk individuals, and for vaccination of infected individuals. Such vaccines or immunogenic compositions can optionally include immunomers or an immunostimulatory sequence (ISS) to enhance an immune response against the HIV antigen. Such vaccines or immunogenic compositions can also be used in combination with other HIV therapies, including antiretro viral therapy with various combinations of nuclease and protease inhibitors and agents to block viral entry, such as T20. See Baldwin C, et al, Curr. Med. Chem. 2003; 10: 1633-1642.

The immunogenic compositions when administered to a subject that has no clinical signs of the infection can have a preventive activity, since they can prevent the onset of the disease.

The beneficial prophylactic or therapeutic effect of an HIV (or other viral) immunogenic composition in relation to viral infection, such as HIV infection or AIDS symptoms include, for example, preventing or delaying initial infection of an individual exposed to virus, such as HIV; reducing viral burden in an individual infected with HIV; prolonging the asymptomatic phase of HIV infection; maintaining low viral loads in HIV infected subjects whose virus levels have been lowered via anti-retroviral therapy; increasing levels of CD4 T cells or lessening the decrease in CD4 T cells, both HIV-1 specific and non-specific, in drug naïve subjects and in subjects treated with ART, increasing overall health or quality of life in an individual with AIDS; and prolonging life expectancy of an individual with AIDS. A clinician can compare the effect of immunization with the subject's condition prior to treatment, or with the expected condition of an untreated subject, to determine whether the treatment is effective in inhibiting AIDS.

In one embodiment, the immunogenic compositions are preventive compositions.

The immunogenic compositions may be useful for the therapy of HIV-1 infection or infection by Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Hep A, Hep B, Hep C, the rhino virus, polio, and chickenpox virus or Malaria. While all animals that can be afflicted with HIV-1 or their equivalents can be treated in this manner (e.g. chimpanzees, macaques, baboons or humans), the immunogenic compositions are directed particularly to their therapeutic uses in humans. Often, more than one administration may be required to bring about the desired therapeutic effect; the exact protocol (dosage and frequency) can be established by standard clinical procedures.

For a long time the main obstacle to creating an HIV or deadly virus vaccine has been the high genetic variability of the virus. However, regional hyperthermia offers a solution.

There is a need to provide an active immunotherapy that is capable of overcoming viral immunoavoidance mechanisms and to train the human immune system to perceive the threat/danger of viral infected cells resulting in an immune response against pathogen infected cells wherever they might be located in the body. The approach discussed herein is novel and unique in the world: no one else has yet developed customized immunotherapy using the virus from individual patients and coupled with perfusion hyperthemia.

Immunotherapy is based on the properties of dendritic cells, whose role is to present specific proteins from infectious organisms at their surface, thereby alerting the rest of the immune system. Antigen-presenting cells, (also known as accessory cells) of the mammalian immune system—their main function is to process antigen material and present it on the cell surface to the T cells of the immune system. They act as messengers between the innate and the adaptive immune systems.

Dendritic cells are present in those tissues that are in contact with the external environment, such as the skin (where there is a specialized dendritic cell type called the Langerhans cell) and the inner lining of the nose, lungs, stomach and intestines. They can also be found in an immature state in the blood. Once activated, they migrate to the lymph nodes where they interact with T cells and B cells to initiate and shape the adaptive immune response. At certain development stages they grow branched projections, the dendrites that give the cell its name (dendron being Greek for “tree”). While similar in appearance, these are distinct structures from the dendrites of neurons. Immature dendritic cells are also called veiled cells, as they possess large cytoplasmic ‘veils’ rather than dendrites. In short these cells boost the immune system.

The methodology includes the expansion of dendritic cells of patients in vitro and then treated with the RNA (ribonucleic acid) from the virus that had infected the patient following this immunotherapy is optional hyperthermia treatment. To isolate dendritic cells, blood dendritic cell isolation via a magnetic labeling system was used for the concurrent isolation of plasmacytoid and myeloid dendritic cells from human PBMCs (peripheral blood mononucleated cell) (FIG. 10). The isolation is performed in a two-step procedure. First, B cells and monocytes are magnetically labeled and depleted using a cocktail of CD19 and CD14 MicroBeads. Subsequently, the pre-enriched dendritic cells in the non-magnetic flow-through fraction are magnetically labeled and enriched using a cocktail of antibodies against the dendritic cell markers CD304 (BDCA-4/Neuropilin-1), CD141 (BDCA-3), and CD1c (BDCA-1). The highly enriched cell fraction comprises plasmacytoid dendritic cells, CD1c (BDCA-1) type-1 myeloid dendritic cells (MDC1s), and CD1c (BDCA-1)-CD141 (BDCA-3) bright type-2 myeloid dendritic cells (MDC2s). B cells and monocytes are depleted in advance because a subpopulation of B cells expresses CD1c (BDCA-1), and monocytes express CD141 (BDCA-3) at low levels. This leads to dendritic cells suitable for in vitro induction of primary T cell response.

FIG. 11 provides steps for a method for collecting, culturing, multiplying (expanding) and differentiating, in vitro, stem cells and using such cells for treatment, such as autologous treatment, of patients.

Stem cells can be harvested from adipose tissue, specifically lipoaspirate and peripheral blood. The stem cells can be expanded to a desirable population and then differentiated into T-cells, dendritic cells, NK cells (or other immune cells). The cells can then be exposed to 3D/4D printed molds of desired virus or bacteria, thus generating immune response triggering surface antigen expression or exposing the cells to virus/bacteria or particles thereof. Once expression has occurred, the cells can be infused back into the patient. When infused, the immune system response will be strengthened at least about 2 fold.

Culture media for culturing mammalian cell lines in vitro are known to those skilled in the art and commonly used. In one embodiment, the usual culture media, such as RPMI 1640 Glutamax (Gibco™) supplemented with sodium pyruvate, nonessential amino acids and decomplemented fetal calf serum, are used.

An in vitro method for obtaining activated human plasmacytoid dendritic cells comprises the following steps: an isolated cell line, the cell line is activated so as to obtain activated human plasmacytoid dendritic cells. In one embodiment, the cell line is activated with a virus and/or IL3 and/or CD40. The methods for activating, in vitro, a human plasmacytoid dendritic cell line make it possible to obtain a large number of activated or mature plasmacytoid dendritic cells. This also relates to activated or mature human plasmacytoid dendritic cells obtained from the cell lines (Grouard G. et al., J. Exp. Med., 1997; Cella M. et al., Nat. Immunol., 2000; 1:305-310). The activation or the maturation of the human plasmacytoid dendritic cell lines is therefore carried out according to techniques available to an art worker. The methods make it possible to obtain a large number of activated or mature cells by virtue of the use of human plasmacytoid dendritic cell lines, the cell lines are activated with an enveloped or naked, single-stranded or double-stranded, RNA virus (for example, HIV, HTLV, influenza, mumps, measles, dengue and ebola) or DNA virus (for example, adenovirus, HSV, CMV, EBV), or derivatives thereof (poly-IC), with bacteria (for example, M. tuberculosis) or derivatives thereof (CpG ODN), or with parasites (for example, leishmania) or fungi (for example, Candida albicans). In one embodiment, the activation is carried out in the presence of at least one virus chosen from influenza, HIV, EBOLA and HSV.

The human plasmacytoid dendritic cell lines are activated with stimuli of T lymphocyte origin, which are soluble factors such as cytokines (for example, IL3, GM-CSF or IFNa) or ligands that interact with surface receptors such as the proteins of the TNF family (CD40L or anti-CD40, for example). In one embodiment, the human plasmacytoid dendritic cell lines are activated with IL3 and/or CD40L. The human plasmacytoid dendritic cell lines are activated with IL3, CD40L and a virus. By way of example, the activation or the maturation of the plasmacytoid dendritic cell lines is induced by the addition of virus, of IL3-CD40L or of virus-IL3-CD40L to the culture medium.

One embodiment for activating T lymphocytes, in vitro, comprises the following steps: a) an isolated cell line is provided; b) said cell line of step a) is activated so as to obtain activated human plasmacytoid dendritic cells; c) T lymphocytes are brought into contact with said activated human plasmacytoid dendritic cells of step b). In one embodiment, the cell line of plasmacytoid dendritic cells is activated with a virus, IL3 and/or CD40. This method can be carried out on any type of biological sample comprising T lymphocytes. In one embodiment, it is a human or animal biological sample. The sample can be blood and, for applications of the immunotherapy and cell therapy type, it is generally autologous blood.

A further embodiment provides a method for identifying compounds that activate human plasmacytoid dendritic cells, comprising the steps of a) bringing the compound into contact with the plasmacytoid dendritic cell line: b) detecting the activation of said cell line. The detection of the activation or of the maturation of the human plasmacytoid dendritic cells is carried out according to conventional techniques known to those skilled in the art. In one embodiment, the secretion of at least one molecule chosen from pro-inflammatory cytokines (for example, IL6, TNFa and IFNa), cytokines that orient the immune response (for example, IL12 and IFNa), chemokines (for example, IL-8, RANTES, IP10, MIG, MDC, TARO, I309) and antiviral cytokines (for example, IFNa) is detected. In one embodiment, the secretion of at least one molecule chosen from IL12, TNF, IL6, IL8 and IFNa is detected. In another embodiment, the increase in expression of HLA molecules, of costimulatory molecules (for example, CD40, CD80, CD86), of CD83 and of chemokine receptors (for example, CCR6 and CCR7) is detected.

One embodiment is directed to a pharmaceutical composition comprising at least one cell of a plasmacytoid dendritic cell line that has been treated with RNA (viral or bacterial) or other treatment prior to administration to a subject.

A virus sample is taken before the administration of any antiretroviral treatment. The surfaces of the manipulated dendritic cells (those exposed to virus and/or viral RNA obtained from, for example, the infected patient to be treated) present an increased number of, for example, HIV proteins, which allows them to stimulate the cytotoxic response of a certain type of immune cell called CD8⁺ lymphocytes.

RNA-loaded antigen-presenting cell (APC): the method involves introducing into an APC in vitro (i) derived RNA that includes virus-specific RNA which encodes a cell-surface viral antigenic epitope which induces T cell proliferation or (ii) pathogen-derived RNA that includes pathogen-specific RNA which encodes a pathogen antigenic epitope that induces T cell proliferation. Upon introducing RNA into an APC (i.e., “loading” the APC with RNA), the RNA is translated within the APC, and the resulting protein is processed by the MHC class I or class II processing and presentation pathways. Presentation of RNA-encoded peptides begins the chain of events in which the immune system mounts a response to the presented peptides.

In one embodiment, the APC is an APC such as a dendritic cell or a macrophage. Alternatively, any APC can be used. For example, endothelial cells and artificially generated APC can be used. The RNA that is loaded onto the APC can be provided to the APC as purified RNA, or as a fractionated preparation of a virus or pathogen. The RNA can include poly A+RNA, which can be isolated by using conventional methods (e.g., use of poly dT chromatography). Both cytoplasmic and nuclear RNA are useful. Also useful is RNA encoding defined pathogen antigens or epitopes, and RNA “minigenes” (i.e., RNA sequences encoding defined epitopes). If desired, pathogen-specific RNA can be used

The RNA that is loaded onto APC can be isolated from a cell, or it can be produced by employing conventional molecular biology techniques. For example, RNA can be extracted from blood cells, reverse transcribed into cDNA, which can be amplified by PCR, and the cDNA then is transcribed into RNA to be used. If desired, the cDNA can be cloned into a plasmid before it is used as a template for RNA synthesis. RNA that is synthesized in vitro can, of course, be synthesized partially or entirely with ribonucleotide analogues or derivatives. These can be used, for example, to produce nuclease-resistant RNAs. The use of RNA amplification techniques allow one to obtain large amounts of the RNA antigen from a small number of cells.

It is not necessary that the RNA be provided to the APC in a purified form. For example, the RNA sample is at least about 50%, or about 75%, about 90%, or even about 99% RNA (wt/vol) antigen-presenting cells, including professional APC such as dendritic cells and macrophage. Such cells can be isolated according to procedures available to an art worker.

Any of a variety of methods can be used to produce RNA-containing pathogen preparations. For example, a mammalian cell culture medium such as Opti-MEM or a buffer such as phosphate buffered saline. Pathogen-derived RNA can be produced by sonicating cells containing a pathogenic virus. Other methods for disrupting cells also are suitable, provided that the method does not completely degrade the pathogen-derived RNA. Typically, the RNA preparation has about 10⁶ to about 10 cells/ml; including about 10⁷ cells/ml. As alternatives, or in addition, to sonication, pathogen-derived RNA can be prepared by employing conventional RNA purification methods such as guanidinium isothiocyanate methods and/or oligo dT chromatography methods for isolating poly A⁺ RNA. IVT RNA, synthesized according to conventional methods, can be used in lieu of RNA in pathogen preparations. For example, RNA from a pathogen can be reverse transcribed into cDNA, which then is amplified by conventional PCR techniques to provide an essentially unlimited supply of cDNA corresponding to the pathogen RNA antigen. Conventional in vitro transcription techniques and bacterial polymerases then are used to produce the IVT RNA. As an alternative, the IVT RNA can be synthesized from a cloned DNA sequence encoding a pathogen polypeptide antigen. Methods for identifying such antigens are known in the art; for example, several melanoma peptide antigens have been identified. RNA transcribed in vitro from cDNA encoding identified peptide antigens can serve as pathogen-specific RNA in the invention. As an alternative, RNA can be transcribed from “minigenes” consisting of a portion of the viral antigen cDNA that encodes an epitope. Pathogen-specific RNA can also be produced by employing conventional techniques for subtractive hybridization. For example, an RNA sample from pathogen cells can be used in the subtractive hybridization method to obtain pathogen specific RNA.

If desired, the pathogen-derived RNA can be prepared from frozen or fixed tissues including blood. Although not required, the tissue sample can be enriched for pathogen-specific RNA. Microdissection techniques that are suitable for separating cells. Once cells are separated, RNA can be isolated from the pathogen cells. Conventional in vitro transcription methods then can be used to synthesize the RNA. Other art-known methods for amplifying RNA from a small number of cells, or even a single cell, also can be used

An RNA molecule that encodes a pathogen antigenic epitope can, if desired, be engineered such that it also encodes a cell trafficking signal sequence. Such a chimeric RNA molecule can be produced using conventional molecular biology techniques. The chimeric RNA that is introduced into an APC encodes a chimeric polypeptide, which contains an antigen linked to a trafficking sequence that directs the chimeric polypeptide into the MHC class II antigen presentation pathway. For example, the trafficking sequences employed may direct trafficking of the polypeptide to the endoplasmic reticulum (ER), a lysosome, or an endosome, and include signal peptides (the amino terminal sequences that direct proteins into the ER during translation), ER retention peptides such as KDEL (SEQ ID NO: 1); and lysosome-targeting peptides such as KFERQ (SEQ ID NO: 2), QREK (SEQ ID NO: 3), and other pentapeptides having Q flanked on one side by four residues selected from K, R, D, E, F, I, V, and L. An example of a signal peptide that can be used is the LAMP-1 sorting signal (Wu et al., 1995, Proc. Natl. Acad. Sci. 92:11671-11675; Lin et al., 1996, Cancer Research 56: 21-26). Another example of a signal peptide that is useful is a signal peptide substantially identical to that of an MHC subunit such as class II α or β; e.g., the signal peptide of MHC class II a is contained in the sequence MAISGVPVLGFFIIAVLMSAQESWA (SEQ ID NO: 4). If desired, the signal peptide encoded by the RNA may include only a portion (typically at least ten amino acid residues) of the specified 25 residue sequence, provided that portion causes trafficking of the polypeptide to the ER.

Transfection methods that are suitable for introducing the pathogen-derived RNA into an antigen-presenting cell are known in the art. For example, 5-50 μg of RNA in 500 μl of Opti-MEM can be mixed with a cationic lipid at a concentration of 10 to 100 jpg, and incubated at room temperature for 20 to 30 minutes. Other suitable lipids include LIPOFECTIN™ (1:1 (w/w) DOTMA:DOPE), LIPOFECTAMINE™ (3:1 (w/w) DOSPA:DOPE), DODAC:DOPE (1:1), CHOL:DOPE (1:1), DMEDA, CHOL, DDAB, DMEDA, DODAC, DOPE, DORI, DORIE, DOSPA, DOTAP, and DOTMA. The resulting RNA-lipid complex is then added to 1-3×10⁶ cells, preferably 2×10⁶, antigen-presenting cells in a total volume of approximately 2 ml (e.g., in Opti-MEM), and incubated at 37° C. for 2 to 4 hours. Alternatively, the RNA can be introduced into the antigen presenting cells by employing conventional techniques, such as electroporation or calcium phosphate transfection with about 1-5×10⁶ cells and about 5 to about 50 μg of RNA. Typically, about 5-20 μg of poly A⁺ RNA or about 25-50 μg of total RNA is used.

When the RNA is provided as pathogen preparation, the preparation typically is fractionated or otherwise treated to decrease the concentration of proteins, lipids, and/or DNA in the preparation, and enrich the preparation for RNA. For example, art-known RNA purification methods can be used to at least partially purify the RNA from the pathogen. It is also acceptable to treat the RNA preparation with proteases or RNase-free DNases. Of course, the RNA can be synthesized using art-known nuclease-resistant analogues or derivatives in order to render the RNA less susceptible to ribonucleases.

If desired, RNA encoding an immunomodulator, such as a cytokine or a co-stimulatory factor, can be introduced into the RNA-loaded APCs. The RNA encoding the immunomodulator may be introduced into the APC prior to, simultaneously with, or subsequent to introduction of the pathogen-derived RNA. The methods described herein for introducing the pathogen-derived RNA into the APC also are suitable for introducing into the APC RNA encoding an immunomodulator (e.g., a cytokine or costimulatory factor). If desired, RNA encoding two or more immunomodulators can be introduced into the APC. Typically, about 5-20 μg of each RNA is introduced into the APC, as is described above for pathogen-derived RNA.

The RNA-loaded antigen-presenting cells can be used to stimulate CTL proliferation in vivo or ex vivo. The ability of RNA-loaded antigen-presenting cells to stimulate a CTL response can be measured or detected by measuring or detecting T-cell activation, for example, in a conventional cytotoxicity assay. The cytotoxicity assay entails assaying the ability of the effector cells to lyse target cells. If desired, the target cells can be RNA-loaded APCs produced (i.e., APCs that present an RNA-encoded cell-pathogen antigenic epitope that induces T cell proliferation). The commonly-used europium release assay can be used to assay CTL sensitization. Typically, about 5-10×10⁶ target cells are labeled with europium diethylenetriamine pentaacetate for 20 minutes at 4° C. After several washes 10⁴ europium-labeled target cells and serial dilutions of effector cells at an effector:target ratio ranging from 50:1 to 6.25:1 are incubated in 200 μl RPMI 1640 with 10% heat-inactivated fetal calf serum in 96-well plates. The plates are centrifuged at 500×g for 3 minutes and the incubated at 37° C. in 5% CO₂ for 4 hours. A 50 μl aliquot of the supernatant is collected, and europium release is measured by time resolved fluorescence (Volgmann et al., J. Immunol. Methods 119:45-51, 1989).

In an alternative method for detecting CTL sensitization, an increase in cytokine secretion by the CTL is detected, relative to the level of cytokine secretion prior to contacting the CTL with an RNA-loaded APC. In situ hybridization assays, such as ELISPOT assays, can be used to detect secretion of cytokines such as TNF-α and/or γ-interferon.

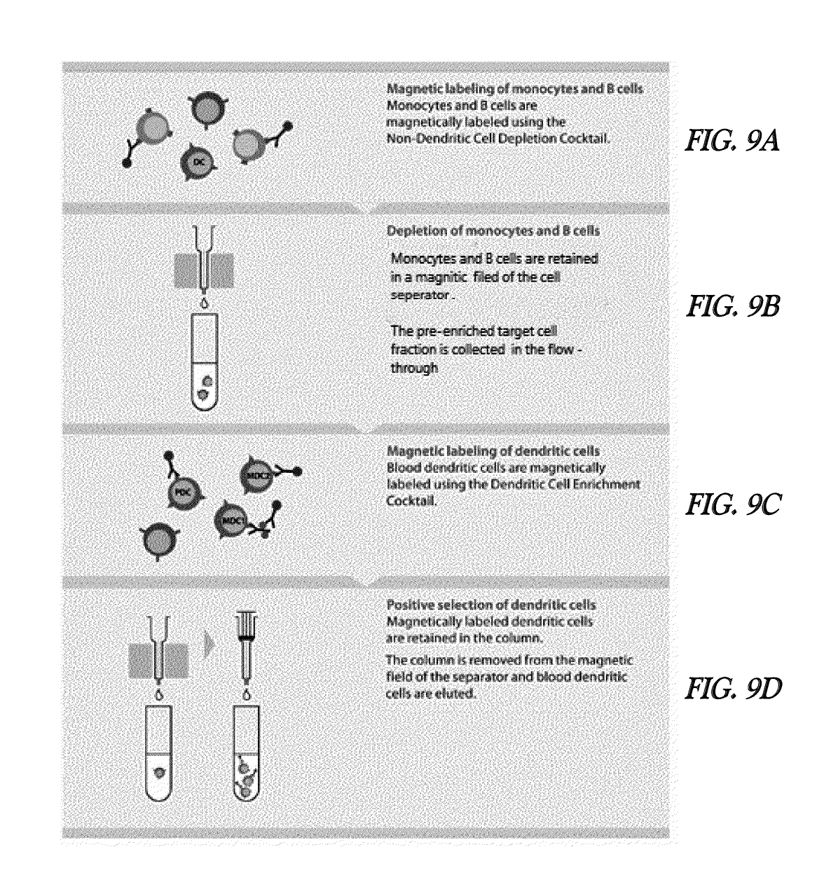

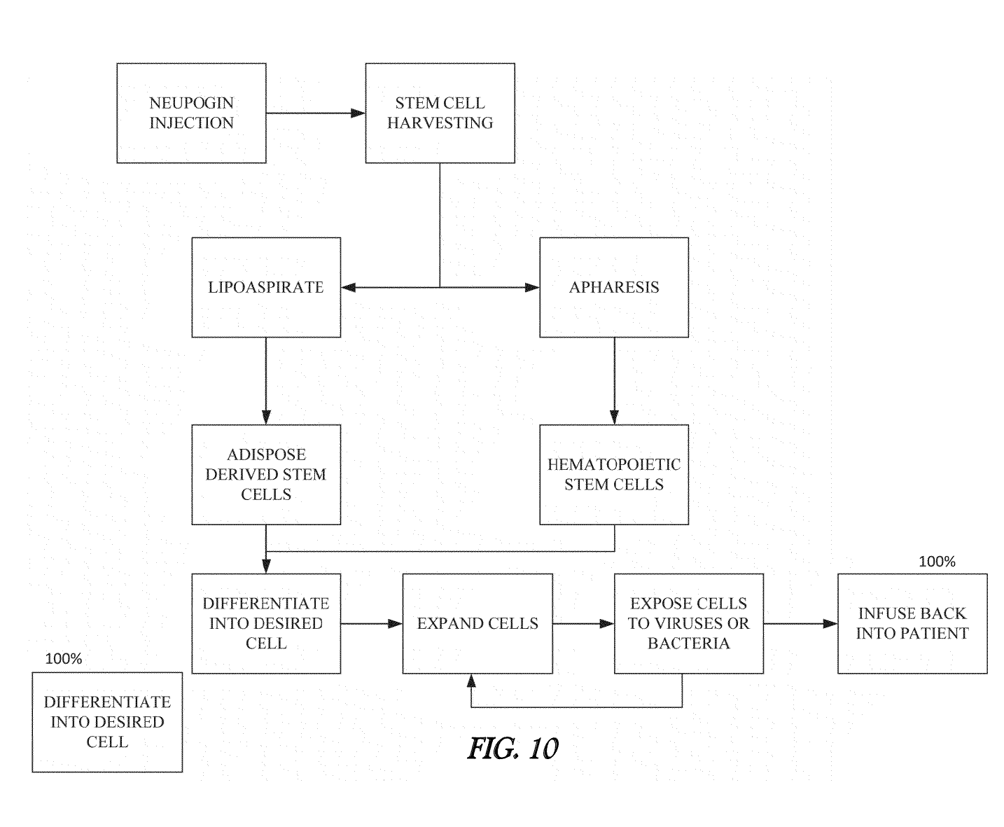

FIG. 9 depicts methods for assessing CD8⁺ T cell antiviral function in acute HIV-1 infection. Four drawings are used to assess CD8⁺ T cell function. Two drawings measure the ability of primary CD8⁺ T lymphocytes to inhibit HIV-1 virus replication (using full-length HIV-1 transmitted/founder infectious molecular clones) via co-culture and through soluble mechanisms. Soluble cytokine receptors regulate inflammatory and immune events by functioning as agonists or antagonists of cytokine signaling. (In pharmacology the term agonist-antagonist is used to refer to a drug which exhibits some properties of an agonist (a substance that fully activates the neuronal receptor that it attaches to) and some properties of an antagonist (a substance that attaches to a receptor but does not activate it or if it displaces an agonist at that receptor it seemingly deactivates it thereby reversing the effect of the agonist). As such, they act within complex receptor systems that include signaling receptors, non-signaling decoy receptors, receptor-associated proteins, and soluble receptor antagonists. Soluble cytokine receptors can be generated by several mechanisms, which include proteolytic cleavage of receptor ectodomains, alternative splicing of mRNA transcripts, transcription of distinct genes that encode soluble cytokine-binding proteins, release of full-length receptors within the context of exosome-like vesicles, and cleavage of GPI-anchored receptors (GPI anchor) is a glycolipid that can be attached to the C-terminus of a protein during posttranslational modification. It is composed of a phosphatidylinositol group linked through a carbohydrate-containing linker (glucosamine and mannose glycosidically bound to the inositol residue) and via anethanolamine phosphate (EtNP) bridge to the C-terminal amino acid of a mature protein. The two fatty acids within the hydrophobic phosphatidyl-inositol group anchor the protein to the cell membrane. Furthermore, the role of soluble cytokine receptors in regulating host defense mechanisms is evidenced by viruses that encode soluble homologues of mammalian receptors and thereby evade innate host immune responses via the sequestration of essential cytokines.

Although they are known primarily for their capacity to kill infected cells, CD8(+) T cells elaborate a variety of effector mechanisms with the potential to defend against infection. Microbes use multiple strategies to cause infection, and the nature of the pathogen host interaction may deter mine which CD8(+) T cell effector mechanisms are required for immunity.

After receiving multiple subcutaneous injections of these dendritic cells, patients will experience a significant increase in CD8⁺ lymphocyte activity. It is evident that CD8(+) T cells contribute to resistance against intracellular infections with certain viral, protozoan, and bacterial pathogens.

Every patient's immune system has the ability to fight a deadly virus including HIV. With deadly viruses, including HIV, the immune system has to be reactivated using use cells created with memory that seek and destroy any new infected cells that appear.

One prophetic example of a therapy scheme for hyperthermic treatment of a body fluid infected with a pathogen is provided below. The prophetic example is provided as an exemplary schema and accordingly the target treatment temperatures provided for differing measured viral loads will vary according to the pathogen treated. In the prophetic example, the pathogen includes one or more of Crimean Congo hemorrhagic fever, Dengue fever, Dengue fever-febrile, Dengue feve-defevrescent, Dengue hemorrhagic fever, Dengue hemorrhagic fever-febrile, Dengue hemorrhagic fever-defevrsescent, Ebola, Hepatitis C virus, HIV, Lassa virus, Rift Valley fever or Sin Nombre. The exemplary therapy scheme for hyperthermic treatment as described herein includes a graduated series of treatment temperatures that vary according to measured viral loads of a body fluid. The exemplary therapy scheme is provided below in table form.

| | |
|---|---|
| Measured Viral Load (in units | Body Fluid Target |
| of pathogen copies per | Treatment Temperature |
| milliliter of body fluid): | (in degrees Fahrenheit) |

| | | |
|---|---|---|
| 50 | copies/ml | 102 degrees F. |
| 10k-30k | copies/ml | 105 degrees F. |
| 100k | copies/ml | 110 degrees F. |

As shown, the target treatment temperature (and the heating provided by the hyperthermic heat exchanger) increases with higher measured viral loads. In one example, the hyperthermic treatment assembly 100 is continually operated and the target treatment temperature regulated (up or down) according to the measurement of the viral load. For instance, as the measured viral load decreases the hyperthermic heat exchanger 112 (controlled by the body fluid controller 116) gradually decreases the target treatment temperature to the corresponding temperature stored for a particular therapy scheme.

In a similar manner to the prophetic example provided above the hyperthermic treatment assembly 110 may include or receive one or more therapy schemes for each of a variety of pathogens, each scheme with differing target treatment temperatures associated with measured viral loads. Example pathogens include, but are not limited to, Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Malaria, Hep A, Hep B, Hep C, the rhino virus, polio, and chickenpox virus or HIV. The temperature of the body fluid of the subject (e.g., blood) is raised with the methods and devices described herein to a temperature (including a range of temperatures) and a duration of elevated temperature sufficient to reduce the viral load of the subject by 40 percent or more. Optionally, before, after or during hyperthermic treatment, the subject is treated with a follow up therapy of immunotherapy to further treat the subject for the pathogen.

### Additional Embodiments

Adipose cells can be cultured and directed to generate stem cells populations by culturing in a basal medium such as DMEM/F12, antibiotics, antimycotics, nutrients (amino acids, fatty acids, minerals), growth factors, and/or hormones. In one aspect, the ingredients comprise a supplemented defined medium including DMEM/F12, L-glutamine, an antibiotic, an antimycotic, ITS+3 (such as Sigma 1-2771; an insulin, transferrin, and selenium composition), a fatty acid supplement, non-essential amino acids, ascorbic acid 2-phosphate (ASAP), PDGF-BB, EGF, SCGF-β, TNFα, IL-3, beta-estradiol, progesterone, dexamethasone, and hydrocortisone. In one aspect, the protein and hormone supplements are human. One of ordinary skill in the art will appreciate that media other than DMEM F12 could be modified for use as well. In another aspect, serum can be added. In one aspect, the serum is used at low concentrations. In one aspect, the serum is used at concentrations as low as about 0.5%. In one aspect, the serum is human serum.

The cells can be plated onto culture dishes which have been coated with at least one adhesion factor. In one aspect, other coatings are applied to the tissue culture dish. In one aspect, adhesion factors are added to the medium.

In one embodiment, a base medium for use in invention is serum-free and can be supplemented with differentiation-inducing factors and agents to induce and support differentiation of adipose tissue-derived stem cells. In one aspect, the medium is useful for inducing differentiation of adipose tissue-derived stem cells along differentiation pathways selected from the group consisting of adipose, chondrogenic, and osteogenic. One of ordinary skill in the art would appreciate that the base medium of the invention can be supplemented with various growth factors, hormones, and other agents useful in inducing and maintaining differentiation of various cell types, including, adipogenic, chondrogenic, and osteogenic. It is also known that ASCs can be induced to differentiate along neural and myogenic lineages (cardiac, skeletal, and smooth muscle). It is anticipated that the base medium of the invention will be useful for inducing differentiation of such cell types when supplemented with the appropriate differentiation-inducing agents. A serum-free or low serum medium for culturing adipose tissue-derived stem cells wherein said cells retain the ability to differentiate, said medium comprising a basal medium and additional ingredients.

Human brown adipose tissue stem cells/line expresses the markers CD9, SSEA4, CD44, CD90, CD166, CD73, but not CD14, CD34, CD45 or STRO-1. In another embodiment, the isolated human brown adipose tissue stem cells/line expresses the genes UCP1, PPARGC1A, NRF1, FOXC2, CREB1, SIRT3, and WNT5A (REFX). In still another embodiment, the isolated human brown adiposet issue stem cells/line is capable of differentiating into osteoblasts, chondrocytes, and adipocytes.

A population of adipose tissue stem cells (such as metabolically active brown adipose tissue stem cells) can be isolated from a subject, such as a new born. The stem cell population, termed “brown adipose derived stem cells” (BADSCs) can (1) be expanded in vitro; (2) exhibit multi-lineage potential; and (3) functionally differentiate into adipocytes (such as metabolically active brown adipocytes). Such a stem cell population can offer new cell-based therapies to restore and enhance energy homeostasis in vivo for the treatment of obesity and related metabolic disorders. These stem cells also are a useful tool for studying adipose tissue biology.

In order to identify a stem cell population within adipose depots (such as newborn mediastinal brown adipose depots), explants were generated and plated into tissue culture plates. Adherent cells were successfully derived from the brown adipose tissue explants, these primary cell cultures were fed every 3 days in media comprising DMEM low glucose, lx Glutamax, 1×NEAA, and 10% platelet lysate. In order to define a clonal population of cells, cell lines were derived by single-cell plating in 96 well plates. Confluency was reached at 6 days and the cells exhibited Mesenchymal Stem Cell (MSC)-like morphology. Mediastinal brown adipose tissue from a 56 year old and an 80 year old were also isolated and charaacterized for comparison with the newborn mediastinal brown adipose tissue. The decrease in the brown adipose tissue is evident; however still present in all subjects.

Growth kinetics of the clonal cell population demonstrated that the population could be propagated for greater than 90 passages. Karyotyping of the clonal cell population at passage 7 demonstrated normal diploid cells without chromosomal aberrations. BADSC, flow cytometry, and immunocytochemistry were used. The neonate BADSC were found to exhibit characteristics that are similar to other MSCs but are not identical. For instance, the neonate BADSC were positive for CD9, SSEA4, CD44, CD90, CD 166, and CD73, but were negative for hematopoietic markers CD14, CD34, and CD45. Furthermore, the neonate BADSC did not express STRO-1, which has been previously found to be expressed in mesenchymal stem cellsderived from various tissues. An analysis of the gene expression profiles of passage 2 neonate BADSC demonstrate that the neonate BADSC have a distinct gene expression profile, in comparison with white adipose derived stem cells. The genes whose expression is enriched in BADSC include pro-brown adipose selective genes such as, for example, CREB1, DI02, IRS1, MAPK14, NRF1, FOXC2, PPARD, PGC1-A, PGC1-B, PRDM16, SRC, UCP1, and WNT5A. The cells also express higher levels of anti-white adipose tissue genes, such as GATA2, KLF2, and KLF3. Expression of these pro-brown selective genes can distinguish brown adipose derived stem cells from stem cells derived from white adipose depots. Passage 2 neonate BADSC were induced to differentiate into osteo, chondro, white, and brown adipogenic cell lineages to determine multi-lineage potential. After three weeks of induction the cells demonstrated the ability to differentiate into osteoblasts, chondrocytes, and adipocytes. When induced to differentiate under osteogenic promoting conditions, the cells formed a mineralized matrix, which was confirmed by alizarin red staining; immunocytochemistry staining of osteocalcin, and RT-PCR analysis for osteopontin, osteonectin, and alkaline-phosphatase, further confirmed differentiation. Chondrogenic differentiation was confirmed by alcian blue staining for sulfated proteoglycans on induced cell pellets. RT-PCR confirmed expression of collagen 2 A, biglycan, and A6, which are markers of chondrogenic differentiation. White adipogenic differentiation was confirmed by Oil Red O staining of lipid droplets. Immunocytochemistry confirmed expression of FABP4, and RT PCR confirmed expression of FABP4, LPL, and PPARy, which are markers of adipogenic differentiation. Real-time qPCR of neonatal brown adipose-differentiated cells demonstrated upregulation of UCP1, elongation of very long chain fatty acids like-3 (ELOVL3) and peroxisome proliferator-activated receptor γ1-α (PGC1A), a major regulator of mitochondrial biogenesis, compared to non-FNDC5 differentiated cells. Conversely, leptin—a gene associated with white fat development—is down regulated in brown-adipose differentiated cells. Higher levels of expression of these brown adipocyte marker genes are consistent with a mature brown adipocyte fate. These findings demonstrate that brown adipose depots from newborns are a source of stem cells that have unique properties than stem cells found in adult brown adipose depots, subcutaneous adipose and visceral adipose depots, and have the ability to differentiate into multiple cell types. BADSC in general can be grown on scaffolding.

In addition, various BADSC lines were immortalized by transfection. In one embodiment a BADSC 150 line was used. Five different plasmid constructs were created, These were a 7286 bp construct termed Blas-T, encoding an EEF1 promoter driving TERT expression, a 4868 bp construct termed Blas-B, encoding an EEF1 promoter driving BMI-1 expression; an 8348 bp construct termed Blas-B-F-T, encoding an EEF1 promoter driving both BMI1 and TERT expression, an 8348 bp construct termed pBlas-BIT encoding an EEF1a1 promoter driving both TERT and BsrS2, and a 16,243 bp construct termed pUCP1-CP-BfT encoding an EEF1A1 promoter driving expression of BMI1 and TERT and UCP1 promoter driving expression of a reporter gene cherry picker. The transfection used Lipofectamine LTX and PLUS reagent. In a second embodiment, the transfection used Fugene HD, Xfect Adult Stem Cell Transfection Reagent and Lipofectamine LTX with PLUS reagent according to manufacturers recommended protocol. Each reagent was tested for efficiency. Fugene HD was the least toxic to the cells and resulted in the highest transfection efficiency for BADSC150 primary cells transfected with construct Blas-BFT encoding EEF1 promoter driving expression of BM11 and TERT (BM11-TERT FMDV2-self processing polypeptide and TERT-BMI1 FMDV2-self processing polypeptide). Because the two orientations of the self-processing polypeptides will function with differing efficiencies depending upon the cell line, both forms were tested in any given experiment. Under EEF1 A1 control, all four resistance options were available: pUNO-hpf Hygromycin; pUNO-pur Puromycin; pUNO-zeo Zeocin; and pUNO-bla Blasticidin

The following single reporter systems were used: *mCherry alone; *NanoLuc (NL) alone; and * Secreted NanoLuc (sNL) alone. The following combination Reporter systems were also used: CP1-NL FMDV2-self processing polypeptide; CP1-sNL FMDV2-self processing polypeptide; NL-CP1 FMDV2-self processing polypeptide; and sNL-CP1 FMDV2-self processing polypeptide

In other experiments, the EEF1 promoter was also replaced with pro-brown (i.e. PRDM16, PGC-1a, C/EBPJ3, Plac8 and UCP-1) and pro-white specific genes (i.e. PPARy, C/EBPa, and AKT-1) used with different combinations of the reporter systems. All of these constructs contain EEF1 promoter driving expression of BMI1 and TERT. EEF1A1 is driving the expression of Bmi1 and Tert1. This construct is used to immortalize the cells. UCP1 is driving the expression of CherryPicker, chimeric membrane-anchored CherryPicker fluorescent protein, which can be monitored via fluorescence microscopy and be captured on magnetic beads using a specific antibody. In this embodiment, BADSC150 cells with this construct are immortal and CherryPicker is only activated when UCP1 is induced. The induction of UCP1 can be accomplished by exposing this cell population to either naturally occurring or synthetic small molecules that up-regulate brown adipose tissue specific genes. In another study CherryPicker is replaced with secreted nanoluc (sNL). In this construct, sNL is synthesized only when the promoter is turned “ON”. In one embodiment, the promoter is UCP1. Since sNL is secreted from the cell and into the cell culture media, one is able to assay the media for levels of sNL for a quantitative analysis of how efficacious the compound is for inducing UCP1 to turn “on” and in turn produce sNL. This system allows for high throughput screening of thousands of small molecules at different concentrations at one time. All combinations of constructs also have been transfected into white adipose stem cells for the purpose of identifying molecules that convert white fat into brown or increase metabolic activity. This cell line, in tandem with its reporter systems, permits for the effective study of the kinetics of brown fat biology; specifically, the mechanism underlying UCP1 up-regulation and increased metabolic activity. This human brown fat stem cell line is valuable due to the fact that the amino acid composition of mouse and human UCP1 is less than 80%, thereby rendering mouse brown fat cell lines inadequate for identifying compounds that activate brown fat specific genes. In addition, a population of immortal implantable BADSC would provide a population of brown adipose tissue that can help regulate metabolism in humans. This technology also provides a cell culture plate comprising human neonatal brown adipose derived cells. The plate can be, for example, a multi-well plate. One or more wells of the cell culture plate can be seeded with human neonatal brown adipose derived cells. The cells can be differentiated brown fat cells. The cells also can be immortalized. The cell culture plates are useful for screening drug compounds by contacting the cells with a candidate drug compound and observing the effect of the drug compound on the human neonatal brown adipose derived cell.

Adipose cells differentiated to form mesenchymal stem cells, chondrocytes, cardiomyocytes, hematopoieticstem cells, skeletal muscle cells, pancreatic cells, lung cells, intestinal cells, or liver cells.

Ppluripotent stem cells (iPS cells) are pluripotent cells originally isolated from somatic cells of the body reprogrammed by genetic and non-genetic approaches (for review see Amabile and Meissner, 2009). While iPS cells share many characteristics of ES cells including the ability to be differentiated in vitro to cells of all three germ layers, they are not identical. Genetic and epigenetic differences between these two cell types have been reported in the literature and these differences may contribute to altered differentiation efficiencies when subjected to in vitro differentiation protocols. ES cells as well as iPS cells serve as an excellent in vitro system for studying differentiation events and as unlimited source for generating various specialized cell types in large quantities for basic research, drug screening and regenerative therapeutic applications.

Protocols to induce a certain germ layer cell type and subsequent definitive tissue types from human pluripotent stem cells, which includes human induced pluripotent stem cells, are numerous, diverse and currently not standardized. They commonly involve differentiation using three major categories of protocols (for a review see Murry and Keller, 2008):

First, based on co-culture of pluripotent stem cells with other cell types such as feeder cells (e.g. D'Amour et al., 2005, Perrier et al., 2005) or a somatic cell type (e.g., induction of cardiomyocytes via co-culture with murine endoderm-like cell lines (Mummery et al., 2007); in medium conditioned by the feeder cells, which induces a certain germ layer fate (Schulz et al., 2003) or alternatively with the addition of factors (D'Amour et al., 2005);

Second, based on adherent culture as monolayers with or in the absence of serum and including the addition of morphogens (Nat et al., 2007; Chambers et al., 2009). Monolayer cultures of pluripotent stem cells can be achieved by dissociating pluripotent stem cells to single cells and then plating those single cells on a coated or uncoated culture surface. Typically, pluripotent stem cells are plated onto culture surfaces that have been pre-coated with extracellular matrix proteins or synthetic peptides that promote the attachment and survival of pluripotent stem cells. Common proteins and peptides that support this attachment and survival are generally known to those in the field and include for example Matrigel™, vitronectin, E-Cadherin, and laminin. Synthetic peptides that can serve as substrates for pluripotent stem cells are also known to those in the field and can include for example integrin-binding RGD peptides. Additionally, cells can be seeded onto synthetic or biological scaffolds, including artificial organ scaffolds or de-cellularized organs or tissues. Generally, single cells are plated at a known density yielding a monolayer of a defined confluence. There is evidence that cell plating densities can affect cultured cells by influencing cell growth, death, and differentiation. For example, plating efficiency of human pluripotent stem cells is improved if single cells are plated at higher density or if the cells are maintained as clumps where localized areas of high density can improve cell survival. Many protocols for the differentiation of pluripotent stem cells to more specified lineages require plating of pluripotent stem cells at a particular confluence. Confluence is typically assessed by the user by visually assessing the percentage of the culture surface covered by the adherent cells. For example, a 50% confluent culture would appear to have adhered cells covering half of the area of the culture surface. A 100% confluent culture would appear to have cells covering the entire culture surface. Confluence therefore does not indicate a particular number of cells given that cells can be of different size (the same number of smaller cells will cover less area than larger cells) or may spread out on a culture surface to different degrees. Pluripotent stem cells can also be plated as clumps or aggregates of 2 or more cells adhered to each other. These clumps also require similar attachment substrates to those required for single pluripotent stem cells. These clumps are typically multilayered and confluence of these cultures is assessed again by estimating the percentage of the culture surface that is not covered by these adhered clumps. Cultures can also be stacked on top of one another to create multilayered or 3-dimensional cultures. In this type of culture system, cells from one monolayer can either be directly in contact with the adjacent monolayer, or the monolayers can be separated from each other by a matrix or other biological or physical barrier;

Third, based on the formation of 3-D aggregates called embryoid bodies (EBs). Cells in the EBs are multipotential, with the propensity to develop into cells of any of the 3 germ layers (endoderm, mesoderm or ectoderm) (Odorico et al., 2001). Usually morphogens are also added either directly at the time of EB formation to serve as inductive cues or at a later time-point (e.g. after plating of the EBs) to selectively support survival of or differentiation to the desired cell lineage (see embryonic stem cell protocols). EBs can be cultured in suspension, for example in ultra-low adherent culture plates or in bioreactors, or they can re-adhered to a culture surface.

Media formulations used in the 3 different categories of protocols above consist of a variety of media components, additives, and supplement mixtures.

The cultures derived from pluripotent stem cells even under the most defined conditions, are inherently heterogeneous, consisting of cell types of different lineages and at different stages of development. Heterogeneity may be explained by intrinsic cell-to-cell signaling and the variations in the time points used when manipulating the cells in some of the protocols. One solution that has been applied to increase the percentages of the desired cell type that are being induced is the use of morphogens like cytokines or growth factors as additives to the medium. This heterogeneity also has its advantages.

There are several methods for culturing human pluripotent stem cells including the use of specialized media with (feeder-dependent) or without (feeder-free) co-culture with mouse or human irradiated fibroblasts. Several home-made and commercial media have been developed to promote the maintenance of the pluripotent state in human pluripotent stem cells including KO-DMEM+Knock Out Serum Replacer (KOSR), conditioned medium from irradiated feeder cells, mTeSR™ 1 (STEMCELL Technologies, Inc., Cat #05850, 2008), TeSR™-E8™ (STEMCELL Cat #05840, 2012), Essential-8™ (Life Technologies, Inc., Cat #A14666SA, 2012) and others.

Another common approach to control for heterogeneity is the use of selection strategies to obtain the desired cell types, such as mechanical selection or promotion of selective survival using certain media supplements and factors. Mechanical selection can be very tedious and also hardly gives rise to an entirely pure population of desired cell types.

There is some evidence in the literature that the osmolality of the culture medium influences cell proliferation, survival and differentiation. For example, the osmolality of mTeSR™ 1 medium was adjusted to a higher osmolality of 340 mOsm/kg compared to more standard osmolality of 290-330 mOsm/kg used in most cell culture media to better maintain the undifferentiated state of the cells (Ludwig et al., 2006). On the other hand, differentiated cell types such as primary neurons isolated from the CNS survive better in medium with low osmolality (230-280 mOsm/kg) compared to standard osmolality (Brewer et al., 1993; Brewer and Price 1996; Kivell et al., 2000). The available information suggests that a specific osmolality is either effective for maintaining cells in the undifferentiated state, promoting survival or maintaining already differentiated cells or mature cells in the differentiated state.

The controlled differentiation of human pluripotent cells into pure or highly enriched population neural progenitorcells and subsequent differentiation of these cells into the 3 cell types of the central nervous system (CNS): neurons, astrocytes and oligodendrocytes without any additional selection procedure would be highly desirable in the field since all these cell populations would To summarize, the field is lacking a standardized media formulation(s) and protocol(s) to induce the 3 germ layers and subsequently more specialized cell types derived thereof in a short period of time. The field also suffers from the lack of standardized protocols which are easy to reproduce in different labs and operator-independent. Furthermore, the field suffers from a lack of formulation(s) and protocol(s) that allow for efficient differentiation to the 3 germ layers or to a specific germ layer from pluripotent stem cells which are cultured under varying maintenance or pluripotency culture conditions.

With regards to ectoderm differentiation, culturing the dissociated cells comprises culturing the dissociated cells in a microwell device for about 24 hours to form aggregates and continuing the culture in the microwell device for more than 24 hours in the culture media followed by releasing the aggregates and adhering onto coated culture dishes and culturing in the culture media for at least 1 day. In one embodiment, the aggregates are cultured in the microwell device for up to 14 days, optionally 5-6 days, prior to releasing the aggregates and adhering onto the coated culture dishes. In another embodiment, the aggregates are cultured in the microwell device for up to 11 days.

In another embodiment, for ectoderm differentiation, culturing the dissociated cells comprises culturing the dissociated cells in the culture media in a microwell device for about 24 hours to form aggregates, releasing the aggregates from the microwell device, followed by culturing the released aggregates in suspension in the culture media for at least 1 day, dissociating and adhering the aggregates onto coated culture dishes and culturing in the culture media for at least 1 day. In one embodiment, the cells are cultured in suspension for up to 14 days, optionally 5-6 days, before dissociating and adhering the aggregates onto coated culture dishes.

For differentiating to endoderm progenitor cells, the cells are cultured in (b) for 16-60 hours in a microwell device, in suspension or adhered to a culture plate. In another embodiment, for differentiating to endoderm progenitor cells, the cells are cultured in (b) for about 24 hours in a microwell device, in suspension or adhered to a culture plate. In yet another embodiment, for differentiating to endoderm progenitor cells, the cells are cultured in (b) for about 48 hours in a microwell device, in suspension or adhered to a culture plate. Differentiating the cells in (c) comprises dissociating the cells of b) and plating the cells onto coated culture dishes and culturing for at least 1 day in the culture media to produce germ layer progenitor cells culturing the dissociated cells in b) comprises culturing the dissociated cells from a) in suspension in the culture media for at least 1 day followed by dissociating the cells and adhering onto coated culture dishes and culturing in the culture media for at least 1 day. In one embodiment, the cells are cultured in suspension for up to 14 days, optionally 5-6 days, prior to dissociating and adhering onto the coated culture dishes. Culturing the dissociated cells in b) comprises culturing the dissociated cells from a) in a microwell device for at least 16 hours to form aggregates and continuing the culture in the microwell device in the culture media for up to 60 hours, prior to differentiating the cells in (c). The pluripotent stem cells have been maintained at an osmolality of 260-310 mOsm/kg prior to (a). In such embodiments (b) comprises culturing the cells in a media of higher osmolality, such as 330-550 mOsm/kg and (c) optionally comprises culturing the cells of (b) in media of lower osmolality, such as 260-360 mOsm/kg, optionally 260-280 mOsm/kg. culturing the dissociated cells in b) comprises culturing the dissociated cells from a) in the culture media in a microwell device for at least 16 hours to form aggregates, releasing the aggregates from the microwell device, followed by culturing the released aggregates in suspension in the culture media for up to 60 hours, prior to differentiating the cells in (c). Culturing the dissociated cells in b) comprises culturing the dissociated cells from a) in suspension in the culture media for at least 16 hours followed by dissociating the cells and adhering onto coated culture dishes and culturing in the culture media for up to 60 hours, prior to differentiating the cells in (c). Culture media for (b) comprises Dulbecco's minimal essential medium (DMEM) and optionally, further comprises vitamins, trace elements, selenium, insulin, lipids, b-mercaptoethanol, non-essential amino acids, antibiotics, bFGF, B27, N2 or mixtures thereor. The culture media for step (b) for endoderm induction comprises a mixture of DMEM and F-12 and optionally further comprises vitamins, salts, trace elements, selenium, insulin, lipids, proteins, amino acids, TGF-beta, FGF2, or mixtures thereof. In another embodiment, the culture media comprises the components. The stem cells are mammalian pluripotent stem cells, optionally, human pluripotent stem cells. In another embodiment, the pluripotent stem cells are induced pluripotent stem cells. Aggregates or clusters comprise embryoid bodies. In an embodiment, the embryoid bodies comprise 10 to 20,000 cells, optionally 500 to 20,000 cells. The culture medium is 260 to 280 mOsm/kg for use in inducing or enriching for ectodermal progenitor cells when the dissociated cells are first cultured in the microwell device and/or in suspension. In another embodiment, the osmolality of the culture medium is 270 to 320 m mOsm/kg for use in inducing or enriching ectodermal progenitor cells when the dissociated cells are plated directly onto coated culture dishes. The osmolality of the culture media is above 280 mOsm/kg, optionally 290 to 550, or 290-340 mOsm/kg, for inducing or enriching for endodermal and/or mesodermal progenitor cells when the dissociated cells are first cultured in the microwell device and/or in suspension. In another embodiment, the osmolality of the culture medium is above 320 mOsm/kg, optionally 320 to 340 mOsm/kg, for inducing or enriching for endodermal and/or mesodermal progenitor cells when the dissociated cells are plated directly onto coated culture dishes.

Also provided herein is a method of maintaining single mesodermal and/or endodermal progenitor cells in culture media with an osmolality of 290-340 comprising generating mesodermal and/or endodermal progenitor cells according to the methods described herein; dissociating the mesodermal and/or endodermal progenitor cells from the adhered cultures; and plating and culturing said progenitor cells, optionally in culture media having an osmolality of 260-360 mOsm/kg, optionally 320-340. In an embodiment, the mesodermal and endodermal progenitorcells are further differentiated to form mesenchymal stem cells, chondrocytes, cardiomyocytes, hematopoieticstem cells, skeletal muscle cells, pancreatic cells, lung cells, intestinal cells, or liver cells. The osmolality of the culture media in (b) is 320-550 mOsm/kg for generating a population of enriched endodermal progenitor cells.

In yet another embodiment, the osmolality of the culture media in (b) is 350-450 mOsm/kg for generating a population of enriched endodermal progenitor cells. In yet a further embodiment, the osmolality of the culture media in (b) is about 365 mOsm/kg for generating a population of enriched endodermal progenitor cells. In yet another embodiment, the culture medium is 320 to 550 mOsm/kg for use in inducing or enriching for endodermal progenitor cells when the dissociated cells are first cultured in the microwell device and/or in suspension. In another embodiment, the osmolality of the culture medium is 320-550 mOsm/kg for use in inducing or enriching endodermal progenitor cells when the dissociated cells are plated directly onto coated culture dishes.

In some embodiments, the pluripotent stem cells are first cultured in a medium that supports the maintenance of undifferentiated pluripotent stem cells where that culture medium has an osmolality below 320 mOsm/Kg. Accordingly, in one embodiment, the pluripotent stem cells used to induce or enrich for endodermal progenitor cells have been maintained in a culture media having an osmolality of 260-310 mOsm/kg prior to dissociating the cells. In one embodiment, this culture medium comprises a mixture of DMEM and F-12 and optionally further comprises vitamins, salts, trace elements, selenium, insulin, lipids, proteins, amino acids, TGF-beta, FGF2, or mixtures thereof. In another embodiment, this culture medium is TeSR™-E8™, Essential 8™, or E8.

In yet another embodiment, the pluripotent stem cells are first cultured in a medium that supports the maintenance of undifferentiated pluripotent stem cells where that culture medium has an osmolality above 320 mOsm/Kg. In one embodiment, this culture medium is mTeSR™ 1, TeSR™ 2, mouse embryonic fibroblast (MEF) conditioned medium, or DMEM+(KOSR). The medium used in (b) to raise the osmolality of culture for endoderm differentiation is any medium that supports the survival of mammalian cells, that medium having an osmolality greater than 330 mOsm/Kg. In one embodiment, the medium is STEMdiff™ Neural Induction Medium (STEMCELL Technologies, Inc. Cat #05831, 2011) where the osmolality has been adjusted with compounds, for example, sodium chloride to be greater than 330 mOsm/Kg.

In one embodiment, the osmolality of the culture medium is manipulated through the addition of a concentrated supplement. In an embodiment, the concentrated supplement comprises a physiological diluent and a salt, such as sodium chloride and optionally further comprises a protein. In an embodiment, the protein is albumin, optionally albumin is derived from a recombinant source. In an embodiment, the albumin is human recombinant albumin. In an embodiment, the culture medium used to differentiate the human pluripotent stem cells to definitive endoderm is STEMdiff™ Definitive Endoderm (STEMCELL Technologies, Inc. Cat #05110) whereby the protocol is followed according to manufacturer's instructions. In another embodiment, the culture medium used to differentiate the human pluripotent stem cells to definitive endoderm comprises a basal medium, a TGF-beta superfamily member, an FGF superfamily member, and a Wnt activator (Eg. Rezania et al., 2011).

In yet another embodiment, the culture medium used to differentiate the human pluripotent stem cells to definitive endoderm comprises a basal medium and a TGF-beta superfamily member (Eg. D'Amour et al., 2005).

In another embodiment, the culture medium used to differentiate the human pluripotent stem cells to definitive endoderm comprises a basal medium and a molecule that promotes SMAD phosphorylation.

In yet another embodiment, the endodermal progenitor cells are further differentiated to form pancreatic cells, respiratory cells, intestinal cells or liver cells.

In one embodiment, the endoderm cells derived from the pluripotent stem cells express one or more of CXCR4, SOX17, GATA-4, FOXA2, AFP, CER1, C-KIT, EPCAM, SNAI1, GSC, E-Cad, or N-Cad.

Also provided herein are culture media compositions and kits useful for inducing germ layer differentiation and screening assays for agents that can modulate the differentiation of the cells or for primary or secondary screens of the cells generated by the methods described herein.

Accordingly, in one embodiment, the present disclosure provides a kit comprising a medium with an osmolality of between 260 and 310 mOsm/Kg and a concentrated supplement. In an embodiment, the concentrated supplement comprises a physiological diluent and a salt, such as sodium chloride. In another embodiment, the concentrated supplement further comprises a protein. In one embodiment, the protein is albumin, optionally derived from an animal source or a recombinant source, such as human recombinant albumin.

The medium can be any cell culture medium. In one embodiment, the medium is a pluripotent stem cell maintenance medium. In another embodiment, the medium comprises a mixture of DMEM and F-12 and optionally further comprises vitamins, salts, trace elements, selenium, insulin, lipids, proteins, amino acids, TGF-beta, FGF2, or mixtures thereof. In another embodiment, the medium is TeSR™-E8™, Essential 8™, or E8.

In yet another embodiment, the cells are cultured in neuronal cell differentiation medium comprising DMEM-F12, N2, B27 or combinations thereof, non-essential amino acids, hormones, lipids, BDNF, GDNF, ascorbic acid, retinoic acid, TGF3 (neurons), sonic hedgehog (SHH), thyroid hormone, any member of the BMP family, EGF and PDGF (oligodendrocytes), cyclopamine or any other SHH inhibitor (astrocytes) to produce differentiated cells. The differentiated cells are optionally propagated and maintained on a coated culture dish as described herein. For differentiation, bFGF is removed. In one embodiment, the differentiated cells comprise neurons, astrocytes or oligodendrocytes, which are optionally identified based on the presence of markers selected from TUJ1, MAP2 (neurons), A2B5, GFAP, GLAST (glial cells, astrocytes and radial glial cells), FGFR1, FGFR2, FGFR3, FGFR4, O4, OLIG2, GalC, and NG2 (oligodendrocytes).

### Endodermal/Mesodermal Differentiation

Endodermal/mesodermal germ layer can be induced by culturing stem ells in a culture media with an osmolality range higher than 280 mOsm/kg, optionally 290-340 mOsm/kg for dissociated cells cultured in the microwell device and/or in cell suspension prior to plating on coated culture dishes by the methods described herein and an osmolality above 320 mOsm/kg, optionally 320 to 340 mOsm/kg, for dissociated cells plated directly onto coated culture dishes by the methods described herein.

The osmolality of the culture media used in the methods described herein is higher than 280 mOsm/kg for inducing or enriching endodermal/mesodermal progenitor cells. In another embodiment, the osmolality of the culture medium is 290 to 340 mOsm/kg, for inducing or enriching endodermal/mesodermal progenitor cells. In yet another embodiment, the osmolality of the culture media used in the methods described herein is higher than 320 mOsm/kg for inducing or enriching endodermal/mesodermal progenitor cells. In yet a further embodiment, the osmolality of the culture medium is 320 to 340 mOsm/kg for inducing or enriching endodermal/mesodermal progenitor cells.

A culture medium at an osmolality higher than 280 mOsm/kg, optionally higher than 320 mOsm/kg, provides differentiation into mesodermal fate, which can give rise to mesenchymal stem cells, chondrocytes, cardiomyocytes, hematopoietic stem cells and skeletal cells. In another embodiment, a culture medium at an osmolality higher than 280 mOsm/kg, optionally higher than 320 mOsm/kg, provides differentiation into endodermal fate, which can give rise to pancreas, intestinal cells and liver cells.

The following example is intended to further illustrate certain particularly preferred embodiments of the invention and is not intended to limit the scope of the invention in any way.

### Various Notes & Examples

Example 1 can include subject matter such as can include a method of hyperthermic treatment of body fluid of a human patient comprising: diverting at least a portion of a body fluid through a body fluid inlet from a body; hyperthermically treating the body fluid with a hyperthermic treatment assembly including: measuring a viral load of a pathogen in the body fluid, determining a target treatment temperature based on the measured viral load, and heating the body fluid to the target treatment temperature to decrease the viral load in the body fluid; returning the body fluid after hyperthermic treating to the body through a body fluid outlet; and repeating hyperthermically treating the body fluid with another portion of body fluid diverted through the hyperthermic treatment assembly.

Example 2 can include, or can optionally be combined with the subject matter of Example 1, to optionally include wherein repeating hyperthermically treating the body fluid includes repeating hyperthermically treating the body fluid until a target viral load is measured.

Example 3 can include, or can optionally be combined with the subject matter of one or any combination of Examples 1 or 2 to optionally include cooling the body fluid after heating to a return temperature prior to returning the body fluid to the body.

Example 4 can include, or can optionally be combined with the subject matter of one or any combination of Examples 1-3 to optionally include wherein the cooling the body fluid includes active heat exchange between the body fluid and a cooling heat exchanger.

Example 5 can include, or can optionally be combined with the subject matter of one or any combination of Examples 1-4 to optionally include wherein heating the body fluid to the target temperature includes: heating a heating core to a core temperature based on the target treatment temperature, and delivering the body fluid through a body fluid conduit wrapped around the heating core and heating the body fluid to the target treatment temperature with the heating core.

Example 6 can include, or can optionally be combined with the subject matter of Examples 1-5 to optionally include filtering the body fluid after heating the body fluid to the target treatment temperature, filtering including removing dead or dying pathogens and dead or dying pathogen infected cells from the body fluid.

Example 7 can include, or can optionally be combined with the subject matter of Examples 1-6 to optionally include wherein returning the body fluid after hyperthermic treating includes: measuring a flow rate of the body fluid, and controlling the flow rate of the body fluid according to the measured flow rate.

Example 8 can include, or can optionally be combined with the subject matter of Examples 1-7 to optionally include wherein diverting at least the portion of the body fluid includes diverting at least the portion of the body fluid from a first femoral artery, and returning the body fluid after hyperthermic treating includes returning the body fluid to a second femoral artery.

Example 9 can include, or can optionally be combined with the subject matter of Examples 1-8 to optionally include wherein the hyperthermic treatment of body fluid comprises treating one or more chronic infectious diseases.

Example 10 can include, or can optionally be combined with the subject matter of Examples 1-9 to optionally include wherein the hyperthermic treatment of body fluid comprises treating at least one of Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Yellow Fever, Hep A, Hep B, Hep C, the rhino virus, polio, and chickenpox virus and HIV.

Example 11 can include, or can optionally be combined with the subject matter of Examples 1-10 to optionally include administering immunotherapy treatment to the body fluid between diverting and returning of the body fluid.

Example 12 can include, or can optionally be combined with the subject matter of Examples 1-11 to optionally include a method of hyperthermic treatment of body fluid of a human patient comprising: diverting at least a portion of a body fluid through a body fluid inlet from a body; hyperthermically treating the body fluid with a hyperthermic treatment assembly including: determining a target treatment temperature based on a measured viral load of the body fluid, heating the body fluid to the target treatment temperature with a hyperthermic heat exchanger, heating decreases the viral load in the body fluid, cooling the body fluid to near a body temperature after heating of the body fluid; and returning the body fluid after hyperthermic treating to the body through a body fluid outlet.

Example 13 can include, or can optionally be combined with the subject matter of Examples 1-12 to optionally include repeating hyperthermically treating the body fluid with another portion of body fluid diverted through the hyperthermic treatment assembly.

Example 14 can include, or can optionally be combined with the subject matter of Examples 1-13 to optionally include wherein hyperthermically treating the body fluid includes: measuring a first output temperature of the body fluid near an output of the hyperthermic heat exchanger and measuring a second output temperature of the body fluid near an output of a cooling heat exchanger downstream from the hyperthermic heat exchanger, and cooling the body fluid to near body temperature after heating includes cooling according to the measured first and second output temperatures.

Example 15 can include, or can optionally be combined with the subject matter of Examples 1-14 to optionally include wherein hyperthermically treating the body fluid includes: measuring a first input temperature of the body fluid near an input of the hyperthermic heat exchanger and measuring a first output temperature of the body fluid near an output of the hyperthermic heat exchanger, and heating the body fluid to the target treatment temperature includes heating to the target treatment temperature according to the measured first input and first output temperatures.

Example 16 can include, or can optionally be combined with the subject matter of Examples 1-15 to optionally include wherein the cooling the body fluid to near body temperature includes active heat exchange between the body fluid and a cooling heat exchanger.

Example 17 can include, or can optionally be combined with the subject matter of Examples 1-16 to optionally include wherein heating the body fluid to the target temperature includes: heating a heating core to a core temperature based on the target treatment temperature, and delivering the body fluid through a body fluid conduit wrapped around the heating core and heating the body fluid to the target treatment temperature with the heating core.

Example 18 can include, or can optionally be combined with the subject matter of Examples 1-17 to optionally include filtering the body fluid after heating the body fluid to the target treatment temperature, filter including removing dead or dying pathogens and dead or dying pathogen infected cells from the body fluid.

Example 19 can include, or can optionally be combined with the subject matter of Examples 1-18 to optionally include wherein returning the body fluid after hyperthermic treating includes: measuring a flow rate of the body fluid, and controlling the flow rate of the body fluid according the measured flow rate.

Example 20 can include, or can optionally be combined with the subject matter of Examples 1-19 to optionally include wherein the hyperthermic treatment of body fluid comprises treating one or more chronic infectious diseases.

Example 21 can include, or can optionally be combined with the subject matter of Examples 1-20 to optionally include wherein the hyperthermic treatment of body fluid comprises treating at least one of Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Yellow Fever, Hep A, Hep B, Hep C, the rhino virus, polio, and chickenpox virus and HIV.

Example 22 can include, or can optionally be combined with the subject matter of Examples 1-21 to optionally include a hyperthermic treatment system for use with body fluids of a human patient comprising: a body fluid inlet; a body fluid outlet; a body fluid pump in communication with the body fluid inlet and outlet, the body fluid pump moves body fluid through the hyperthermic treatment system; a hyperthermic treatment assembly in communication with the body fluid inlet and outlet, the hyperthermic treatment circuit includes: a hyperthermic heat exchanger interposed between the body fluid inlet and outlet, the hyperthermic heat exchanger heats the body fluid to a target treatment temperature, a cooling heat exchanger interposed between the hyperthermic heat exchanger and the body fluid outlet, the cooling heat exchanger cools the body fluid after heating by the hyperthermic heat exchanger, and a body fluid controller coupled with at least the hyperthermic heat exchanger, the body fluid controller controls the hyperthermic heat exchanger to heat the body fluid to the target treatment temperature.

Example 23 can include, or can optionally be combined with the subject matter of Examples 1-22 to optionally include wherein the body fluid controller is coupled with the cooling heat exchanger, and the body fluid controller controls the cooling heat exchanger to cool the body fluid to near a body temperature.

Example 24 can include, or can optionally be combined with the subject matter of Examples 1-23 to optionally include wherein the controller includes a viral load temperature module, the viral load temperature module includes a database of a plurality of viral load values and a plurality of target treatment temperatures, each of the viral load values is associated with a corresponding target treatment temperature of the plurality of target treatment temperatures.

Example 25 can include, or can optionally be combined with the subject matter of Examples 1-24 to optionally include wherein the hyperthermic treatment assembly includes: a first temperature sensor upstream from the hyperthermic heat exchanger, a second temperature sensor downstream from the hyperthermic heat exchanger, and wherein each of the first and second temperature sensors is coupled with the body fluid controller.

Example 26 can include, or can optionally be combined with the subject matter of Examples 1-25 to optionally include wherein the second temperature sensor is upstream from the cooling heat exchanger, and the hyperthermic treatment assembly includes a third temperature sensor downstream from the cooling heat exchanger, and the third temperature sensor is coupled with the body fluid controller.

Example 27 can include, or can optionally be combined with the subject matter of Examples 1-26 to optionally include a perfusate filter downstream from the hyperthermic heat exchanger, the perfusate filter includes: a plurality of perforated flow channels, and a waste removal reservoir around the plurality of perforated flow channels, the plurality of perforated flow channels are in communication with the waste removal reservoir through perforations in the perforated flow channels.

Example 28 can include, or can optionally be combined with the subject matter of Examples 1-27 to optionally include wherein the hyperthermic heat exchanger includes: a heating core, and a body fluid conduit in communication with the body fluid inlet and outlet, wherein the body fluid conduit wraps around the heating core.

Example 29 can include, or can optionally be combined with the subject matter of Examples 1-28 to optionally include wherein the cooling heat exchanger includes: a cooling coil extending within a cooling jacket, and a body fluid conduit extending through the cooling coil and within the cooling jacket.

Example 30 can include, or can optionally be combined with the subject matter of Examples 1-29 to optionally include a flow sensor downstream from at least the hyperthermic heat exchanger, a flow controller adjacent to the flow sensor, and wherein the flow sensor and the flow controller are coupled with the body fluid controller, and the body fluid controller adjusts a flow controller output according to measurements of a body fluid flow from the flow sensor.

Example 31 can include, or can optionally be combined with the subject matter of Examples 1-30 to optionally include an immunogenic composition comprising in vitro matured dendritic cells.

Example 32 can include, or can optionally be combined with the subject matter of Examples 1-31 to optionally include wherein the dendritic cells are matured by contact with an immunogen from a virus.

Example 33 can include, or can optionally be combined with the subject matter of Examples 1-32 to optionally include wherein the immunogen is a viral RNA.

Example 34 can include, or can optionally be combined with the subject matter of Examples 1-33 to optionally include wherein the virus is Marburg virus, Ebola, Hantavirus, H5N1 strain of bird flu, Lassa virus, Junin virus, Dengue fever, Crimea-Congo fever virus, Bolivian hemorrhagic fever, HIV, Yellow Fever, Hep A, Hep B, Hep C, the rhino virus, polio, and chickenpox virus or Kyasanur Forest Virus (KFD).

Example 35 can include, or can optionally be combined with the subject matter of Examples 1-34 to optionally include a pharmaceutically acceptable carrier.

Example 36 can include, or can optionally be combined with the subject matter of Examples 1-35 to optionally include a method to treat a viral infection comprising administering an effective amount of the immunogenic composition of any one of the examples to a subject in need thereof so as to treat said viral infection.

Example 37 can include, or can optionally be combined with the subject matter of Examples 1-36 to optionally include wherein the dendritic cells are autologous to said subject.

Example 38 can include, or can optionally be combined with the subject matter of Examples 1-37 to optionally include hyperthermia treatment.

Example 39 can include, or can optionally be combined with the subject matter of Examples 1-38 to optionally include a method to produce in vitro matured dendritic cells comprising isolating dendritic cells from a subject, expanding said dendritic cells in vitro and contacting said cells with RNA from virus that has infected said subject so as to produce mature dendritic cells.

Example 40 can include, or can optionally be combined with the subject matter of Examples 1-39 to optionally include wherein the virus is Marburg virus, Ebola, Hantavirus, H5N1 strain of bird flu, Lassa virus, Junin virus, Dengue fever, Crimea-Congo fever virus, Bolivian hemorrhagic fever, HIV, Yellow Fever, Hep A, Hep B, Hep C, the rhino virus, polio, and chickenpox virus or Kyasanur Forest Virus (KFD).

### Example 41

Ebola Virus-Vaccine Protects Nonhuman subjects against Lethal Ebola Virus Challenge

### Introduction

Countermeasure development will ultimately require an improved understanding of protective immune correlates and how they are modulated during infection. This proves difficult when infected individuals who succumb to filoviral disease fail to mount an early immune response. These fast-moving hemorrhagic fever diseases result in immune dysregulation, as demonstrated by the lack of a virus-specific Ab response and a great reduction in gross T-cell numbers, leading to uncontrolled viral replication and multi-organ infection and failure. Conversely, survivors of Ebola virus (EBOV) disease exhibit an early and transient IgM response, which is quickly followed by increasing levels of virus-specific IgG and cytotoxic T lymphocytes (CTL). These observations suggest that humoral and cell-mediated immune responses play a role in conferring protection against disease. These data are also supported by numerous preclinical efficacy studies demonstrating the contribution of vaccine-induced adaptive immunity to the protection against lethal challenge. However, mounting evidence has demonstrated a critical role for T cells in providing protection where efficacy was greatly associated with the functional phenotype of CD8⁺ T cells. Although these recent studies highlight the importance of T cells in providing protection, their precise contributions remain uncharacterized and controversial. Furthermore, little detailed analysis of this response driven by a protective vaccine has been reported.

To help expand upon these data, a novel polyvalent-filovirus vaccine (an immunogenic composition) was developed comprised by three DNA plasmids encoding the envelope glycoprotein (GP) genes of Marburg Marburgvirus (MARV), Sudan ebolavirus (SUDV), or Zaire ebolavirus (ZEBOV), adopting the multiagent approach, and determined its capacity for inducing protective efficacy and broad CTL in rodent preclinical studies. In addition, T-cell responses were extensively analyzed including the use of a novel method for epitope identification and characterization described herein. As a filoviral vaccine candidate, an “enhanced” DNA (E-DNA)-based platform exhibits many advantages given recent advances in genetic optimization and delivery techniques. As such, each GP was genetically optimized, subcloned into modified mammalian expression vectors, and then delivered using in vivo electroporation. Vaccination in preclinical rodent studies induced robust neutralizing Abs (NAbs) and CTL expressing Th1-type markers, and was completely protective against challenge with MARV and ZEBOV. Furthermore, vaccine-induced T-cell responses exhibited great epitopic breadth as extensively analyzed using a novel modified assay described herein. In total, 52 novel T-cell epitopes from two different mouse genetic backgrounds were identified (19 of 20 MARV epitopes, 15 of 16 SUDV, and 18 of 22 ZEBOV) and occurred primarily in highly conserved regions of their respective GPs. These data represent the most comprehensive report of preclinical GP epitopes to date, and provides a tool by which T-cell responses may be further evaluated in comparative studies and in relation to protective efficacy in the preclinic, and later in nonhuman primate studies.

### Materials and Methods

GenBank protein IDs: Identification of proteins in are as follows: MARV Durba (05DRC99) '99: ABE27085; Uganda (01Uga07) '07: ACT79229; Durba (07DRC99) '99: ABE27078; Ozolin '75: VGP_MABVO; Musoke '80: VGP_MABVM; Popp '67: VGP_MABVP; Leiden '08: AEW 11937; Angola '05: VGP_MABVA; Ravn '87: VGP_MABVR; Durba (09DRC99) '99; ABE27092; Uganda (02Uga07) '07: ACT79201. SUDV: Boniface '76: VGP_EBOSB; Maleo '79: VGP_EBOSM; Yambio '04: ABY75325; Gulu '00: VGP_EBOSU. ZEBOV: Booue '96: AAL25818; Mayibout '96: AEK25495; Mekouka '94: AAC57989, VGP_EBOG4; Kikwit '95: VGP_EBOZ5; Yambuku (Ekron) '76: VGP_EBOEC; Yambuku (Mayinga) '76: VGP_EBOZM; Kasai '08: AER59712; Kassai '07: AER59718; Etoumbi '05: ABW34742; Mbomo/Mbandza '03: ABW34743.

### Filoviral Vaccine GP Immunogen Sequences

Zaire Ebolavirus Consensus (ZEBOV CON VACCINE; pEBOZ):

(SEQ ID NO: 5)
MGVTGILQLPRDRFKRTSFFLWVIILFQRTFSIPLGVIHNSTLQV
SDVDKLVCRDKLSSTNQLRSVGLNLEGNGVATDVPSATKRWGFRS
GVPPKVVNYEAGEWAENCYNLEIKKPDGSECLPAAPDGIRGFPRC
RYVHKVSGTGPCAGDFAFHKEGAFFLYDRLASTVIYRGTTFAEGV
VAFLILPQAKKDFFSSHPLREPVNATEDPSSGYYSTTIRYQATGF
GTNETEYLFEVDNLTYVQLESRFTPQFLLQLNETIYTSGKRSNTT
GKLIWKVNPEIDTTIGEWAFWETKKNLTRKIRSEELSFTAVSNRA
KNISGQSPARTSSDPGTNTTTEDHKIMASENSSAMVQVHSQGREA
AVSHLTTLATISTSPQSPTTKPGPDNSTHNTPVYKLDISEATQVE
QHHRRTDNDSTASDTPPATTAAGPPKAENTNTSKSTDLLDPATTT
SPQNHSETAGNNNTHHQDTGEESASSGKLGLITNTIAGVAGLITG
GRRTRREAIVNAQPKCNPNLHYWTTQDEGAAIGLAWIPYFGPAAE
GIYTEGLMHNQDGLICGLRQLANETTQALQLFLRATTELRTFSIL
NRKAIDFLLQRWGGTCHILGPDCCIEPHDWTKNITDKIDQIIHDF
VDKTLPDQGDNDNWWTGWRQWIPAGIGVTGVIIAVIALFCICKFV
F

Sudan Ebolavirus Consensus (SUDV CON VACCINE; pEBOS):

(SEQ ID NO: 6)
MEGLSLLQLPRDKERKSSFEVWVIILFQKAFSMPLGVVTNSTLEV
TEIDQLVCKDHLASTDQLKSVGLNLEGSGVSTDIPSATKRWGFRS
GVPPKVVSYEAGEWAENCYNLEIKKPDGSECLPPPPDGVRGFPRC
RYVHKAQGTGPCPGDYAFHKDGAFFLYDRLASTVIYRGVNFAEGV
IAFLILAKPKETFLQSPPIREAVNYTENTSSYYATSYLEYEIENF
GAQHSTTLFKINNNTFVLLDRPHTPQFLFQLNDTIHLHQQLSNTT
GKLIWTLDANINADIGEWAFWENKKNLSEQLRGEELSFETLSLNE
TEDDDATSSRTTKGRISDRATRKYSDLVPKDSPGMVSLHVPEGET
TLPSQNSTEGRRVDVNTQETITETTATIIGTNGNNMQISTIGTGL
SSSQILSSSPTMAPSPETQTSTTYTPKLPVMTTEEPTTPPRNSPG
STTEAPTLTTPENITTAVKTVLPQESTSNGLITSTVTGILGSLGL
RKRSRRQVNTRATGKCNPNLHYWTAQEQHNAAGIAWIPYFGPGAE
GIYTEGLMHNQNALVCGLRQLANETTQALQLFLRATTELRTYTIL
NRKAIDFLLRRWGGTCRILGPDCCIEPHDWTKNITDKINQIIHDF
IDNPLPNQDNDDNWWTGWRQWIPAGIGITGIIIAIIALLCVCKLL
C

Marburg Marburgvirus Angola (MARV VACCINE; pMARV):

(SEQ ID NO: 7)
MKTTCLLISLILIQGVKTLPILEIASNIQPQNVDSVCSGTLQKTE
DVHLMGFTLSGQKVADSPLEASKRWAFRAGVPPKNVEYTEGEEAK
TCYNISVTDPSGKSLLLDPPTNIRDYPKCKTIHHIQGQNPHAQGI
ALHLWGAFFLYDRIASTTMYRGKVFTEGNIAAMIVNKTVHKMIFS
RQGQGYRHMNLTSTNKYWTSSNGTQTNDTGCFGTLQEYNSTKNQT
CAPSKKPLPLPTAHPEVKLTSTSTDATKLNTTDPNSDDEDLTTSG
SGSGEQEPYTTSDAATKQGLSSTMPPTPSPQPSTPQQGGNNTNHS
QGVVTEPGKTNTTAQPSMPPHNTTTISTNNTSKHNLSTPSVPIQN
ATNYNTQSTAPENEQTSAPSKTTLLPTENPTTAKSTNSTKSPTTT
VPNTTNKYSTSPSPTPNSTAQHLVYFRRKRNILWREGDMFPFLDG
LINAPIDFDPVPNTKTIFDESSSSGASAEEDQHASPNISLTLSYF
PKVNENTAHSGENENDCDAELRIWSVQEDDLAAGLSWIPFFGPGI
EGLYTAGLIKNQNNLVCRLRRLANQTAKSLELLLRVTTEERTFSL
INRHAIDFLLARWGGTCKVLGPDCCIGIEDLSRNISEQIDQIKKD
EQKEGTGWGLGGKWWTSDWGVLTNLGILLLLSIAVLIALSCICRI
FTKYIG

### GP Immunogen Sequences for MARV Expansion Vaccines

### Marburg Marburgvirus—Ravn Cluster Consensus (MARV-RAVV CON VACCINE); (Ravn, Durba (09DRC99) and Uganda (02Uga07)):

(SEQ ID NO: 8)
MKTIYFLISLILIQSIKTLPVLEIASNSQPQDVDSVCSGTLQKTE
DVHLMGFTLSGQKVADSPLEASKRWAFRTGVPPKNVEYTEGEEAK
TCYNISVTDPSGKSLLLDPPSNIRDYPKCKTVHHIQGQNPHAQGI
ALHLWGAFFLYDRVASTTMYRGKVFTEGNIAAMIVNKTVHRMIFS
RQGQGYRHMNLTSTNKYWTSSNETRRNDTGCFGILQEYNSTNNQT
CSPSLKPPSLPTVTPSIHSTNTQINTAKSGTMNPSSDDEDLMISG
SGSGEQGPHTTLNVVTEQKQSSTILSTPSLHPSTSQHEQNSTNPS
RHAVTEHNGTDPTTQPATLLNNTNTTPTYNTLKYNLSTPSPPTRN
ITNNDTQRELAESEQTNAQLNTTLDPTENPTTAQDTNSTTNIIMT
TSDITSKHPTNSSPDSSPTTRPPIYFRKKRSIFWKEGDIFPFLDG
LINTEIDFDPIPNTETIFDESPSFNTSTNEEQHTPPNISLTFSYF
PDKNGDTAYSGENENDCDAELRIWSVQEDDLAAGLSWIPFFGPGI
EGLYTAGLIKNQNNLVCRLRRLANQTAKSLELLLRVTTEERTFSL
INRHAIDFLLTRWGGTCKVLGPDCCIGIEDLSKNISEQIDKIRKD
EQKEETGWGLGGKWWTSDWGVLTNLGILLLLSIAVLIALSCICRI
FTKYIG

### Marburg Marburgvirus—Ozolin Cluster Consensus (MARV-OZO CON VACCINE); (Ozolin, Uganda (01Uga07), and Durba (05 and 07DRC99)):

(SEQ ID NO: 9)
MRTTCFFISLILIQGIKTLPILEIASNDQPQNVDSVCSGTLQKTE
DVHLMGFTLSGQKVADSPLEASKRWAFRTGVPPKNVEYTEGEEAK
TCYNISVTDPSGKSLLLDPPTNVRDYPKCKTIHHIQGQNPHAQGI
ALHLWGAFFLYDRIASTTMYRGKVETEGNIAAMIVNKTVHKMIFS
RQGQGYRHMNLTSTNKYWTSSNGTQTNDTGCFGTLQEYNSTKNQT
CAPSKTPPPPPTARPEIKPTSTPTDATRLNTTNPNSDDEDLTTSG
SGSGEQEPYTTSDAVTKQGLSSTMPPTPSPQPGTPQQGGNNTNHS
QDAATELDNTNTTAQPPTPSHNTTTISTNNTSKHNLSTLSEPPQN
TTNPNTQSMATENEKTSAPPKTTLPPTESPTTEKSTNNTKSPTTM
EPNTTNGHETSPSSTPNSTTQHLIYFRRKRSILWREGDMFPFLDG
LINAPIDFDPVPNTKTIFDESSSSGASAEEDQHASSNISLTLSYL
PHTSENTAYSGENENDCDAELRIWSVQEDDLAAGLSWIPFEGPGI
EGLYTAGLIKNQNNLVCRLRRLANQTAKSLELLLRVTTEERTFSL
INRHAIDELLTRWGGTCKVLGPDCCIGIEDLSRNISEQIDQIKKD
EQKEGTGWGLGGKWWTSDWGVLTNLGILLLLSIAVLIALSCICRI
FTKYIG

### Marburg Marburgvirus—Musoke Cluster Consensus (MARV-MUS CON VACCINE); (Musoke, Popp, and Leiden):

(SEQ ID NO: 10)
MKTTCLFISLILIQGIKTLPILEIASNNQPQNVDSVCSGTLQKTE
DVHLMGFTLSGQKVADSPLEASKRWAFRTGVPPKNVEYTEGEEAK
TCYNISVTDPSGKSLLLDPPTNIRDYPKCKTIHHIQGQNPHAQGI
ALHLWGAFFLYDRIASTTMYRGRVFTEGNIAAMIVNKTVHKMIFS
RQGQGYRHMNLTSTNKYWTSNNGTQTNDTGCFGALQEYNSTKNQT
CAPSKIPSPLPTARPEIKPTSTPTDATKLNTTDPNSDDEDLATSG
SGSGEQEPHTTSDAVTKQGLSSTMPPTPSPQPSTPQQEGNNTDHS
QDAVTEPNKTNTTAQPSMPPHNTTAISTNNTSKHNFSTLSAPLQN
TTNYDTQSTATENEQTSAPSKTTLPPTGNLTTAKSTNNTKGPTTT
APNMTNGHLTSPSPTPNPTTQHLVYFRKKRSILWREGDMFPFLDG
LINAPIDFDPVPNTKTIFDESSSSGASAEEDQHASPNISLTLSYF
PNINENTAYSGENENDCDAELRIWSVQEDDLAAGLSWIPFFGPGI
EGLYTAGLIKNQNNLVCRLRRLANQTAKSLELLLRVTTEERTFSL
INRHAIDFLLTRWGGTCKVLGPDCCIGIEDLSRNISEQIDQIKKD
EQKEGTGWGLGGKWWTSDWGVLTNLGILLLLSIAVLIALSCICRI
FTKYIG

### Transfections and Immunoblotting

Human Kidney (HEK) 293T cells were cultured, transfected, and harvested by methods available to the art. Briefly, cells were grown in DMEM with 10% fetal bovine serum (FBS), 1% Pen-strep, sodium pyruvate, and L-glutamine. Cells were cultured in 150 mm Corning dishes and grown to 70% confluence overnight in a 37° incubator with 5% CO₂. Dishes were transfected with 10-25 μg of Filoviridae pDNA using either a Calphos Mammalian Transfection Kit protocol (Clonetech) or Lipofectamine 2000 reagent (Invitrogen) per the manufacturer's protocol and then incubated for 24-48 hours. Cells were harvested with ice cold phosphate-buffered saline (PBS), centrifuged and washed, and then pelleted for Western immunoblot or FACS analysis. Standard western blotting was used and GP-specific MAbs for GP1 detection were generated as described

### Animals, Vaccinations, and Challenge

Adult female C57BL/6 (H-2b), BALB/cJ (H-2d), and B10.Br (H-2k) mice were purchased from The Jackson Laboratory (Bar Harbor, Me.), whereas Hartley guinea pigs were from Charles River (Wilmington, Mass.).

Mice were immunized i.m. by needle injection with 40 μg of plasmid resuspended in water, whereas guinea pigs were immunized i.d., with 200 μg of each into three separate vaccination sites. Vaccinations were immediately followed by electroporation at the same site as previously described. Briefly, a three-pronged CELLECTRA adaptive constant current Minimally Invasive Device was inserted ˜2 mm i.d. (Inovio Pharmaceuticals, Blue Bell, Pa.). Square-wave pulses were delivered through a triangular 3-electrode array consisting of 26-gauge solid stainless steel electrodes and two constant current pulses of 0.1 Amps were delivered for 52 microsecond/pulse separated by a 1 second delay.

For lethal challenge studies, challenges were limited to rodent-adapted ZEBOV and MARV, as SUDV adapted for lethality in rodents are not yet available. Guinea pigs were challenged 28 days after the final vaccination by i.p. injection with 1,000 LD50 of guinea pig-adapted ZEBOV (21.3 FFU/animal) or 1,000 LD50MARV-Angola (681 TCID50/animal), which was made in-house. Briefly, the guinea pig-adapted MARV was made by the serial passage of wild-type MARV-Angola in outbred adult female Hartley guinea pigs. Seven days after inoculation, the animals were euthanized and livers were harvested and homogenized. This homogenate was then injected i.p. into naïve adult guinea pigs and the process repeated until animals lost weight, gloss of hair, and succumbed to infection similar to EBOV adaptation in guinea pigs. For mouse lethal challenge studies, mice were injected i.p. with 200 μl of a 1,000 LD50 (10 FFU/animal) of mouse-adapted ZEBOV. All animals were weighed daily and monitored for disease progression using an approved score sheet for at least 18 days for mice and 22 days for guinea pigs.

### ELISA and Neutralization Assays.

Ab titers were determined using 96-well ELISA plates coated with either sucrose-purified MARV-Ozolin GP or ZGP (SGP was not available for this study), or with negative control sucrose-purified Nipah G protein at a concentration of 1:2,000, as previously described. Briefly, the plates were then incubated for 18 hours at 4° C., washed with PBS and 0.1% Tween-20, and 100 μl/sample of the sera were tested in triplicate (at dilutions 1:100, 1:400, 1:1,600, and 1:6,400 in PBS with 5% skim milk and 0.5% Tween-20). Following an incubation at 37° C. for 1 hour in a moist container, the plates were washed and then 100 μl of goat antimouse IgG-conjugated HRP antibody (Cedarlane, Burlington, N.C.) was added (1:2,000 dilution) and incubated for another 37° C. for 1 hour in a moist container. After a wash, 100 μl of the ABST (2,2′-azino-bis(3-ethylbenzothiazoline-6-sulphonic acid) and peroxidase substrate (Cedarlane) was added to visualize Ab binding. Again in a moist container, the plate was incubated for 30 minutes at 37° C. and then later read at 405 nm. Positive binding results were characterized by being >3 SD when subtracting the positive control from the negative control serum.

The ZEBOV neutralization assay was performed as previously described. Briefly, Sera collected from immunized mice and guinea pigs were inactivated at 56° C. for 45 minutes and serial dilutions of each sample (1:20, 1:40, etc., for mice and 1:50 for guinea pigs, in 50 μl of DMEM) was mixed with equal volume of ZEBOV expressing the enhanced green fluorescent protein (EGFP) reporter gene (ZEBOV-EGFP) (100 transducing units/well, according to EGFP expression) and incubated at 37° C. for 90 minutes. The mixture was then transferred onto subconfluent VeroE6 cells in 96-well flat-bottomed plates and incubated for 5-10 minutes at room temperature. Control wells were infected with equal amounts of the ZEBOV-EGFP virus without addition of serum or with non-immune serum. DMEM of 100 μl supplemented with 20% FBS was then added to each well, and plates were incubated at 37° C. in 5% CO2 for 48 hours. Alternatively, neutralization of MARV-Angola 368 was assessed using an immunofluorescent assay. A primary rabbit anti-MARV Ab and secondary goat anti-rabbit IgG FITC-conjugated Ab was used for detection. NAbs against SUDV-Boniface were assayed based on cytopathic effect on CV-1 cells. Cells were incubated with equal parts of immunized sera and SUDV-Boniface for 10 days before subsequently fixed with 10% buffered formalin for 24 hours and examined under a light microscope. EGFP and FITC positive cells were counted in each well and sample dilutions showing >50% reduction in the number of green cells compared with controls scored positive for NAb. Alternatively, NAbs against SUDV-Boniface were assayed based on cytopathic effect on CV-1 cells.

### Splenocyte Isolation

Spleens were harvested 8-11 days following the final immunization as previously described. Briefly, spleens were placed in RPMI 1640 medium (Mediatech, Manassas, Va.) supplemented with 10% FBS, 1× Antibiotic-Antimycotic (Invitrogen), and 1×β-ME (Invitrogen). Splenocytes were isolated by mechanical disruption of the spleen using a Stomacher machine (Seward Laboratory Systems, Bohemia, N.Y.), and the resulting product was filtered using a 40 μm cell strainer (BD Biosciences, San Jose, Calif.). The cells were then treated for 5 minutes with ACK lysis buffer (Lonza, Switzerland) for lysis of RBCs, washed in PBS, and then resuspended in RPMI medium for use in ELISPOT or FACS assay.

### ELISPOT Assays

Standard IFNγ ELISPOT assay has been described. Briefly, 96-well plates (Millipore, Billerica, Mass.) were coated with antimouse IFN capture antibody and incubated for 24 hours at 4° C. (R&D Systems, Minneapolis, Minn.). The following day, plates were washed with PBS and then blocked for 2 hours with blocking buffer (1% BSA and 5% sucrose in PBS). Splenocytes (1-2×105 cells/well) were plated in triplicate and stimulated overnight at 37° C. in 5% CO2 and in the presence of either RPMI 1640 (negative control), Con A (positive control), or GP peptides either individually (15-mers overlapping by 9 amino acids and spanning the lengths of their respective GP) or whole pooled (2.5 g/ml final). After 18-24 hours of stimulation, the plates were washed in PBS and then incubated for 24 hours at 4° C. with biotinylated antimouse IFNmAb (R&D Systems). Next, the plates were washed again in PBS, and streptavidin-alkaline phosphatase (MabTech, Nacka Strand, Sweden) was added to each well and incubated for 2 hours at room temperature. Lastly, the plates were washed again in PBS and then BCIP/NBT Plus substrate (MabTech) was added to each well for 5-30 minutes for spot development. As soon as the development process was complete upon visual inspection, the plate was rinsed with distilled water and then dried overnight at room temperature. Spots were enumerated using an automated ELISPOT reader (Cellular Technology, Shaker Heights, Ohio).

For comprehensive analysis of T-cell breadth, standard IFNγ ELISPOT was modified herein as previously described. Identification and measurement of subdominant and immunodominant T-cell epitopes were assessed by stimulating splenocytes with individual peptides as opposed to whole or matrix peptide pools; the traditional practice of pooling peptides for the sake of sample preservation, such as the use of matrix array pools, results in a reduction of assay sensitivity, because total functional responses in pools containing multiple epitope-displaying peptides will effectively lower assay resolution, i.e., “drown-out” those of lower magnitude. Thus, modified ELISPOT was performed with individual peptides (15-mers overlapping by 9 amino acids; 2.5 g/ml final) spanning each GP immunogen. Peptides containing T-cell epitopes were identified (≧10 average IFN γ+spots and ≧80% animal response rate; summarized in and then later confirmed functionally and phenotypically by FACS. No shared or partial epitopes were identified (data not shown), nor did FACS data or web-based epitope prediction software (suggest the presence of a CD4+ or CD8⁺ T-cell epitope that was preserved within consecutive peptides. Here, possible shared/partial T-cell epitopes were addressed for all instances of contiguous peptide responses as identified by modified ELISPOT assay. Cells were stimulated individually with each of the contiguous peptides, as well as paired in combination for direct comparison, and were defined as “shared/partial” if the combined response was not greater than either of the two individual responses. Also, it must be noted, that the epitopic response presented herein may not have been completely comprehensive, because the “15-mer overlapping by 9 amino acids” algorithm for generating peptides is biased towards complete coverage of CD8 T-cell epitopes which may underestimate CD4 T-cell responses due to the nature of class II-restricted epitopes being longer than 15 amino acids. Lastly, amino acid similarity plots were generated using Vector NTI software

### Flow Cytometry

Splenocytes were added to a 96-well plate (1×10⁶ cells/well) and stimulated for 5-6 hours with either individual peptides or “Minimal Peptide Pools” (2.5 μg/ml final). Individual peptides stimulation was used for functional confirmation of all peptides identified by modified ELISPOT as well as phenotypic characterization. Splenocytes and transfected 293 Ts were first prestained with LIVE/DEAD Fixable Violet Dead Cell Stain Kit (Invitrogen). For splenocytes, cells were surface-stained for CD19 (V450; clone 1D3), CD4 (PE-Cy7; clone RM4-5), CD8 (APC-Cy7; clone 53-6.7), and CD44 (PE-Cy5; clone IM7) (BD Biosciences), washed three times in PBS+1% FBS, permeabilized with BD Cytofix/Cytoperm kit, and then stained intracellularly with IFNγ (APC; clone XMG1.2), TNF (FITC; clone MP6-XT22), CD3 (PE-cy5.5; clone 145-2C11), and T-bet (PE; clone 4B10) (eBioscience). GP expression in transfected 293T cells was assessed 24 hours after transfection. Indirect staining was performed following a 30 minutes incubation at 4° C. in PBS+1% FBS containing the indicated mouse-derived GP-specific polyclonal serum reagent (1:200 dilution), each produced by pooling serum from H-2b mice immunized three times with their respective E-DNA vaccine or pVAX1 empty vector control. Cells were then stained with FITC-conjugated goat antimouse IgG (BioLegend, San Diego, Calif.), washed extensively, and then stained for MHC class I (HLA-ABC; PE-Cy7; clone G46-2.6; BD Biosciences). All cells were fixed in 1% paraformaldehyde. All data were collected using a LSRII flow cytometer (BD Biosciences) and analyzed using FlowJo software (Tree Star, Ashland, Oreg.). Splenocytes were gated for activated IFNγ-producing T cells that were CD3+ CD44+, CD4+, or CD8+, and negative for the B cell marker CD19 and viability dye. Statistical analysis. Significance for unrooted phylogenetic trees was determined by maximum-likelihood method and verified by bootstrap analysis and significant support values (≧80%; 1,000 bootstrap replicates) were determined by MEGA version 5 software. Group analyses were completed by matched, two-tailed, unpaired t-test and survival curves were analyzed by log-rank (Mantel-Cox) test. All values are mean±SEM and statistical analyses were performed by GraphPad Prism (La Jolla, Calif.).

### Results

### Vaccine Construction and Expression

Phylogenetic analysis revealed relative conservation among the EBOV GPs (94.4% for SUDV and 92.9% for ZEBOV), whereas the MARV GP (MGP) were more divergent (˜70% conserved). Thus, a consensus strategy, as determined by alignment of the prevailing ZEBOV and SUDV GP amino acid sequences, was adopted for the EBOV GPs, whereas a type-matched strategy was used for MARV using the 2005 Angola outbreak sequence which was solely responsible for the largest and deadliest MARV outbreak. Each GP transgene was genetically optimized, synthesized commercially, and then subcloned into a modified pVAX1 mammalian expression vector. Altogether, a three-plasmid strategy formed the foundation for our novel polyvalent-filovirus vaccine strategy incorporating CD⁺8 T cells.

Described herein is the production in vitro of programmed immune cells comprising: obtaining hematopoetic, mesenchymal, connective tissue, endothelial, cardio, induced pluripotent, osteo, muscle, and/or soft muscle tissue stem cells from a subject; expanding the stem cells from a culture; differentiating the stem cells and; exposing the differentiated stem cells to anthrax particle to generate immune response triggering surface antigen expression in the differentiated cells; and combining said cells so as to comprise a mixture of differentiated cells obtained from about 46% hematopoetic stem cells, about 35% mesenchymal stem cells, about 18% connective tissue, endothelial, cardio, induced pluripotent, osteo, muscle, and/or soft muscle tissue stem cells.

### Discussion

Reported herein is the development and evaluation of a polyvalent-filoviral vaccine in preclinical rodent immunogenicity and efficacy studies. Complete protection against challenge with gpMARV and gpZEBOV was observed following two E-DNA vaccine doses in guinea pigs, as well as with a “single-dose” E-DNA vaccine in mice against mZEBOV To date, genetic vaccination of guinea pigs has included either injection of naked DNA or DNA delivered by gene gun; however, either method required at least three vaccinations to achieve complete protection. Improved protection herein may be due to the induction of robust Abs, because a single E-DNA vaccination generated GP-specific IgG binding titers that were comparable in magnitude with titers in protected animals following gene gun administration; E-DNA vaccination induced 3.85 and 2.18 log 10 ZGP and MGP-specific Ab titers, respectively, after a single administration versus 2.7 and 3.0 after three gene gun vaccinations. For comparison with an alternative “single-dose” protective strategy in guinea pigs, a Ag-coupled virus-like particle platform generated Ab titers that were only slightly higher than observed following E-DNA vaccination. Furthermore, a recombinant adenovirus approach induced ZGP-specific NAb titers that were lower than those from a single E-DNA vaccination (53 reciprocal endpoint dilution titer verses 88 herein). Vaccination with recombinant vesicular stomatitis viruses generated ZGP-specific Ab titers that were similar to the current platform. Altogether, these data demonstrate that E-DNA vaccination was capable of inducing binding and neutralizing Abs that were comparable with non-replicating viral platforms and that these data may help, in part, to explain strong guinea pig survival data herein.

The generation of NAbs by protective E-DNA vaccination may have benefitted by transgene-expressed mature GP structures. In vitro transfection studies confirmed that the vaccine-encoded GP were highly expressed, post-translationally cleaved, transported to the cell surface, and sterically occluded the immunodetection of cell surface molecules. Therefore, it was highly likely that the vaccine immunogens formed herein matured into hetero-trimeric spikes that would otherwise be functional upon virion assembly during infection. This may be important for the generation and display of virologically relevant neutralizing determinants which would be subsequently critical for the induction of conformation-dependent NAbs. Thus, in this regard, the expression of native anchored structures may be superior to soluble derivatives in the capacity for generating NAbs. To better characterize T-cell responses as driven by a protective vaccine, we performed immunogenicity and efficacy studies in mice and determined “single-dose” complete protection against mZEBOV with E-DNA vaccination. The most effective platforms conferring complete protection in this model are virus-like particle, either with or without adjuvant, recombinant adenovirus vaccination, or rRABV vaccination. However, characterization of T-cell responses were severely limited in these studies and were restricted to splenocyte stimulation with either two or one peptides previously described to contain ZGP T-cell epitopes. Herein, induction of robust and broad CTL by protective vaccination is reported as extensively analyzed by a novel modified T-cell assay. In total, 52 novel T-cell epitopes were identified including numerous immunodominant epitopes occurring primarily in highly conserved regions of GP. Of the 22 total ZGP epitopes identified, only 4 have been previously reported. Moreover, only 1 of the 20 MGP and 1 of 16 SGP epitopes were previously described. As such, this the most comprehensive report of preclinical GP epitopes to date, describing GP epitopes from multiple filoviruses in two different mouse genetic backgrounds.

Another novel finding resulting from these analyses was the assessment of the vaccine-induced subdominant T-cell responses, which we show comprised a significant percentage of the total T-cell response, widely ranging between 12 and 74%). Thus, it may be informative in the future to determine the specific contributions of the subdominant and immunodominant epitopic T-cell responses to protection. Notably, these responses may have otherwise been overlooked using traditional matrix array peptide pools for epitope identification. As such, limited epitope detection in previous studies may have been directly related to lower levels of vaccine-induced immunity, the use of less sensitive standard assays, and/or the use of peptide arrangements and/or algorithms favoring detection of immunodominant CD8⁺ epitopes.

Although immune correlates of protection against the filoviruses remain controversial, data generated by this highly immunogenic approach provide a unique opportunity with which to study T-cell immunity as driven by a protective vaccine. E-DNA vaccination herein induced strong ZGP-specific T cells, a large part of which were characterized by Th1-type multifunctional CTL expressing high levels of T-bet, also shown to correlate with T-cell cytotoxicity in humans. It is clear that previous stand-alone DNA vaccine platforms capable of generating mainly humoral immune responses and cellular immunity skewed towards CD4⁺ T cells may likely benefit from in vivo electroporation delivery which has been recently demonstrated to induce potent CD8⁺ T cells in nonhuman primates and the clinic. Thus, data herein support further evaluation of this approach as a stand-alone or prime-boost modality in nonhuman primate immunogenicity and efficacy studies. Specifically, the induction and composition of the CD4⁺ and CD8⁺ effector T-cell response, capacity for T-cell cross-reactivity among divergent GP, and expression of cytolytic function should be explored. This approach offers an attractive vaccination strategy that can be quickly and inexpensively modified and/or produced for rapid response during Filoviridae bio-threat situations and outbreaks. In addition, this model approach provides an important tool for studying protective immune correlates against filoviral disease and could be applied to existing platforms to guide future strategies including HIV.

### Example 42

The present study describes a novel approach for RT-PCR amplification of HIV antigens. Previously, RT-PCR amplification of autologous viral sequences has been confounded by the high mutation rate of the virus which results in unreliable primer-template binding. To resolve this problem we developed a multiplex RT-PCR strategy that allows reliable strain-independent amplification of highly polymorphic target antigens from any patient and requires neither viral sequence data nor custom-designed PCR primers for each individual. It is demonstrated herein the application of our RT-PCR process to amplify translationally-competent RNA encoding regions of Gag, Vpr, Rev and Nef. The products amplified using this method represent a complex mixture of autologous antigens encoded by viral quasispecies. It is further demonstrated that DCs electroporated with in vitro-transcribed HIV RNAs are capable of stimulating poly-antigen-specific CD8⁺ T cell responses in vitro.

This study describes a strategy to overcome patient to patient viral diversity enabling strain-independent RT-PCR amplification of RNAs encoding sequence divergent quasi-species of Gag, Vpr, Rev and Nef from small volumes of infectious plasma. The approach allows creation of a completely autologous therapy that does not require advance knowledge of the HIV genomic sequences, does not have yield limitations and has no intact virus in the final product. The simultaneous use of autologous viral antigens and DCs may provoke broad patient-specific immune responses that could potentially induce effective control of viral loads in the absence of conventional antiretroviral drug therapy.

Immunotherapeutic strategies for HIV-infected individuals are focused on eliciting antiviral CD8⁺ T cell responses to control the level of HIV virus in vivo. Evidence that cellular immune responses play an important role in controlling HIV infection is supported by several observations including: a) Frequencies of CTL inversely correlate with HIV plasma levels b) Blocking CD8⁺ T cells with anti-CD8-specific antibodies in SIV-infected macaques correlates with loss of viral control, c) resolution of acute viremia in the SIV macaque model requires circulating CD8⁺ T, d) The presence of virus-specific CTL coincides with the appearance of mutant viruses which are no longer recognized by these CTL, and e) The presence of CD8⁺CD28⁺ T cells are reportedly associated with long-term non-progression.

Many current HIV immunotherapies utilize individual consensus antigens or defined epitopes derived from those reference HIV sequences. Potential therapies based on clade-specific consensus antigens have been investigated in over 80 clinical trials, however, the results demonstrate a consistent lack of efficacy. While augmentation of immune responses to consensus sequences used for immunization was demonstrated, these therapies did not result in reduction of viral loads. Evidence suggests that the lack of HIV-protective immunity is attributed to sequence divergence between autologous and consensus antigens. The high HIV mutation rate results in novel variants which encode point mutations within CTL epitopes and escape recognition by specific T cells. Studies with overlapping peptides confirmed that CTL recognizing autologous peptides encoded within a known HIV virus did not cross react with corresponding consensus sequences. Studies on humans and non-human primates correlate virus escape from CTL with progression to AIDS. In addition, each patient creates a unique environment for its own viral evolution. Consequently, there is substantial mutational variation between the virus infecting the patient and the reference sequences upon which most HIV immunotherapies are based. Also, since virus sequence diversity defines HIV clades, therapies based on consensus antigens from one clade may have limited ability to cross control evolutionally divergent viruses from other clades. Therefore, therapies based on autologous viral antigens would have broader applicability since the therapy would be perfectly matched to the virus species infecting each subject. To date, the only successful immunotherapies for HIV-infected patients used dendritic cells loaded with autologous viral antigens. Independent clinical studies by Lu et al., and Garcia et. al. demonstrated for the first time that immunization with inactivated whole autologous HIV virus-loaded DC therapy can lead to durable control of viral load. Although these clinical studies demonstrated the potential utility of an autologous DC therapy, the choice of whole inactivated HIV virus as an immunogen is not ideal and may have significant safety and practical limitations.

Described herein is the production in vitro of programmed immune cells comprising: obtaining hematopoetic, mesenchymal, connective tissue, endothelial, cardio, induced pluripotent, osteo, muscle, and/or soft muscle tissue stem cells from a subject; expanding the stem cells from a culture; differentiating the stem cells and; exposing the differentiated stem cells to anthrax particle to generate immune response triggering surface antigen expression in the differentiated cells; and combining said cells so as to comprise a mixture of differentiated cells obtained from about 46% hematopoetic stem cells, about 35% mesenchymal stem cells, about 18% connective tissue, endothelial, cardio, induced pluripotent, osteo, muscle, and/or soft muscle tissue stem cells.

In the present study it is reported that strain-independent RT-PCR amplification of four HIV antigens to generate templates for in vitro-transcribed RNA. Previously a major obstacle to RT-PCR amplification of autologous viral sequences was designing functional PCR primers due to the high mutation rate of the virus. To resolve this problem, we developed a multiplex RT-PCR strategy that allows reliable strain-independent amplification of highly polymorphic target antigens from any patient without the requirement for first knowing the viral sequence and custom-designing of PCR primers for each individual. The amplified products contain a complex mixture of autologous antigens encoded by viral quasi-species. It is further demonstrated that in vitro-transcribed RNA can be delivered to DCs where the encoded antigens are expressed, processed, and presented by MHC class I molecules on the cell surface.

Regions for Gag, Vpr, Rev and Nef were selected for amplification to generate this completely autologous RNA-transfected DC therapy which lacks infectious virus in the final formulation, thereby circumventing potential safety concerns. The choice of antigens targeted for amplification was based on the several criteria: a) Substantial regions of the target gene had to be amenable to PCR amplification using our primer design strategy, b) Antigen expression should not adversely affect dendritic cell function and c) Antigens had to induce functional CTLs. The simultaneous use of autologous viral antigens and DCs may provide for a broad patient-specific immune response that could potentially provide better control of residual virus or rebound of virus following the cessation of antiretroviral therapy.

Amplification of specific HIV genome regions is complicated by the high sequence diversity of the HIV genome. This sequence diversity prevents the design of a single universal primer pair for each gene of interest. To overcome this, we designed pools of forward and reverse primers for each target gene (i.e., Gag, Rev, Vpr and Nef) such that most virus strains will react with at least one forward and one reverse primer. This strategy provides for reliable amplification of intended target antigen genes, as well as the co-amplification of existing HIV quasi-species. A list of individual primers is given in Table 1 (below; SEQ ID NO: 11-50) and the composition of primer groups is given in Table 2 (below). The number of amplification reactions for each HIV antigen was as follows: 6 for Gag, 4 for Vpr, 3 for Rev, and 2 for Nef. The four antigens were amplified from archived frozen plasma infected with diverse clades of HIV: B, C and AG. 2-3 mL of plasma were used to isolate HIV RNA and the titers of these three samples were of 53,334, 53,703 and 154,882 copies/mL, respectively. 2.5 μL of each eluted RNA was used in an RT-PCR for each antigen irrespectively of the initial viral load. PCR resulted in a productive amplification of DNA fragments of expected size for each antigen from all three samples.

TABLE 1

| Primer Name | Primer Sequence |
|---|---|
| GAG F 124 | ACTCTGGTAACTAGAGATCC |
| GAG F 304 | AATTTTGACTAGCGGAGGC |
| GAG F 334 | AGATGGGTGCGAGAGCGT |
| GAG F 334.1 | AGATGGGTGCGAGACCGT |
| GAG R 1881 | GCTCCTGTATCTAATAGAGC |
| GAG R 1881.1 | GCTCCTGTATCTAATAAAGC |
| GAG R 1881.2 | GCTCCTGTATCTAACAGAGC |
| GAG R 1913 | TTTGGTTTCCATCTTCCTGG |
| GAG R 1913.1 | TTTGGTTTCCATCTTCCTGG |
| GAG R 1913.2 | TTTGGCTTCCATCTCCCTGG |
| GAG R 1913.4 | TTTGGTTTCCATTTCCCTGG |
| GAG R 1913.5 | TTTGGTTTCCATTTTCCTGG |
| VPR F 4995 | GCAGGACATAACAAGGTAGG |
| VPR F 4995.4 | GCAGGACATAACAAAGTAGA |
| VPR F 5058 | AAGATAAAGCCACCTTTGCC |
| VPR R 5507 | TTCTTCCTGCCATAGGAGATGC |
| VPR R 5507.1 | TTCTTCCTGCCATAGGAAATGC |
| VPR R 5419 | GCAGTTGTAGGCTGACTTCC |
| VPR R 5419.1 | GCAGITGIAGGCTGACTCCC |
| VPR R 5419.2 | GCAGTTGTAGGCTGGCTTCC |
| REV F 7750 | GGGATTTGGGGTTGCTCTGG |
| REV F 7750.1 | GGGATTTGGGGCTGCTCTGG |
| REV F 7830 | TGATAGTAGGAGGCTTGGTAGG |
| REV F 7830.1 | TGATAGTAGGAGGCTTAATAGG |
| REV F 7830.2 | TGATAGTAGGAGGCTTGATAGG |
| REV F 7911 | GTTAGGCAGGGATATTCACC |
| REV F 7911.1 | GTTAGGCAGGGATACTCACC |
| REV R 8300 | CCCTGTCTTATTCTTCTAGG |
| REV R 8300.1 | CCCTGTCTTATTCTTACAGG |
| REV R 8300.2 | CCCTGTCTTATTCTTGTAGG |
| NEF F 8235 | TAGCTGAGGGGACAGATAG |
| NEF F 8235.1 | TAGCTGAGGGAACAGATAG |
| NEF F aZ35.2 | TAGCTGGCTGGACAGATAG |
| NEF F 8343 | ATGGGTGGCAAGTGGTCAAAAAG |
| NEF F 8343.1 | ATGGGTGGCAAGTGGTCAAAACG |
| NEF F 8343.2 | ATGGGTGGCAAATGGTCAAAAAG |
| NEF F 8343.3 | ATGGGTGGCAAGTGGTCAAAAGG |
| NEF R 9069 | CCAGTACAGGCAAAAAGC |
| NEF R 9069.1 | CAGTACAGGCGAAAAGC |
| NEF R 9069.2 | CAGTACAGGCAAGAAGC |

TABLE 2

| | | | |
|---|---|---|---|
| GAG F 124 | VPR F 4995 | REV F 7750 | NEF F 8235 |
| GAG F 124 | VPR F 4995 | REV F 7750 | NEF F 8235 |
| GAG F 304 | VPR F 4995.4 | REV F 7750.1 | NEF F 8235.1 |
| GAG F 304 | VPR F 5058 | REV F 7830 | NEF F 8235.2 |
| GAG F 334 | VPR F 5058 | REV F 7830 | NEF F 8343 |
| GAG F 334 | VPR R 5507 | REV F 7830.1 | NEF F 8343 |
| GAG F 334.1 | VPR R 5507 | REV F 7830.2 | NEF F 8343.1 |
| GAG R 1881 | VPR R 5507.1 | REV F 7911 | NEF F 8343.2 |
| GAG R 1881 | VPR R 5419 | REV F 7911 | NEF F 8343.3 |
| GAG R 1881.1 | VPR R 5419 | REV F 7911.1 | NEF R 9069 |
| GAG R 1881.2 | VPR R 5419.1 | REV R 8300 | NEF R 9069 |
| GAG R 1913 | VPR R 5419.2 | REV R 8300 | NEF R 9069.1 |
| GAG R 1913 | | REV R 8300.1 | NEF R 9069.2 |
| GAG R 1913.1 | | REV R 8300.2 | |
| GAG R 1913.2 | | | |
| GAG R 1913.4 | | | |
| GAG R 1913.5 | | | |

Summary of samples from 33 patients with diverse viral load from which all four antigens were amplified is given on Table 3 (below). Whenever possible, a greater volume of viral plasma was used for HIV RNA extraction to achieve higher yields of viral RNA. The sample with the lowest viral load examined was sample 1. For this sample the first cDNA synthesis reaction contained 2220 HIV RNA copies. Due to the multiplex design of the amplification, the RT reaction is divided into multiple PCR reactions. Since the Gag single strand cDNA is divided between 6 PCR reactions, the actual copy number in each PCR reaction is 370. A similar result was obtained for sample 3 with a higher viral load but smaller volume of available plasma. For this sample, each RT reaction contained 1580 copies of RNA and each PCR reaction for Gag contained 263 copies of cDNA. The calculation of final recovered HIV RNA concentration assumes no loss during the extraction procedure. With losses, the absolute copy requirement would be even lower. Overall these data demonstrate consistently successful amplification of all four antigens from plasma samples with diverse viral loads.

TABLE 3
Table 3

| sample | viral load | volume of | | coles of RNA |
|---|---|---|---|---|
| number | RNA/mL | plasma used | clade | per RT reaction |
| 1 | 7,413 | 3 | B | 2220 |
| 2 | 14,791 | 3 | B | 2220 |
| 3 | 15,849 | 2 | C | 1560 |
| 4 | 16,596 | 3 | B | 2480 |
| 5 | 18,197 | 3 | B | 5460 |
| 6 | 22,155 | 3 | B | 3320 |
| 7 | 22,909 | 2 | AG | 2300 |
| 8 | 28,840 | 3 | B | 8640 |
| 9 | 33,884 | 3 | B | 10160 |
| 10 | 38,663 | 3 | B | 5800 |
| 11 | 38,905 | 3 | B | 5840 |
| 12 | 45,709 | 3 | B | 6860 |
| 13 | 48,627 | 1 | Nd | 2440 |
| 14 | 50,070 | 1 | Nd | 2500 |
| 15 | 50,119 | 3 | B | 7520 |
| 16 | 50,119 | 3 | B | 7520 |
| 17 | 53,334 | 2 | B | 5340 |
| 18 | 53,703 | 2 | C | 5360 |
| 19 | 53,725 | 3 | B | 8060 |
| 20 | 72,865 | 1 | Nd | 3640 |
| 21 | 72,978 | 1 | nd | 3640 |
| 22 | 95,637 | 2 | B | 9560 |
| 23 | 117,490 | 3 | B | 17620 |
| 24 | 131,826 | 3 | B | 19780 |
| 25 | 134,000 | 3 | B | 20100 |
| 36 | 146,148 | 1 | B | 7300 |
| 27 | 154,882 | 3 | AG | 23240 |
| 28 | 158,489 | 3 | B | 23780 |
| 29 | 244,000 | 1 | B | 12200 |
| 30 | 513,000 | 3 | Nd | 38480 |
| 31 | 1,138,560 | 3 | B | 170780 |
| 32 | 1,479,108 | 3 | Nd | 221860 |
| 33 | 3,221,835 | 1 | B | 161100 |

An advantage of this approach for antigen generation is its ability to capture HIV mutations which evolve under dynamic host CTL pressure. It is broadly applicable to the general HIV-infected population irrespective of Clade designation, but also anticipates that it would capture various quasi-species present in a given subject. This novel therapeutic paradigm enables targeting, not only of dominant viruses, but also newly emerging virus populations which evolve as a result of immune pressure.

To test the hypothesis that multiple quasi-species are co-amplified from a given subject, PCR fragments encoding full length Nef cDNA amplified from Clade B samples HTM-349, HTM-367 and HTM-344 (viral load 513,000; 53,334 and 95,637 copies per mL respectively) were cloned, sequenced and analyzed using phylogenetic tree analysis. A total of 15 clones were analyzed for each subject. The analysis demonstrated that the cDNA population did indeed capture genes encoded by various HIV quasi-species. Phylogeneic tree analysis demonstrated that each subject's Nef sequences grouped within other sequences from that subject and were distinct from another subjects' sequences. More interestingly however is the observation that the number of the Nef variable sequences differed in each subject. At the nucleotide level the subject HTM 344 displayed greater diversity where out of 15 clones analyzed, 14 clones were unique, followed by subject HTM 367 with 13 unique clones and for subject HTM 349 only 6 unique clones. The subject-specific sequence clustering together with the variable number of unique clones between patients eliminates the possibility that the mutations are random mutational artifacts introduced during RT-PCR. Not every nucleotide mutation leads to an amino acid substitution, so the diversity is lower at the level of amino acid sequence with the same order of diversity trend for the three subjects. Similar analyses were performed for cDNAs encoding Gag, Rev and Vpr cDNA amplified from various subjects. These data indicate that, as predicted, the multiplex RT-PCR is capable of capturing various quasi-species within each individual subject. Next further analysis of the Nef sequences was performed and scored individual primers on the level of productive secondary PCR reactions. Since the same formulation of primers was used with all samples independent of clade, we were interested to learn which primers within the formulated groups were leading to productive amplification and analysis of preferential use of forward and reverse primers was performed (Table 4; below; SEQ ID NOs: 51-57). The forward primer utilization of sample HTM 344 demonstrated that 12 out of 15 clones were formed by primer F8343.1, however in sample HTM 367 a different primer, F8343 formed the majority of clones. Interestingly, a novel primer annealing sequence was identified. The sequence was termed “new” and differed from either F8343 or F8343.1 primer sequence by 2 nucleotides in the most 3′ position. We believe that the new sequence was formed due to the 3′ exonuclease proof-reading activity of the PCR enzyme. Similarly, different preferences for use of reverse primers were observed although no new reverse primers were identified in all three groups analyzed. The 3′ end of the PCR fragment is defined by the cDNA synthesis step during the RT reaction, and the lack of “repaired” primers is most likely due to lack of proofreading activity in the RT enzyme. Similar analyses were performed on multiple Rev clones and confirmed the original observation (data not shown). Since the sequence within regions of interest varies from patient to patient, the preferred utilization of the primers in the PCR reactions differ as well.

TABLE 4

| | | Utilization in HT-PCR | | |
|---|---|---|---|---|
| Primer | Primer sequence | in various subjects | | |
| name | 5′-3′ | HTM 344 | HTM 349 | HTM 367 |
| T7 Nef F | TAATACGACTCACTA | 1 | 0 | 10 |
| 8343 | TAGGGAGACCACCAT | | | |
| | GGGTGGCAAGTGGTC | | | |
| | AAAAAG | | | |
| T7 Nef F | TAATACGACTCACTA | 12 | 0 | 0 |
| 8343.1 | TAGGGAGACCACCAT | | | |
| | GGGTGGCAAGTGGTC | | | |
| | AAAACG | | | |
| New | TAATACGACTCACTA | 2 | 15 | 5 |
| | TAGGGAGACCACCAT | | | |
| | GGGTGGCAAGTGGTC | | | |
| | AAAAGG | | | |
| | TAATACGACTCACTA | | | |
| | TAGGGAGACCACCAT | | | |
| | GGGTGGCAAGTGGTC | | | |
| | AAAAAT | | | |
| 64T Nef R | (T)₆₄CCAGTACAGG | 7 | 8 | 11 |
| 9069 | CAAAAAGC | | | |
| 64T Nef R | (T)₆₄CAGTACAGGC | 6 | 2 | 4 |
| 9069.1 | GAAAAGC | | | |
| 64T Nef R | (T)₆₄CCAGTACAGG | 2 | 5 | 0 |
| 9069.1 | CAAGAAGC | | | |

The Nef (Table 4) cDNA sequences were analyzed in the regions corresponding to the regions defined by the primers and identity of the primers was identifies by sequence. Total of 15 Nef clones were analyzed for subjects HTM344, HTM 349 and HTM 367. The number in the right three columns represents how many clones contained the identified primer.

The goal of active HIV immunotherapy is to stimulate the preferential expansion of antiviral effector T cells. To demonstrate that HIV RNAs generated by the approach can express antigens capable of inducing CD8⁺ T-cell immunity, DC electroporated with all four autologous HIV antigens encoded as RNAs were prepared. 1 μg Gag RNA, 0.25 μg Nef RNA, 1 μg Rev RNA, and 1 μg Vpr RNA were electroporated along with 1 μg of CD40L RNA per 10⁶ DC. Since cells were fully matured by overnight incubation in the presence of TNFα, INFγ and PGE₂ the maturation status of the DCs did not change after electroporation with the RNAs. These cells were co-cultured with autologous PBMCs pre-labeled with CFSE. After 6 days of co-culture, the frequency and phenotype of proliferating cells was detected by residual CFSE fluorescence within the CD8⁺ T cell population with effector (CD45RA⁺/CD28⁻) or effector/memory (CD45RA⁻/CD28⁺) phenotypes. After 6 days of co-culture, the CD8⁺ T-cell population was stimulated with either eGFP RNA-transfected DC (negative control) or HIV RNA-transfected DC. The frequency of CFSE-low cells stimulated with GFP RNA-loaded DC was 3.75% while those stimulated with HIV RNA-loaded DC had a frequency of 7.41%. No proliferation above the negative control background was observed within the CD4⁺ T cell subset, with all DC populations inducing 1% CD4+CFSE-low cells within total PBMCs. Within the proliferating CFSE-low CD8⁺ T cell subset stimulated with HIV RNA-loaded DC, 24.7% of cells exhibited a phenotype consistent with fully differentiated effector cells (CD8⁺CD28⁻CD45RA⁺) versus 54.8% of cells had a phenotype indicative of effector/memory cells (CD8+CD28+CD45RA−).

To assess the specificity and effector function of the T cells responding with antigen-induced proliferation, the cultures were further re-stimulated with either DC electroporated with GFP RNA (negative control), a pool of all 4 HIV RNAs, or each HIV RNA independently. After 4 hours CFSE-low CD8⁺ T cells were tested for IFN-γ expression by intracellular staining. Induction of IFN-γ expression above the GFP negative control background was observed for all HIV RNA DC-stimulated cultures. Co-cultures that were originally established with GFP RNA-electroporated control DC for 6 days and then restimulated with individual HIV antigen-encoding RNA-electroporated DC all expressed less than 0.15% IFN-γ activity within the CD8+ CFSE-low subset (data not shown).

Sequences of HIV isolates were aligned using BLAST analysis with sequence. Nucleotide regions which appeared to have sequence conservation were selected for primer design. Alternative primers which could also accommodate the frequently found mutations in were designed as well. These alternative primers contain compensatory sequence variations to accommodate the frequently found mutations in positions 7847 and 7848 (Rev F 7830.1 and Rev F 7830.2).

To minimize the number of primers used, a mismatch at the 5′ end of a primer sequence was tolerated since lowering the PCR amplification stringency could compensate for such mismatches. However, mismatches at the 3′ end of a primer sequence were avoided.

To enable transcription of the PCR product in vitro, the products of the primary PCR reaction were modified to insert a T7 RNA polymerase binding site at the 5′ end. Naturally occurring translation initiation codons for Gag, Vpr and Nef were captured during PCR amplification. However Rev mRNA is formed in a transplicing event and capture of a full length cDNA via PCR is not achievable. Only the second exon of Rev is amplified, so the addition of the initiator ATG codon for the Rev antigen in a nested round of PCR is required in order to enable translation initiation. The reverse primers contain a poly(T)₆₄ tail which is transcribed into a poly(A)₆₄ tail on the synthesized RNAs. Individual primer sequences for the primary round of amplification are provided in Table 1.

Oligonucleotides (IDT) were reconstituted at a concentration of 100 mM. Primers were combined into groups to reduce the number of PCR reactions (the composition of primer groups is provided in Table 2. The final primer concentration in formulated stock solutions was 5 M for PCR, and 20 μM for gene-specific reverse transcription. The amplification protocol was simplified by grouping primers according to their location. The number of amplification reactions for each HIV antigen was significantly reduced from the scenario where individual primer combinations would be used: 6 for Gag, 4 for Vpr, 3 for Rev, and 2 for Nef. Once primer mixes were made they were not further changed and the same formulations of primers were used to amplify various plasma materials.

HIV RNA was isolated from 1 to 3 mL of plasma from HIV patients using a NucliSens kit (BioMerieux), according to the manufacturer's instructions and eluted in 30 μL of nuclease free water. First strand cDNA synthesis reaction contained gene-specific primers for either Gag, Vpr or Rev, and oligo dT₍₂₀₎ (Invitrogen) for Nef, 40 units of RNAseOut (Invitrogen), 0.5 mM of each dNTP (Clontech), and Superscript first strand buffer. After annealing at 65° C. for 5 minutes, DTT to 5 mM and 400 units of Superscript III (Invitrogen) were added and the reaction was incubated at 55° C. for 1 hour.

2.5 μL of the first strand cDNA reaction was then taken into a primary PCR reaction containing 5 units of PFU ultra HS, PFU buffer (Stratagene), 0.2 mM of each dNTP (Stratagene), and the corresponding group of primers at a final concentration 0.4 M for Gag, 0.6 M for Vpr, 0.2 μM for Rev, and 0.4 μM for Nef, in a final reaction volume of 50 μL. The PCR reaction was denatured at 95° C. for 2 minutes and then run for 40 cycles as follows: 95° C. for 30 seconds, 54° C. for 30 seconds, and 72° C. for 3 minutes and 10 minutes for the last cycle. The annealing temperature was kept at 54° C. here and in the secondary PCR amplification to allow for annealing of primers to templates with a limited degree of mismatch.

1 μL of the primary PCR reaction was then taken into a secondary PCR reaction containing 2.5 units of PFU Ultra HS, PFU buffer (Stratagene), 0.2 mM of each dNTP (Stratagene), and gene specific T7 and 64T groups of primers, in a final reaction volume of 25 μL. The cycling parameters were the same as in the primary PCR reaction. Products of the secondary PCR reaction were purified using a QIAquick purification column (QIAGEN). For preparative PCR several (i.e. 6 or 12) reactions were established with same conditions as secondary PCR except the total volume of each reaction was 50 μL.

Gag, Rev and Vpr were amplified from plasmid pBKBH10S and Nef was amplified from plasmid p93TH253.3 obtained from NIH AIDS Research & Reference Reagent program. Single forward and reverse PCR primers were selected with full complementarity to the template determined by sequence analysis. All PCR conditions were exactly the same as used for amplification of the infectious material.

Secondary PCR fragments served as templates for an in vitro transcription reaction using mMessage mMachine T7 Ultra kit (Ambion) according to the manufacturer's instructions. The amplified RNA was purified using RNeasy columns (QIAGEN).

A leukapheresis sample from a volunteer was collected on a COBE Spectra (Gambro BCT) using the AutoPBSC procedure described by Lifeblood (Memphis, Tenn.). Peripheral blood mononuclear cells were isolated using a Ficoll density gradient (Histopaque®-1007 Hybri-Max®, Sigma) and cultured for 1 to 2 hours to allow adherence of the monocytes. Non-adherent cells were removed and the remaining monocytes were cultured in X-VIVO 15™ (Cambrex) medium for 6 to 7 days, supplemented with 1000 U/mL each of GM-CSF (Berlex, Leukine® liquid) and IL-4 (R&D Systems).

Immature DCs were generated as described above from a successfully HAART-treated HIV donor with a viral plasma copy number of less than 200 copies per mL. To achieve DC maturation, immature DC were cultured on day 5 with 10 ng/ml TNF-α, 1000 μg/ml IFN-γ, 1 μg/ml PGE₂. On Day 6, matured DCs were co-electroporated with in vitro transcribed RNA encoding CD40L at 1 μg per million of DC, and 1 μg Gag, Rev, Vpr and 0.25 μg Nef autologous HIV RNAs per million of DCs. A negative control DC stimulator was generated by transfecting DC with CD40L RNA and 3.25 μg eGFP RNA, instead of HIV RNA mix. RNA-electroporated DC were further cultured for 4 hrs in X-VIVO-15 medium without additional cytokine supplements.

PBMCs from the HIV donor were enriched by Ficoll gradient separation, washed twice with PBS and re-suspended at 2.0×10⁷ cells per mL in PBS. CFSE was added to the cell suspension for a final working concentration of 1.0 μM and incubation for 8 minutes at room temperature. The staining was quenched by the addition of an equal volume of Human AB Serum and incubation for 2 minutes.

Cultures of HIV RNA-electroporated mature DC, and eGFP-RNA control DC were established in parallel with CFSE-labeled PBMC at a 1:10 ratio, 1 million total cells/mL in 5% Human AB serum for 6 days at 37° C., 5% CO₂.

After 6 days of culture, PBMCs were harvested, washed once with 2 mL PBS containing 10% FBS and stained for surface antigens using CD45RA PE, CD8 PerCP-Cy5.5, CD28 APC or CD45RA PE, CD4 PerCP-Cy5.5, CD28 APC antibodies (BD Bioscience) at room temperature in the dark for 20 minutes. Samples were washed twice with cold PBS containing 10% FBS and re-suspended in 300 μL of 2% BD Cytofix (BD Bioscience) Samples were acquired on a BD FACSCalibur flow cytometer and analysed using FlowJo software (Three Star, Inc.) Analysis gates were set on the basis of FSC v. SSC to define viable lymphocytes and lymphoblasts, and the frequency of proliferating cells determined by detection of CFSE ‘low’ cells, and their associated cell surface phenotype.

Immature DC were generated as described above, matured with TNF-α, IFN-γ and PGE₂ and cells split into 5 groups, allowing for DC populations to be generated expressing just a single HIV gene from the panel of four individual antigens, and a fifth DC population electroporated with eGFP RNA, as negative control. The DC populations were co-cultured in parallel with CFSE-labeled PBMC harvested from the previous 6-day co-culture described above. One hour after re-stimulation with DCs, 0.25 μl of Golgi plug (BD Bioscience) was added to each sample and incubated for an additional 3 hours at 37° C., 5% CO₂ in RPMI containing 10% Human Serum. Samples were washed once with 1 ml PBS containing 10% FBS and stained for surface antigens using CD8 PerCP-Cy5.5 or CD4 PerCP-Cy5.5 antibodies at 4° C. in the dark for 20 minutes. After wash with PBS containing 2% FBS and re-suspension in 150 μl of 2% BD Cytofix cells were incubated at room temperature in the dark for 20 minutes. Samples were washed twice with 1 ml of Perm/Wash buffer (BD Bioscience) and incubated at room temperature in the dark for 20 minutes with 2 μl of purified Mouse IgG₁ antibody. Samples were stained for intra-cellular cytokines using IL-2 PE and IFN-γ APC antibodies at room temperature in the dark for 20 minutes. Samples were washed twice with 1 ml of BD Perm/Wash buffer and re-suspended in 150 μl of 2% BD Cytofix, acquired on a BD FACSCalibur flow cytometer and analyzed using FlowJo software. PBMC that had proliferated (CFSE ‘low’) during the previous 6-day co-culture were gated and analyzed for induced IFN-γ and IL-2 content.

### Example 43

Smallpox, because of its high case-fatality rates and transmissibility, now represents one of the most serious bioterrorist threats to the civilian population. Smallpox is considered a category A biological disease. Category A biological diseases are considered high-priority agents that include organisms that pose a risk to national security because they: can be easily disseminated or transmitted person-to-person; cause high mortality, with potential for major public health impact; might cause public panic and social disruption; and require special action for public health preparedness.

Smallpox is one of these such organisms. Smallpox infection was eliminated from the world in 1977. Smallpox is caused by variola virus. The incubation period is about 12 days (range: 7 to 17 days) following exposure. Initial symptoms include high fever, fatigue, and head and back aches. A characteristic rash, most prominent on the face, arms, and legs, follows in 2-3 days. The rash starts with flat red lesions that evolve at the same rate. Lesions become pus-filled and begin to crust early in the second week. Scabs develop and then separate and fall off after about 3-4 weeks. The majority of patients with smallpox recover, but death occurs in up to 30% of cases. Smallpox is spread from one person to another by infected saliva droplets that expose a susceptible person having face-to-face contact with the ill person. Persons with smallpox are most infectious during the first week of illness, because that is when the largest amount of virus is present in saliva. However, some risk of transmission lasts until all scabs have fallen off.

The eradication of smallpox occurred prior to modern advances in virology and immunology, precluding a thorough understanding of the basis for protection following vaccination. The vaccines used in the global smallpox eradication campaign consisted of several related strains of live VACV (Dryvax® New York City Board of Health strain in the USA; Lister in the UK; Temple of Heaven in China, and EM-63 in the USSR) usually administered percutaneously by scarification of the skin with a bifurcated needle or with a jet injector. Smallpox vaccine recipients with severe T-cell abnormalities developed generalized VACV infection, whereas agammaglobulinemics did not, pointing to the importance of cell-mediated immunity in controlling the primary infection caused by the live vaccine. A successful vaccination or ‘take’ in a naïve, immunocompetent individual results in VACV replication in the skin producing a papule with surrounding erythema in 3 to 5 days, followed a few days later by a vesicle and then a pustule. A scab forms and separates from the skin after 2 to 3 weeks. Low-grade fever, headache, myalgia, fatigue, and regional lymphadenopathy often accompanies vaccination and correlates with increased levels of cytokines. A modified or accelerated skin reaction is usually indicative of pre-existing immunity. Protection against mortality due to smallpox is nearly complete for 20 to 30 years and gradually wanes thereafter.

In naïve individuals, a vaccine take correlated with development of neutralizing antibody to VACV, whereas in previously vaccinated individuals successful takes correlated inversely with the pre-existing neutralizing antibody levels. A prospective study showed that smallpox contacts with a neutralizing antibody titer of 1:32 or greater were protected against disease. The route of administration of the vaccine is important: the percentage of children developing neutralizing antibody after receiving the vaccine percutaneously and subcutaneously was 83 and 23%, respectively, and this difference correlated with immunity to revaccination. In another study, percutaneous administration produced a higher neutralizing antibody response than intradermal or intramuscular administration. Neutralizing antibody was detected by days 12 to 15 and was maximal by days 25 to 30. Recent studies indicate that CD4⁺ and CD8⁺ T cells are also induced by smallpox vaccination. Studies of elderly individuals demonstrated that the antibody levels determined by enzyme-linked immunosorbent assay (ELISA) or neutralization of VACV persist for many decades, as do memory B and CD4⁺ and CD8⁺ T cells. Multiple vaccine boosts, however, do not seem to enhance the long-term antibody levels. Although not carried out in a controlled manner, several studies suggest that administration of hyperimmune globulin (VIG) provided significant protection to smallpox contacts and alleviation of vaccine complications. In summary, the data show a positive correlation of vaccine-mediated protection against smallpox with a skin take, neutralizing antibody, and VACV-specific T cells.

Interest in smallpox vaccines was renewed in recent years due to concerns that a still existing unregistered stock of VARV could be used as a bioweapon, particularly since the unvaccinated population is now fully susceptible to infection. Stocks of smallpox vaccine dwindled in the years following eradication and their replenishment has been advocated. However, the classical method of vaccine production in the skin of animals does not meet current safety standards. In addition, although the vaccine was considered safe when contrasted with the possibility of contracting smallpox, significant numbers of serious adverse events were recorded. A safer vaccine could benefit millions of people advised not to take the classical one because they or their contacts have immune deficiencies, eczema, or atopic dermatitis, which make them more susceptible to generalized or progressive vaccinia. In certain situations, a safer vaccine might also be used to prevent disease caused by other OPXVs. Monkeypox virus (MPXV) is a continuing zoonosis in parts of Africa and a potential terrorist agent. A mild form of MPXV infected 69 people in the United States following importation of African rodents, which comprise the natural reservoir. Self-limited human infections with several strains of CPXV transmitted by rats and cats occur in Europe and with feral vaccine-derived VACV in India and Brazil.

Since smallpox has been eradicated, the efficacy of new vaccines must be evaluated indirectly by an analysis of human immune responses supplemented with the use of animal models and surrogate poxviruses to demonstrate disease protection. An understanding of the biology of poxviruses and the targets of the protective immune response can facilitate the development of new generation vaccines. For this reason, the present review includes an outline of the biology of OPXVs with particular relevance to vaccine development, discusses targets of humoral and cellular immunity, compares smallpox with animal models of OPXV disease, evaluates correlates of protection, and provides the status of second and third generation smallpox vaccines. Excellent reviews of smallpox vaccines from different perspectives have been published recently.

Poxviruses comprise a large family of enveloped, double-strand DNA viruses that infect vertebrate and invertebrate species. The members of this family are unusual, with regard to other DNA viruses, in their cytoplasmic site of replication. This review focuses on orthopoxvirus (OPXV) genus, which includes VARV, VACV, CPXV, MPXV, and ectromelia (mousepox) virus (ECTV) among others. OPXV double-stranded DNA genomes are about 200,000 bp in length and encode about 200 proteins, most of which have 90% or more sequence identity between species. The complete genome sequences of a large number of VARV isolates and other OPXVs are available. While all OPXVs encode homologous proteins for vital functions such as entry, gene expression, genome replication, and virion assembly, there are differences with regard to proteins involved in immune evasion and other host interactions. In this respect, CPXV has the most complete genome, whereas some immune evasion and host interaction genes are missing or disrupted in VARV, VACV, MPXV, and ECTV. The similarity in the structural proteins expressed by different OPXVs accounts for their immunological cross-reactivity and protection. The viral DNA polymerase has high fidelity resulting in a low mutation rate, consistent with the absence of notable antigenic variation.

The basic infectious poxvirus particle is the mature virion (MV), which consists of a nucleoprotein core containing the genome and a complete early transcription system, flanked by lateral bodies, and surrounded by a lipoprotein envelope. Mass spectrometry studies indicate that there are more than 80 viral protein components of the MV, of which more than 20 are associated with the surface membrane. The MVs assemble in viral factory regions of the cytoplasm and may remain in the cytoplasm as individual particles or become occluded in dense bodies known as A-Type inclusions in the case of ECTV and CPXV until cell lysis. Free and occluded MVs are thought to be important for transmission between hosts. In addition, a subset of MVs becomes surrounded by modified Golgi or endosomal membranes, transported to the cell periphery on microtubules, and released by exocytosis as enveloped virions (EVs). The EV consists of an MV with an additional lipoprotein membrane containing eight EV-specific viral proteins, although there are other small differences in protein composition. Most of the EVs remain adherent to the plasma membrane and mediate cell-to-cell spread at the tips of long actin-containing microvilli, which are important for virulence in animals. Since the fusion proteins necessary for virus entry are located in the MV, disruption of the EV membrane is required for their exposure. Studies of VACV entry indicate that fusion can occur at the plasma membrane or in endosomes by a fluid uptake or macropinocytosis mechanism.

Four proteins enable attachment of MVs to the cell. The A27 protein exists as trimers and hexamers on the surface of MVs and binds heparin and cell surface proteoglycans. H3, another MV surface protein, also binds heparin and cell surface proteoglycans. D8 is an MV surface protein that binds chondroitin sulfate. The fourth attachment protein, A26, is physically associated with A27 and binds cell surface laminin. Fusion with the cell membrane and entry of the core into the cytoplasm requires at least 12 additional MV transmembrane proteins that form a complex known as the entry fusion complex or EFC. Such a large number of entry proteins are unprecedented among viruses. The entry proteins are A16, A21, A28, F9, G3, G9, H2, 12 (not yet shown to be a component of EFC), J5, L1, L5, and 03. The individual roles of the EFC proteins remain to be determined.

The transmembrane proteins exposed on the outer surface of the EVs include A33, A34, A36, A56 hemagglutinin, and B5. Deletion of any one of the above genes, except for the gene encoding A56, results in decreased virus spread. The A36 protein is particularly important for formation of actin tails. K2 and the complement regulatory protein C3 lack transmembrane domains but are associated with A56. A56 and K2 help to prevent superinfection of cells and cell surface C3 enhances protection against complement. F13, a palmitoylated protein, is associated with the internal surface of the EV membrane and is important for EV formation.

Viral gene expression is stringently regulated during infection. The early transcription system, which is packaged in infectious virus particles, directs the expression of more than half of the genome shortly after entry of the core. Although kinetic analysis reveals two early gene clusters, the members of both are synthesized without the need for de novo protein synthesis and may all be classified as belonging to the immediate early class as defined for other viruses and phage. The genes of both early kinetic classes encode proteins involved in host interactions including immune defense, DNA replication, and gene expression. Transcription of the remaining genes is dependent on viral protein and DNA synthesis and they are grouped in intermediate and late classes. The latter encode most of the proteins assembled into virus particles, which are important targets for neutralizing antibodies. Early, intermediate and late genes have distinct promoter sequences that are recognized by stage-specific transcription factors and a multi-subunit DNA-dependent RNA polymerase. The close spacing of genes and the tendency of transcripts to overlap, particularly at late times after infection, have made global transcript mapping difficult. The problem was only partly overcome by use of tiling arrays, which have been superseded by higher resolution deep RNA sequencing. Further refinements to more definitively map the transcriptional start and stop sites are anticipated. As discussed below, the time of synthesis of proteins may affect their presentation to the immune system.

Not all cells support OPXV replication. Importantly for this review, replication of VACV is abortive in mouse and human primary macrophages and dendritic cells but permissive in B cells. Although VACV is able to enter these non-permissive cells and express early genes, neither viral DNA replication nor late genes are expressed. Unaccountably, intermediate genes are expressed in human macrophages despite the apparent absence of viral DNA replication.

Based on flow cytometry studies of human peripheral blood lymphocytes infected with recombinant VACV expressing green fluorescent proteins regulated by an early/late promoter, the levels of expression were monocytes (CD14⁺)>B lymphocytes (CD 19k)=NK (CD 56⁺)>T lymphocytes (CD3⁺). The block in resting T cells occurs at the virus-binding step, which is overcome upon T-cell activation in vitro. Some virion surface proteins provide accessible targets for antibodies. As indicated above, the situation is complicated for OPXV because of the existence of two main infectious forms. Antigenic differences between the MV and EV forms of VACV were recognized over 40 years ago and correlated with greater protection by live virus compared to inactivated virus. Both live and inactivated vaccines generally consist of MVs isolated by disruption of infected cells and further purified. However, whereas live MVs replicate in the animal host and induce antibody to both forms of virus, inactivated MVs only induce antibody to themselves. Depending on the process, it is also possible that inactivation destroys some immunogens of the MV and that the live virus induces protective antibodies to non-virion components as well as to EVs and also stimulates cellular immunity. No successful inactivated OPXV vaccines have been made.

The traditional way of measuring antibodies that neutralize MVs has been by plaque reduction. MVs are incubated with antibodies and then allowed to form plaques in a few days on a monolayer of susceptible cells. Faster, higher throughput assays have been devised using recombinant MVs that express β-galactosidase or green fluorescent protein. EV neutralization can also be measured by a plaque reduction assay, although EVs are more difficult to neutralize than MVs. One technical problem is that EVs are fragile and the outer membrane is easily disrupted exposing the MV membrane. To alleviate this problem, a monoclonal antibody (mAb) to an MV protein is frequently used to neutralize partially disrupted EVs before adding EV-specific antibody. Alternatively, the ‘comet reduction assay’ is used to measure antibodies that prevent the spread of EVs in cell monolayers. The so-called comets are satellite plaques that form by EVs released from infected cells. Antibodies to EV proteins may reduce the size and number of the comets; however, even high titer antibodies from infected animals cannot prevent cell-to-cell spread as seen by expansion of the primary plaque.

The identification of antibodies to specific MV and EV proteins followed progress in dissecting the structure of VACV. Table 5 lists proteins associated with the MV and EV-specific membranes that have been shown to be targets of neutralizing or comet-reducing antibodies. Polyclonal antibodies to the MV membrane-associated proteins A17, A27, A28, D8, H3, and L1 can neutralize VACV. As indicated above, A27, D8, and H3 are involved in attachment of MVs, and A28 and L1 are required for fusion of the viral and cell membranes. Remarkably, H2, another protein required for fusion, interacts directly with A28 and greatly increases the amount of neutralizing antibody that reacts with A28. However, immunizations of mice with the genes encoding the other VACV entry proteins did not result in the generation of neutralizing antibody.

Neutralizing mouse mAbs that target the A27 and L1 MV proteins have been described. Several neutralizing mAbs target the N-terminal region of A27. Neutralizing mAbs bind to a native conformation-specific form of L1 with intact intramolecular disulfide bonds. Structural studies show that the Fab fragment binds to a discontinuous epitope in L1 consisting of two loops that are held together by a disulfide bond. L1 mAb can neutralize VACV after cell attachment, but the mechanism of neutralization is not understood.

Antibodies to two EV-specific proteins, A33 and B5, inhibit comet formation and B5 antibody also neutralizes EV infectivity. Rodent and human mAbs to the A33 and B5 proteins are capable of neutralizing EV and/or reducing comet formation. The epitopes of these mAbs have not been precisely mapped. However, despite the similar sequences of A33 homologs in OPXV, mAbs can differentiate between the homologs in VACV, ECTV, and MPXV. Although the proteins of VACV and VARV have a high degree of amino acid identity, there are differences in some that may be relevant for vaccine purposes. Thus, VACV B5 and the VARV homolog B6 have 23 amino acid differences. Similarly, although polyclonal antibodies to B5 cross-reacted with the homologous VARV protein, 10 of 16 mAbs did not.

Efforts have been made to more completely characterize the binding antibodies in human or animal sera following immunization with live VACV. In a proteome-wide approach, proteins corresponding to 185 VACV open reading frames were expressed in an Escherichia coli cell-free system and printed on a microarray. The microarray was then incubated with sera from humans, rabbits, or mice that were infected with VACV Antibodies to about 25 proteins, many of which are unlikely to be involved in virus neutralization, were detected with considerable heterogeneity of individual responses. After the first vaccination, antibodies to six membrane proteins, A13, A17, A33, B5, D8, and H3, were found in 50% or more of the 13 individual human sera tested. After the second vaccination, A27 and L1 were detected. An attempt was made to correlate the neutralizing activity in individual sera with the amount of antibody to 16 membrane-associated MV proteins. A positive correlation was found for H3, A27, D8, A14, D13, and L1. However, depletion of either H3 or L1 antibody or both from sera did not reduce the neutralizing activity. This was true even when H3 was depleted from serum that had detectable reactivity to only H3 and D8. Earlier studies had shown that depletion of either L1 or A27 from pooled human sera also did not significantly reduce the neutralizing titer. In a separate study, modest reductions in MV neutralizing activity was obtained by adding A27 and H3 proteins to human immune sera but not by adding L1 protein. These results suggest that there are multiple neutralizing targets on the MV, likely including some that have not yet been identified, or that the targets are complex and comprised of multisubunit structures such as A28-H2 or higher order assemblies.

In contrast to the results with MV neutralization, depletion of B5 from sera greatly reduces in vitroneutralization of EVs indicating that this protein is the major target. Nevertheless, B5 antibody-depleted serum can reduce comet formation indicating that binding antibodies to additional EV proteins are important. Moreover, as discussed below, immunization with the A33 EV protein is highly protective in animal models.

Both classical and alternative complement pathways have been reported to enhance the neutralization of MVs in vitro. Complement can enhance the neutralization of EVs by two different methods. Complement in conjunction with antibody to the A33 protein was shown to disrupt the EV membrane and allow access of MV neutralizing antibody. In addition, complement was shown to enhance B5 antibody neutralization of EV by opsinization and by lysing VACV-infected cells. Depletion of complement in the severe combined immunodeficiency (SCID) mouse reduced the ability of B5 mAb to protect against a VACV infection; in addition, protection was shown to be isotype dependent. The importance of complement in defense against OPXVs is underscored by their encoding a secreted complement regulatory protein that can prevent antibody-dependent complement enhanced neutralization of MV infectivity and is required for virulence. The presence of cellular complement regulatory proteins associated with the EV may similarly provide protection against complement.

Consistent with the large number of OPXV proteins, hundreds of VACV CD8⁺ T-cell epitopes have been identified in humans, monkeys and mice, as recently reviewed. Furthermore, based on sequence comparisons, a high percentage of the VACV epitopes are predicted to be present in VARV. Studies with recombinant VACV demonstrated the importance of early expression for induction of CD8⁺ T cells. Relatively more targets are found in VACV proteins expressed early compared to late in infection and immunoprevalence correlates with mRNA abundance at 4 h after infection. The early gene bias may be correlated with the abortive infection of dendritic cells by VACV, which prevents late gene expression. In addition, cross-competition of CD8⁺ T cells shapes the immunodominance hierarchy favoring early expression during a boost vaccination.

A comprehensive literature compilation and review indicates that 133 VACV proteins are recognized by CD4⁺ T cells, though the epitopes have not been precisely mapped in many cases. In contrast to CD8⁺ T-cell antigens, CD4⁺ T-cell antigens are directed mostly to late virion proteins, as are antibodies.

For an ideal animal model, the disease would closely mimic human smallpox. The transmission of VARV in humans occurred mainly by droplets or aerosol from close contacts. A one to two week incubation period preceded the abrupt onset of fever, headache, and back pain, followed a few days later by eruptive lesions on the tongue and mucous membranes of the mouth and oropharynx and then by maculopapular lesions that spread from the face and extremities and progressed into pustules covering the entire body. The delayed time course is thought to represent initial local replication near the site of infection and in local lymph nodes followed by systemic spread with tropism for the skin.

Since the eradication of smallpox, the World Health Organization has tightly regulated experimentation with VARV. Such studies are permitted at only two sites: one in the United States and the other in Russia. VARV is naturally host-restricted to humans and very high doses are needed to cause pathology in cynomolgus monkeys. Acute pathology and rapid death occurred when 10⁹ plaque forming units (PFU) of VARV were administered intravenously. Virus was detected in lymphoid tissue, skin, oral mucosa, gastrointestinal tract, reproductive system, and liver, correlating with organ dysfunction and multisystem failure. At best, this model mimics the late systemic spread phase of smallpox. Aerosol doses of >10⁸ PFU caused only mild clinical signs. Safety recommendations for other OPXV are described in a publication by the Centers for Disease Control and Prevention and the National Institutes of Health.

Human monkeypox is a zoonosis that resembles smallpox clinically but has lower rates of fatality and human-human transmission and differs from it epidemiologically. In addition, there are differences in the immunomodulatory and host range genes of VARV and MPXV. Despite its name, MPXV is widely distributed in a variety of African rodents, particularly squirrels, which are probably the maintenance reservoir. Two strains of MPXV are now recognized: the more virulent strain with an estimated mortality rate of 10% originates from the Congo Basin in Africa, and the milder strain, which was imported transiently into the United States in 2003, originates from West Africa. Congo Basin strains of MPXV are lethal to cynomolgus monkeys when inoculated by aerosol, intratracheally, subcutaneously, or intravenously with relatively high doses, ranging from 10⁵ to 10⁷ PFU. After respiratory inoculation, death in 9 to 17 days is attributed to bronchopneumonia. MPXV can experimentally infect and cause disease in a variety of wild rodents including ground squirrels, prairie dogs, and African dormice at relatively low doses (<10¹-10⁴ PFU), but these animals are difficult to breed and there is a lack of immunological reagents. Although classical inbred mice are resistant to MPXV, an extensive search revealed three ‘wild-derived’ inbred strains that are highly susceptible to infection. For the CAST/eij mouse strain, the 50% lethal dose of MPXV is 670 PFU by the intranasal route and a log less by the intraperitoneal route, whereas even 10⁷ PFU caused no deaths in BALB/c mice. The CAST mouse/MPXV model may have advantages for studying correlates of immunity and vaccine efficacy.

ECTV is a sporadic infection of laboratory mice. The classic inbred strains vary greatly in their susceptibility with lethality occurring with doses of 1 PFU or less in the most sensitive strains. ECTV has been extensively used to study viral pathogenesis, cell-mediated immunity, and genetic resistance to infection. Following low dose inoculation of ECTV into the footpad, the disease course is delayed involving successive local and systemic spread mimicking smallpox though with more rapid severe disease onset and accentuated hepatic involvement. Aerosol and intranasal ECTV infection models for ECTV have also been described. In an intranasal mouse (BALB/c and C57BL/6) model, there is a 7-day disease lag period and a 10-day mean time of death. Early virus proliferation in the lungs is followed by spleen and liver infection reaching peak values at 8 days.

The natural or original host of VACV is unknown, though vaccine-derived virus is now feral in Brazil and India and occasionally infects humans. VACV has a wide host range in the laboratory, and mice that are genetically resistant to ECTV are still susceptible to VACV. For vaccine studies, mice are usually challenged with a pathogenic strain of VACV such as Western Reserve (WR) by the intranasal route, but relatively high doses (10⁴-10⁶ PFU) are required, leading to pneumonia and morbidity and death within 7 to 10 days. In young rabbits, both rabbitpox (RPXV), a variant of VACV, and VACV WR cause a rapidly lethal systemic disease with skin lesions and aerosol transmission when administered by a variety of routes.

CPXV is endemic in wild rodents in Europe and central Asia and can be transmitted to cats, pet rats, and other animals to humans. In immunocompetent humans with no history of skin disorders, CPXV causes a localized lesion. No human-to-human transmission has been reported, and laboratory workers can be protected by a smallpox (VACV) vaccination. Genetic analysis indicates at least two major CPXV groups and several subgroups of CPXV. CPXV has many more immune modulators than other OPXV including VARV. In mice, the 50% lethal intranasal dose of the Brighton strain is similar to that of VACV WR. A strain of CPXV isolated from an outbreak in a monkey colony was shown to be highly lethal at relatively low dose in marmosets and may be useful as an OPXV non-human primate model.

As replication-competent and highly attenuated live vaccines elicit diverse immune responses, multiple factors may contribute to protective immunity. Moreover, the dominant mode of protection may depend on the virus/animal model, type and route of vaccination and challenge, and the interval after vaccination. Several studies, particularly with ECTV, demonstrate that cytokines, macrophages, NK cells, CD8⁺ T cells, and antibodies are each important for protection and clearing of a primary footpad infection in mice. In an immunocompetent mouse, either CD8⁺ T cells or antibody can provide protection. However, the importance of interferon, CD4+, and CD8⁺ T cells for protection against secondary ECTV infections, which is more pertinent in a vaccine context, has been questioned based on the kinetics of the recall humoral and cellular immune responses and studies with immune deficient mice. Immunized mice genetically deficient in CD8⁺ T cells or effector function were resistant to secondary infection with ECTV, whereas mice lacking B cells, major histocompatibility complex (MHC) class II, and CD40, which are important for antibody production, were unprotected. Depletion of CD4⁺ T cells prior to challenge with ECTV, however, did not interfere with protection, whereas depletion of B cells rendered the mice susceptible. Thus, in this model, antibody is necessary and sufficient for protection. The relatively slow kinetics of disease onset after intranasal infection of mice with ECTV has allowed the testing of post exposure vaccination. Significant protection was obtained by intramuscular vaccination with either Lister or MVA vaccine at 2 to 3 days after intranasal challenge with ECTV. High dose intravenous administration of MVA is particularly effective for 3-day post-exposure immunization and induces strong innate and adaptive immune responses. Studies with genetically deficient mice indicated that NK cells, CD4⁺ T cells, CD8⁺ T cells, and antibodies were all important under the latter conditions.

For mice vaccinated with replication-deficient or replication competent VACV and challenged intranasally with pathogenic VACV, antibody and CD8⁺ T cells contribute to protection, though to different degrees depending on the experimental protocol. B-cell-deficient mice unable to generate antibodies as well as β2-microglobulin-deficient mice unable to express MHC class I molecules for a CD8⁺ T-cell response were protectively vaccinated. However, mice with decreased CD4⁺ T cells or MHC class II expression and double-knockout mice deficient in MHC class I- and II-restricted activities were poorly protected or unprotected. Similarly, skin-scratch vaccinated B-cell-deficient pMT and T-cell-depleted mice were protected against a respiratory challenge with VACV. Thus, CD8⁺ T cells and antibody provide overlapping modes of vaccine protection in these mouse models. In the VACV WR challenge model, post exposure vaccination is not effective, probably because of the very rapid kinetics of disease and death.

Monkeys depleted of B cells to prevent an antibody response following live vaccination were susceptible to intravenous MPXV challenge, whereas vaccinated animals depleted of CD4+ and CD8⁺ T cells prior to challenge were resistant, indicating the dominant role of antibody under these conditions.

Protection provided by immunization with proteins is usually attributed to antibody. Six MV and two EV proteins are known to be targets of neutralizing or comet-reducing antibodies and immunizations with four of these purified proteins have been shown to protect mice against lethal challenge with VACV. Two MV proteins, A27 and H3, have roles in virus attachment, whereas L1 is involved in membrane fusion and entry as described in a preceding section. Recombinant A27 and H3, made in Escherichia coli, protected mice against VACV challenge by intraperitoneal and intranasal routes, respectively. In another study, however, mice immunized with a soluble recombinant A27 made in insect cells were not protected against a subsequent intranasal VACV challenge, despite the production of neutralizing antibody. The third MV membrane protein tested, L1, was also made in insect cells and provided protection against a lethal intranasal VACV challenge. The remaining two proteins tested, A33 and B5, are components of the EV membrane. B5 and A33 made either in E. coli or as secreted forms in insect cells were protective in VACV intranasal and ECTV footpad infection mouse models.

Immunizations with single proteins can prevent death, but weight loss and other signs of disease still occurred under most conditions. Immunization with a combination of proteins, particularly at least one MV and one EV protein provides greater protection. The trivalent vaccine used in the latter study consisted of secreted forms of one MV protein (L1) and two EV proteins (A33 and B5) made in insect cells and mixed with adjuvant. This combination also provided protection against MPXV in monkeys, as did a quadravalent combination also containing the A27 MV protein with alum and CpG. Although the above animal model experiments were carried out using VACV proteins, there could be an advantage to using the corresponding VARV proteins if they were to be developed as vaccines.

Immunizations with DNA encoding OPXV proteins may induce antigen-specific CD8⁺ T cells in addition to antibodies. Conclusions regarding the synergistic effects of targeting MV and EV proteins were obtained using vaccines comprised of DNA encoding the MV proteins A27, L1, D8 and the EV proteins A33 and B5 in mouse and monkey studies. Partial protection was also obtained with DNA encoding the MV proteins A28 plus H2, but these were not tried in conjunction with DNA encoding EV proteins. Priming with a DNA vaccine consisting of programmed immune cells comprising: We produced in vitro-hematopoetic, mesenchymal, connective tissue, endothelial, cardio, induced pluripotent, osteo, muscle, and/or soft muscle tissue stem cells from a subject; expanding the stem cells from a culture; differentiating the stem cells and; exposing the differentiated stem cells to viral particle to generate immune response triggering surface antigen expression in the differentiated cells; and combining said cells so as to comprise a mixture of differentiated cells obtained from about 46% hematopoetic stem cells, about 35% mesenchymal stem cells, about 18% connective tissue, endothelial, cardio, induced pluripotent, osteo, muscle, and/or soft muscle tissue stem cells, and boosting with subunit proteins made in bacteria provided better protection in a MPXV model than either alone. Alphavirus replicons have also been used to deliver DNA. Additional vaccine candidates were obtained by a functional screening of a synthetic cowpox virus genome library in C57B1/6 mice.

Immune globulin from humans receiving smallpox vaccine (VIG) can provide protection when given to immune competent mice before or shortly after challenge with VACV or ECTV and prolong survival of SCID mice. Polyclonal antibodies to the L1 MV and B5R and A33 EV proteins protect against intranasal infection by VACV WR. Moreover, combinations of antibodies, either polyclonal or monoclonal, to MV and EV proteins provide superior protection than VIG or antibodies to single proteins or multiple EV proteins in immune competent or SCID mice. Interestingly, antibody to the L1 MV membrane protein or the A33 EV protein each provided protection against lethality but not weight loss when given post challenge, although one might have predicted that the latter would be superior because spread is mediated by EVs. Both human-like chimpanzee and fully human mAbs to MV (L1 or H3) and EV (A33 or B5) proteins also provide better protection than VIG in mouse models. In addition, mAbs could have safety advantages over VIG, which is made from pooled human sera. Although not permitted under present World Health Organization guidelines, the replacement of individual VACV or ECTV envelope proteins with VARV homologs might be useful for evaluating protection by mAbs in small animal models and, under appropriate containment conditions, safer than evaluating mAbs in VARV non-human primate models.

The studies have shown that peptides corresponding to CD8⁺ T-cell epitopes can fully protect mice in a lethal VACV intranasal or ECTV challenge model. In one large study, 49 different H-2b restricted epitopes were tested. The epitopes varied greatly in their ability to confer protection, with complete protection.

TABLE 5
Targets of neutralizing and protective antibodies

| | | | | Comet | |
|---|---|---|---|---|---|
| Protein | Location | Role | Neutralize | inhibit | Protection |
| A17 | MV | Assembly, | MV | − | NT^{a} |
| | | fusion | | | |
| A27 | MV | Attachment | MV | − | + |
| A28 | MV | Entry/fusion | MV | − | + |
| D8 | MV | Attachment | MV | − | + |
| H3 | MV | Attachment | MV | − | + |
| L1 | MV | Entry/fusion | MV | − | + |
| A33 | EV | Spread | — | + | + |
| B5 | EV | Spread | EV | + | + |

^{a}Not tested

### Example 44

Anthrax receptor (ATR) shares similarities with molecules relevant to hematopoiesis. This suggests that anthrax proteins might bind to these mimicking molecules and exert nonspecific hematopoietic effects. The hematopoietic system is the site of immune cell development in the adult. As such, ATR ligand, protective antigen (PA) and the other anthrax proteins, lethal factor (LF), edema factor (EF), could be significant to hematopoietic responses against Bacillus anthracis infection. Since hematopoiesis is the process of immune cell development, effects by anthrax proteins could be relevant to vaccine development. Here, we report on effects of anthrax proteins and toxins on early and late hematopoiesis. Flow cytometry shows binding of PA to hematopoietic cells. This binding might be partly specific since flow cytometry and western blots demonstrate the presence of ATR1 on hematopoietic cell subsets and the supporting PMI cells. Functional studies with long-term initiating cell (LTC-IC) and clonogenic assays determined hematopoietic suppression by anthrax toxins and stimulation by monomeric proteins. The suppressive effects were not attributed to cell death, but partly through the induction of hematopoietic suppressors, IL-10 and CCL3 (MIP-1a). In summary, anthrax proteins affect immune cell development by effects on hematopoiesis. The type of effect, stimulation or suppression, depend on if the stimulator is a toxin or monomeric protein. The studies show effects of anthrax proteins beginning at the early stage of hematopoieis, and also show secondary mediators such as IL-10 and CCL3. The roles of other cytokines and additional anthrax receptor are yet to be investigated.

Bacillus anthracis (B. anthracis) is a virulent spore-producing bacterium. The need to protect the civilian population was heightened by the anthrax mail attacks of 2001. The threat of bioterrorism and the potential use of biological weapons against both military and civilian populations have become a reality. Although immediate treatment could clear early anthrax infection, the subtle effects of an infection on the emerging immune system has not been examined. The use of B. anthracis as a lethal biological weapon has provoked renewed research interest. Although there is a rapid movement to develop effective vaccines against B. anthracis, there is no effective vaccine. The promise of an effective vaccine and host response rely on a functional emerging immune system, which encompass the early period of immune cell development up to the generation of mature immune cells. The complexity of anthrax toxins and the presence of its receptors in different cells provide challenges for vaccine development. This study addresses one area of the immune system, the early developmental process, namely hematopoietic system.

B. anthracis releases three monomeric proteins, lethal factor (LF), edema factor (EF) and protective antigen (PA). PA facilitates the biological activities of LF and EF by forming lethal toxin (LT) and edema toxin (ET), respectively. Once intracellular, PA is disassociated to release LT and ET where they mediate intracellular responses with pathophysiological effects, including septicemia.

The anthrax vaccine immunization program (AVIP), which was initially developed in the 1970s, uses a PA-based cell-free subunit, also known as “anthrax vaccine absorbed” (AVA). The AVA method requires multiple subcutaneous injections, mostly every two months up to 18 months, followed by annual boosts. The vaccine has been reported to exert local and systemic adverse reactions with contamination by ET and LT. The recognized need for large scale vaccine development and administration would not only require efficacy and safety, but also other scientific effects such as possible confound on the emerging immune system. It is possible that anthrax proteins in vaccines could have negative effects on the same cells and organs that are required to mediate protection against anthrax. The consensus for an improved vaccine has promoted interest in alternative strategies, such as live organism delivery systems, DNA vaccines, and purified recombinant PA.

Hematopoiesis is the process by which immune and other blood cells are produced from a finite number of hematopoietic stem cells (HSC). These stem cells reside in the bone marrow (BM) and are sources of life-long immune cell replacement. Insults to BM may affect all areas of the hematopoietic network, including effects on HSCs, their progenies, and other microenvironmental cells and structures. Thus, hematopoietic responses by anthrax would recapitulate the immune effects. In this regard, an understanding of the effects by anthrax proteins and/or toxins on hematopoiesis might provide insights on vaccine responses.

The BM is a complex organ with molecules that can bind to PA. Since PA is required for interactions with EF and LF, then the latter two molecules could interact specifically with hematopoietic cells that express ATR. At this time it is unclear if EF and LF could bind non-specifically to BM cells. Such information is important in light of a case study reporting on BM failure following anthrax vaccine administration. We sought to determine the effects of anthrax monomeric proteins and toxins on hematopoiesis. This central question has been addressed with healthy BM aspirates in an in vitro culture system in the presence or absence of purified anthrax proteins. The goal is to obtain insights into effects in the event of anthrax infection and expectations following vaccinations. The premise is that an understanding of anthrax toxins on the emerging immune system may lead to the prevention of organ damage by anthrax infection, and also aid in the development of interventional strategies for safe and effective vaccines.

The following were purchased from Sigma (St. Louis, Mo.): Polymyxin B, Ficoll-Hypaque, phosphate buffered saline, pH 7.4 (PBS), RNase A, Propodium Iodide, protein G Sepharose, α-MEM, Iscoves, DMEM with high glucose, RPMI 1640, bovine serum albumin (BSA), mouse anti-β-actin, non-immune murine IgG and non-immune rabbit IgG. Fetal calf sera (FCS) and horse sera were purchased from HyClone Laboratories (Logan, Utah). The following were purchased from R&D Systems (Minneapolis, Minn.): Recombinant human (rh) GM-CSF, rhEpo, SCF, IL-3 and anti-IL-10 mAb, IL-10 and CCL3. Rabbit polyclonal anti-CCL3 was generously provided by the Immunology Department of Genetics Institute (Cambridge, Mass.).

Antibodies that were ordered from BD Bioscience (San Jose, Calif.) follow: Purified mouse anti-human monoclonal antibody CD38; rabbit anti-human caspase-3; Phycoerythrin (PE) mouse anti-human CD34; PE-Cy5 mouse anti-human: CD117, CD7, CD41a, CD38, CD71; Allophycocyanin (APC) mouse anti-human: CD38, CD79a, CD32, CD45RA, CD235a; Peridinin chlorophyll protein (PerCP) mouse anti-human CD 19; FITC-mouse IgG isotype; PE mouse IgG isotype; APC mouse IgG2b isotype control; PerCP mouse IgG isotype. PE-Cy5 rat anti-human CD 127 and PE-Cy5 Rat IgG2a isotype was purchased from eBioscience (San Diego, Calif.). Rabbit anti-human ATR1 was purchased from Alpha Diagnostics, Inc. (San Antonio, Tex.). Mouse anti-human vWF and CD31 were purchased from DAKO (Carpinteria, Calif.).

All commercial sources of media contained low endotoxin levels. Buffers are prepared with water from a MilliQ system with a filter that eliminates endotoxin. The following reagents were obtained through the NIH AIDS Research and Reference Reagent Program, Division of AIDS, NIAID, NIH: Anthrax Protective Antigen, Lot #9819: Anthrax Edema Factor, Lot #9820: Anthrax Lethal Factor, Lot #9821. The factors were received as lyophilized and then reconstituted in 1.0 mL of BSA at 1 mg/mL, diluted in endotoxin-free distilled water. Upon reconstitution, aliquots of 1 μl were stored in siliconized tubes and then stored at −80° C. The total amount of endotoxin present within the highest concentration of each protein (8 ng) was based on the level of endotoxin units (EU) provided on the data sheet: 10⁻⁴ ng EU for PA; 1.3×10⁻⁴ ng EU for LF and 1.9×10 for EF. To ensure that the functions are not due to endotoxin, we have boiled the proteins at 1 μg/mL for 30 min immediately before each assay. The levels of endotoxin were determined in triplicates with samples before and after boiling, using the QCL-1000 chromogenic LAL endpoint assay kit (Lonza Inc, Allendale, N.J.). The levels differed by ±0.01.

In vitro-programmed immune cells (PMI) were produced comprising: hematopoetic, mesenchymal, connective tissue, endothelial, cardio, induced pluripotent, osteo, muscle, and/or soft muscle tissue stem cells from a subject; expanding the stem cells from a culture; differentiating the stem cells and; exposing the differentiated stem cells to anthrax particle to generate immune response triggering surface antigen expression in the differentiated cells; and combining said cells so as to comprise a mixture of differentiated cells obtained from about 46% hematopoetic stem cells, about 35% mesenchymal stem cells, about 18% connective tissue, endothelial, cardio, induced pluripotent, osteo, muscle, and/or soft muscle tissue stem cells. (PMI will reference stem cell cocktail from this point on).

Endothelial cell cultures were established with unfractionated BM aspirates in media prepared with Endothelial Cell EGM-2 system (Cambrex, East Rutherford, N.J.). Bone marrow mononuclear cells (BMNCs) were isolated by Ficoll-Hypaque density gradient and then placed in basal media, containing components of the SingleQuot Kit, which is part of the EGM-2 system. A purified population of BM endothelial cells was determined if flow cytometry showed >99% positive for CD31 and vWF. This generally occurs after three passages.

Direct labeling of PA to FITC was performed with EZ-Label FITC Protein Labeling Kit (Pierce Biotechnology, Rockford, Ill.). PA was diluted in borate buffer and then incubated for 1 h with FITC reagent at a ratio of 1:24. Excess fluorochrome was removed by dialysis in PBS for 1 h using the Slide-A-Lyzer MINI Dialysis Unit. The conjugated PA was titrated by flow cytometry with human endothelial cells. The concentration that showed >95% positive for PA was selected as optimum.

Whole BM aspirates were studied for FITC-PA binding in 4-color flow cytometry. Red blood cells were first lysed with 1×BD Lyse then followed by fixing of the cells with 2% paraformaldehyde. The combinations of fluorochrome-conjugated antibodies and the expected outcome for specific cell subset follow: Myeloid-lymphoid lineages, CD34⁺/CD117⁺/CD38⁺; Common lymphoid progenitors, CD34⁺/CD7⁺/CD38⁻; Megakaryocytes, CD34⁺/CD41a⁺/CD38⁺; B-cell precursor, CD34⁺/CD127⁺/intracellular CD79a⁺; Mature B-cells, CD34⁺/CD19⁺/CD38⁺. Early myeloid progenitors, CD34⁺/CD32^{-/lo}/CD38^{+/-}; Primitive stem cells were identified with CD34+/CD45RA−; Committed myeloid or lymphoid stem cells were identified with CD34+/CD45RA+; Early erythroid progenitors, CD34⁺/CD235a⁺; Mature erythroid progenitors, CD34⁺/CD71⁺/CD45RA⁻; Mature granulocyte/monocyte progenitors, CD34⁺/CD71⁻/CD45RA⁺. None of the aforementioned antibodies was conjugated to FITC since the fourth, PA, was conjugated to FITC. Non-specific binding was studied in parallel with isotype control IgG conjugated to the appropriate fluorochrome. Compensation tubes were set up in parallel to correct for possible overlap among the different fluorochromes.

BMNCs were isolated by Ficoll-Hypaque density gradient and then used to positively select CD34⁺ cells. Selection was done with Dynal CD34 Progenitor Cell Selection System (Dynal, Inc., Lake Success, N.Y.). BMNCs were incubated with Dynabeads M-450 CD34 for 30 min at 4° C. Cells were released from the magnetic particles by incubating for 45 min at room temperature with DETACHaBEAD.

The retrieved CD34⁺ cells were subdivided into two subsets based on the expression of CD38: CD34⁺/CD38⁺ vs. CD34⁺/CD38⁻. CD38⁺ cells were selected with CELLection Pan Mouse IgG Kit (Dynal, Inc.). CD34+ cells were resuspended at 10⁶/mL and then incubated with CD38 monoclonal antibody at 1 μg/mL or 10 min at 4° C. After this, cells were washed and then incubated with CELLection Pan Mouse IgG Dybnabeads for 20 min at 4° C. Cells were released from the beads by gentle rotation for 15 min at room temperature with DNase I Releasing Buffer. Immunofluorescence showed >90% cell purity.

Cell membrane extracts were obtained from CD34⁺/CD38⁻, CD34⁺/CD38⁺ and PMI cells as previously described. Cells were incubated with 400 μL of 1× lysis buffer (Promega, Madison Wis.) for 15 min at room temperature. Cell lysates were pelleted by centrifugation at 10,000 g for 15 min at 4° C. The pellets with membrane fractions were resuspended in 300 μL PBS and then vortex. The total protein concentration was determined by BioRad protein assay (Bio-Rad, Hercules, Calif.). Supernatants containing cytosolic fractions were similarly analyzed. Whole-cell extracts were obtained by four cycles of freezing and thawing in 400 μL 1× lysis buffer followed by centrifugation at 10,000 g for 15 min at 4° C. Supernatants containing the whole cell extracts were analyzed for total protein levels.

ATR was immunoprecipitated from cell membrane and cytosolic extracts obtained from CD34⁺/CD38⁻ and CD34⁺/CD38⁺ cells. Cell extracts were incubated with rabbit anti-human ATR (1/1,000) at 4° C. overnight. After this, samples were incubated with protein G Sepharose at 4° C. for 6 h. Reactions were centrifuged at 4° C., 10 000 g for 30 min. The pellets were washed once with PBS, resuspended in sample buffer, and electrophoresed on 12% SDS-PAGE. Proteins were transferred to PVDF membranes (Perkin Elmer, Wellesley, Mass.), and ATR was detected by overnight incubation at 4° C. with rabbit anti-human ATR at 1/1,000 dilution. After this, membranes were washed and incubated with horseradish peroxidase (HRP)-conjugated goat anti-rabbit IgG (1/2,000) for 1 h at 4° C. HRP was developed with chemiluminescence western blot detection reagents (Perkin Elmer, Wellesley, Mass.). The molecular weights were determined by comparing to Kaleidoscope prestained standards (BioRad, Hercules, Calif.). Although the total proteins per sample in the immunoprecipitation assay were similar, we nonetheless performed western blots in parallel for β-actin. Caspase-3 was not immunoprecipitated, but was detected by similar methods using rabbit anti-human caspase-3 at 1/1,000 dilution with BMNC whole cell extracts.

Cytokine production in BM stroma used two methods: 1) Microarray III (Ray Biotech, Norcross, Ga.), and 2) ELISA kits for MIP-1a and IL-10, purchased from R&D Systems. The array studies were analyzed according to the manufacturer's instructions, as previously described. Membranes were incubated for 30 min in 1× blocking buffer provided with the kit, followed by the addition of 1 mL sera-free conditioned media obtained from confluent stroma. The membranes were incubated at room temperature for 1 h then subjected to three 5-min washes with 1× Wash Buffer I, followed by two 5-min washes with 1× Wash Buffer II, also provided with the kit. The membranes were incubated for 1 h with biotin-conjugated antibodies and then developed for 1 h with HRP-conjugated streptavidin and detection reagent mix provided in the kit. ELISA for IL-10 and CCL3 were done with the same samples used for the array studies.

Short-term BM clonogenic assays were performed with BMNCs as previously described. Briefly, BMNCs (10⁵/mL) were assayed in the presence or absence of LF, EF, PA, or combinations, each at 1, 2, and 8 ng/mL. Control cultures contained vehicle. The assays were performed in methylcellulose matrices for CFU-GM and CFU-GEMM.

Cultures for CFU-GM contained 3 U/mL rhGM-CSF while CFU-GEMM assays contained 3 U rhGM-CSF, 3 U/mL rhIL-3, 10 ng/mL SCF, and 2 U/mL rhEpo. CFU-GM colonies were counted at day 8 by a blind observer (PR). Colonies with >20 cells were included. CFU-GEMM colonies were counted at day 21. Random colonies of CFU-GEMM were placed on slides and then stained by Wright's to verify mixed cell lineages.

LTC-IC cultures were performed as previously described. Confluent PMI cells were cultured in 25-cm² flasks and then subjected to 150 Gy, delivered by a cesium source. BMNCs were added to the PMI cultures at 10⁷ cells/flask. Cultures were performed in the presence or absence of anthrax proteins at 1 and 2 ng/mL. Each experimental point was performed in duplicate. One set was studied at week 5 for CFU-GEMM progenitors and the other at week 12.

TUNEL assays were performed with the DeadEnd Fluorometric TUNEL System (Promega), following manufacturer's recommendation for suspension cells. BMNCs were incubated for different times in the presence or absence of LF, EF, PA, added singly, or in combinations, each at 2 ng/mL. Positive controls were incubated overnight with 5 μg/mL cycloheximide and 10 ng/mL TNFa. For the analyses, BMNCs were resuspended in PBS at 2×10⁷ cells/mL. Aliquots of cells, 100 μL, were added to superfrost slides and then fixed with 0.4% paraformaldehyde for 25 min at 4° C. Slides were washed twice in PBS for 5 min at room temperature, permeabilized for 5 min and then incubated with Equilibration Buffer for 10 min. After this step, the DNA strand breaks were labeled with fluorescein-12-dUTP for 1 h at 37° C. in a humidified chamber. The reaction was terminated with 2×SSC for 15 min at room temperature. The samples were then washed thrice with PBS and mounted in VECTASHEILD+DAPI (300 nM) to stain the nuclei. The total number of TUNEL positive cells was determined by fluorescence microscopy with >200 cells were counted for each experiment.

BM cells were treated with 2 ng/mL EF, LF, PA or combinations for 4 h. After this, cells were washed, treated with RNase A (1 mg/mL) and then fixed in 70% ethanol. Cells were stained with 20 μg/mL Propodium Iodide. DNA analysis was done by FACScan (BD Biosciences) and the analysis was performed using CellQuest software (BD Biosciences).

Total RNA was extracted from BM mononuclear cells and 2 μg was reverse transcribed and then subjected to PCR with ATR1 primers, spanning+329/+569, 5′ cca gaa agt tct gcc agg ag 3′ (forward) and 5′ gcc agc tgt gtc tca ttg as 3′ (reverse). The cycling profile for ATR1 was 35 cycles with 95° C. for 45 secs, 55° C. for 30 secs, 72° C. for 1 min 30 secs and extension for 7 mins. The same cDNA was normalized with primers for 3-actin spanning+842/+1037, 5′ tgc cct gag gca ctc ttc 3′ (forward; SEQ ID NO: 10) and 5′ gtg cca ggg cag tga tct 3′ (reverse (SEQ ID NO: 11). The cycling profile for f-actin was 30 cycles, 95° C. for 45 secs, 55° C. for 30 secs, 72° C. for 1 min 30 sec and extension for 7 mins.

Statistical evaluations of the data were done with analysis of variance and Tukey-Kramer multiple comparisons test. A P value of <0.05 was considered significant.

The first set of studies determined whether anthrax toxin receptor 1 (ATR1) is expressed on two major BM cell subsets: CD34⁺/CD38⁻, which comprises mostly hematopoietic stem cells, HSCs and CD34⁺/CD38⁺ (mostly hematopoietic progenitors). ATR1 was selected because it is the uncleaved form of the three ATR subtypes. The cell subsets were isolated from freshly obtained BM aspirates from healthy donors. The low frequency of HSCs could compromise sensitivity. We therefore employed combinations of immunoprecipitation and western blots. Parallel western blots were performed for β-actin to ascertain equal amounts of proteins in the starting samples.

Analyses were performed with membrane extracts since cytoplasmic retention of ATR1 would indicate non-responsiveness to exogenous anthrax proteins. Membrane extracts from the two major cell subsets were subjected to combined immunoprecipitation/Western blots with anti-ATR1. Western blot analyses showed bands at molecular weights at the expected size of ATR1, ˜50-60 kDa, for both cells subsets. Each lane was studied with extracts from a different donor. The detection could not be due to cytosolic contamination since there was undetectable band in lanes with cytosolic extracts.

Computer analyses showed similarities among ATR1 and hematopoietic-relevant molecules. Thus, it is possible that PA could bind to different subsets of hematopoietic cells, through interactions with molecules other than ATR1. This question was addressed in 4-color flow cytometry with fluorochrome labeled PA and antibodies specific for different hematopoietic cell subsets. Antibodies to CD34, CD117 (c-kit), CD32 (FcR) and CD38 were used to differentiate between myelo-lymphoid progenitors (CD34⁺/CD117⁺/CD38^{lo/-}) and early myeloid progenitors (CD34⁺/CD32^{lo}/CD38^{+/-}). The CD34⁺/PA⁺ subset showed 46±6% dim/low fluorescence for CD38, and negative to dim for the myeloid marker, CD32. This indicates binding of PA to early myeloid progenitors. It was further determined if CD34⁺/PA⁺ cells comprised CD117 (c-kit), which is expressed on the most primitive hematopoietic cells. As expected, most CD38⁺ cells were negative for CD117, verifying a mature hematopoietic phenotype. A small subset of immature hematopoietic cells that bind PA was detected, 1.4±0.01% CD38⁺/CD117⁺. The results described in this section show PA interaction with both immature and mature CD34⁺ hematopoietic cells.

Western blots and immunofluorescence indicated expressions of ATR1, and PA-binding to both primitive and mature hematopoietic progenitors. It was therefore asked whether PA binding is significant to hematopoietic functions. First mature progenitors of granulocyte-monocyte lineage (CFU-GM) were examined in short-term clonogenic assays. The assays were performed with BM mononuclear cells (BMNCs) from seven different healthy donors, in the presence or absence of monomeric LF, EF and PA at 1, 2 or 8 ng/mL. Dose-response curves were performed with proteins ranging between 10⁻⁴ and 10³ ng/mL. For each protein, the effects were similar between 1 and 8 ng/mL. Studies with combined proteins contained equal amounts of each. This ratio differed from other studies that added 5× more LF and EF to PA. This difference might be attributed to differences in the experimental systems.

The hematopoietic response of each protein is concentration-dependent. Compared to baseline colonies (media alone), LF caused no significant (p>0.05) proliferation at 1 and 2 ng/mL, but significance (p<0.05) at 8 ng/mL. In the case of EF, we observed a bell-shaped effect with significant increase at 2 ng/mL. Cultures with PA showed dose-dependent suppression on CFU-GM, with significant (p<0.05) increases at 1 and 2 ng/mL, and significant (p<0.05) suppression at 8 ng/mL. In summary, each monomeric anthrax toxin showed a specific effect on CFU-GM.

It was next verified that the effects of anthrax proteins are not due to endotoxin. This question was addressed by repeating the assays with the denatured proteins, which was achieved by boiling for 20 mins. The results showed no significant (p>0.05) change in CFU-GM, indicating that the outcome cannot be explained by endotoxin.

Anthrax toxins are formed when LF and/or EF binds to PA with subsequent endocytosis of heptamers and the releases of LF and LT. The CFU-GM cultures were therefore repeated with combinations of anthrax proteins. There was significant (p>0.05) increases in CFU-GM for LF+EF, but not for EF+PA or LF+PA. In the presence of EF+LF+PA, there were significant (p<0.05) suppression. In summary, this section shows varied effect of combined proteins. However, anthrax toxin caused suppression on CFU-GM.

Western blots and flow cytometry studies indicate that primitive hematopoietic progenitors express ATR1 and can also bind PA. We therefore studied the effects of proteins at 1, 2 and 8 ng/mL on primitive hematopoietic progenitors using long-term culture initiating cell (LTC-IC) assays. An LTC-IC cell is indicated if the cell can generate mixed progenitors along granulocytic, erythrocytic and megarkaryocytic lineages (CFU-GEMM). We selected 2 ng/mL of anthrax toxin (combinations of LF, EF and PA) for the LTC-IC assay. We observed suppression on LTC-IC for both 6- and 12-wk cultures. Wk 12 outcome has physiological significance since this time period recapitulates the responses expected of early hematopoietic progenitors that are close to the maturation of HSCs. There was significant (p<0.05) reduction in CFU-GEMM for monomeric and combined proteins at both 6- and 12-wk cultures. In summary, this section shows suppressive effects of anthrax proteins on CFU-GM and CFU-GEMM progenitors.

Despite lack of evidence for endotoxin in the anthrax protein samples, we verified that the effects on LTC-IC were not due to endotoxin. LTC-IC studies were repeated with boiled samples and the results were similar to cultures with media alone. Similarly, when the assay was repeated in the presence of the endotoxin inhibitor, polymyxin B (12.5 μg/mL), the results were similar to cultures with anthrax proteins alone, ±0.02, further indicating that the effects by anthrax proteins on LTC-IC are not due to endotoxin.

The section describes studies to determine whether the suppressive effects of anthrax proteins could be explained by cell death. Firstly, BM mononuclear cells (BMNCs) were incubated with or without anthrax toxins. In one set of experiment, the media were not changed. In parallel experiment, there were biweekly media changes with fresh anthrax proteins. In both cases, trypan blue exclusion indicated <5% cell death by wk 12. Flow cytometry for the total number of CD34+ cells showed no change by wk 12. Since there was no Further verification was done with TUNEL assay and Western blot for active caspase-3. BMNCs from 7 different donors were incubated with various combinations of anthrax proteins, each at 2 or 8 ng/mL. At wks 1, 2, 4, 8 and 12, the cells were analyzed for percentages of TUNEL positive cells. Baseline cultures (no anthrax proteins) showed 2±0.55%, n=7. Since the total TUNEL positive cells were similar for all time periods, the data are shown for 12-wk cultures. Except for studies with EF, LF and PA, the total TUNEL positive cells were not significantly different from baseline studies. Positive controls performed with cycloheximide and TNFα showed approximately 50% TUNEL staining. Western blots for caspase-3 showed undetectable bands. Together, the data indicate that the suppressive effects of anthrax proteins and toxins on hematopoiesis cannot be explained by cell death. Since B anthracis and other bacterial agents have been linked to macrophage death involving caspase 1, this mediator might be involved as a secondary mechanism in macrophages. However, there is no evidence of its involvement in the CD34⁺ cell population. Caspase 1 has been shown to precede caspase 3 in apoptosis of hematopoietic cells. In this case, we have not detected caspase 3, suggesting that caspase 1 might not be involved, at least for hematopoietic progenitors.

This section describes studies to determine whether anthrax toxins can affect the major cellular support of hematopoiesis, stroma. To address this question, we focused on cytokine production because of their major roles in hematopoiesis. We first determine whether BM stroma express ATR1. Immunoprecipitation/western blots with membrane extracts from stroma of four different healthy donors showed single dense bands. Since ATR1 is undetectable in cytoplasmic extracts of CD34⁺/CD38⁺ cells, this served as negative control. Parallel western blots were done for β-actin for the purpose of normalization.

It was next asked if anthrax toxin can induce the production of cytokines and chemokines by protein arrays. PMI cells were incubated with LF+EF+PA, each at 2 ng/mL. After 16 h, the analyses were done with culture media and whole cell extracts. The results are shown for normalized densities of two hematopoietic suppressors, IL-10 and MIP-1a (CCL3). In both cases, there were significant (p<0.05) increases in IL-10 and CCL3 by anthrax toxins as compared to unstimulated stroma. We also observed a 2-fold increase for SDF-1α (CXCL12) production with LF+EF+PA, each at 2 ng/mL. This increase is intriguing since CXCL12 could cause the release of HSCs from their niche within BM. IL-10 and MIP-1α levels were quantitated by ELISA and the values were placed at the top of the bar graphs of the array results.

CCL3 and IL-10 can mediate hematopoietic suppression. Since there is no compelling evidence of cell death by anthrax proteins, we asked whether hematopoietic suppression could be partly explained by secondary production of IL-10 and CCL3. LTC-IC assays were performed in the presence or absence of LF+EF+PA, each at 2 ng/mL (toxin), in the presence or absence of anti-IL-10 and/or anti-CCL3, each ranging between 1 and 1000 ng/mL. Control cultures contained similar concentrations of non-immune species IgG. CFU-GEMM colonies, the endpoints of the LTC-IC assays, were similar between 5 and 100 ng/mL of antibodies. We have determined specificity of the antibodies since non-immune IgG showed total CFU-GEMM similar to cultures with media alone. While neither antibody was able to fully reverse the suppressive effects of toxins, the degree of reversal was significant (p<0.05) as compared to cultures with toxins alone.

Positive controls were established with media (baseline), in the presence of 50 μg/mL each, IL-10 and CCL3. Since these are not the only hematopoietic suppressors, their combined effects, although significantly suppressive, did not totally blunt the formation of CFU-GEMM. The concentrations of IL-10 and CCL3 were selected based on the total levels induced by the anthrax proteins. In addition, IL-10 and CCL3 with 5 ng/mL of the respective antibody blunted the ability of these two factors to exert hematopoietic suppression to revert to levels observed in cultures with media alone. These studies supported the neutralization effects of the antibodies. In summary, the results show CCL3 and IL-10 as partial mediators in anthrax-induced suppression on LTC-IC cells.

Differences were observed in BM functions depending on the combinations of anthrax toxins. Cytokine analyses also correlated with the suppressive effects of anthrax proteins on hematopoiesis. BM cells with anthrax proteins were studied to determine if the suppressive effects could be explained by blocking of specific regions of the cell cycle. To this end, whole BM cells were incubated with 2 ng/mL of single or combined anthrax proteins. After 4 h, the cells were analyzed by FACScan. The results showed significant changes for cells incubated with the 3 proteins. Since the incubation period was relatively short, the results suggest that anthrax proteins might rapidly affect hematopoietic cell cycle when by three combinations that are required to comprise a toxin.

This study reports on hematopoietic alterations by anthrax proteins and toxins, which could occur directly and/or indirectly through the productions of cytokines and chemokines. Hematopoietic suppression was focused on as significant effects were observed for the most primitive hematopoietic progenitors, which were based on LTC-IC assays. The most interesting observation of this study is the fact that anthrax toxins show a non-canonical effect as opposed to the classical report of toxins formed by EF, LF and PA. The binding of PA to the two subsets of CD34⁺ indicate that there is the potential for direct effects on hematopoiesis. In addition, the effects on hematopoiesis could occur through molecular mimicry with PA interacting with other homologous molecules that are relevant to hematopoiesis. The alignment of homologous molecules with relevance to hematopoiesis, although not addressed in functional assays might explain the direct effects of PA. These are exciting observations. Current studies are underway to knockdown ATR1 and/or CMG2 to determine if PA binds non-specifically to the homologous molecules.

Of interest are the functional differences between 1 and 2 ng/mL of toxins. These differences were interpreted to indicate that the cells are sensitive to small changes in the levels of anthrax toxins. Future plans are in place to explore these small changes because the hematopoietic system could be sensitive to vaccine administration and also, to low levels of anthrax exposure. The latter could be a scenario if the individuals have competent immune systems, or in cases where there is rapid responses to antibiotics. Another interesting observation is that conversion of anthrax proteins to molar ratio did not follow the expected optimal effects by ratios of 2:1. This further verifies that the effects of anthrax proteins on hematopoiesis is non-canonical and require in-depth studies.

The effects of anthrax proteins and toxins are different on the most primitive and the more mature hematopoietic progenitors. These differences are shown in the studies of myeloid progenitors and cells close to the maturational stage of hematopoietic stem cells. The partial subunits with EF or LF and PA show minimal inhibition as compared to significant inhibition in studies with the entire complex of EF, LF and PA. This suggests that the complex could determine the outcome that might be direct or indirectly mediated through other non-progenitor cells. The interesting observation is the stimulator effect of LF and EF. Regardless of the mechanism, it is evident that these factors show varying effects on progenitors and that the outcome depends on small changes in each and combined toxins.

Anthrax toxins showed significant suppression on LTC-IC cells. Since these cells represent the most primitive hematopoietic cells, in particular the 12-week cultures, it is possible that the toxins could be protective and prevent cycling, or reduce the total numbers of these primitive cells. In the case of the latter, the toxins could lead to BM ablation and exposures to the toxins could cause possible hematopoietic failure.

While two of the identified factors, IL-10 and CCL3, have been shown to have secondary effects on LTC-IC cells, other mechanisms appear to be operative. This premise is based on studies that show failure to totally reverse the suppressive effects of anthrax toxins despite cytokine neutralization. Ongoing studies are designed to fully understand the mechanisms by which anthrax toxins mediate hematopoietic suppression through cytokine and/or chemokine production. Also, ongoing studies are directed towards an understanding of the intracellular changes caused by anthrax toxins. An unanswered question is to identify the effects of the toxins on candidate subsets within the myelo-lymphoid lineages. These studies, combined with ongoing and future studies would provide detailed insights on the mechanisms by which anthrax infection and vaccines affect adult hematopoiesis.

Although we have examined the effects of anthrax toxin on the emerging immune system, namely hematopoiesis, the toxins have effects on mature cells. In particular B-cells, whose proliferation and IgG production are inhibited by LT. While lethal toxins inhibit MAP kinase activity, this role on anthrax-mediated effects on hematopoiesis is unclear. Septic shock, caused by anthrax infections cannot be explained by cytokine production as causative agents. This underscore the need for dissected studies, such as the case presented in this report to understand the effects of anthrax proteins and toxins on hematopoiesis and other biological functions.

The results of 6-wk LTC-IC cultures represent the more mature progenitors. Anti-MIP-1a resulted in more pronounced reversal of CFU-GEMM in the 6-wk cultures as compared to the 12-wk assays. This suggests that CCL3 is important for the mature progenitors rather than the immature progenitors. IL-10 effects were similar for both the 6- and 12-wk cultures suggesting that this cytokine could be acting at all levels. The biology of IL-10 with respect to hematopoietic effects is complex. In some cases, IL-10 could be stimulatory to hematopoiesis since it suppresses the activity of IFNγ, which could inhibit hematopoiesis at high levels. The fact that neither anti-CCL3 nor anti-IL-10 was able to reverse the hematopoietic effects of the anthrax toxin indicates that other mechanisms are important. Future studies are underway to address these mechanistic processes.

Although we could not detect evidence of cell death by single or combined anthrax proteins, prolonged exposures in 12-wk cultures to anthrax toxin (EF/LF/PA) showed a trend towards apoptosis, as compared to baseline cultures. This finding could be important and might contribute to hematopoietic failure during infection. The question is whether particular subset of BM cells might be sensitive to prolonged anthrax exposures. In addition, there was no evidence of cell death in the LTC-IC cultures, suggesting that the most primitive hematopoietic cells could not account for apoptosis. Cell cycle analyses with quite remarkable. It is interesting that combination of two toxins show increases in cells in the S as compared to the combinations of the toxins EF/PA/LF where the cells show significant reduction in cells in the S-phase. These results are consistent with reduced clonogenicity in the presence of combined toxins. The cell cycle analyses were performed during 4-h incubation to determine the relatively early response by BM cells. It is yet to be determined what chronic exposure of the anthrax toxin would show.

A recent report showed inhibitory effects on B-cell proliferation and immunoglobulin production. This indicates that the anthrax toxins are affecting both the hematopoietic primitive progenitors, and the mature B-cells. While the other hematopoietic lineages have not been examined, these questions are important and are the subject of ongoing studies. The report advises that current and future anthrax vaccine development should consider BM dysfunction. This dysregulation, especially in the hematopoietic system, may lead to repercussions in the body's ability to respond to anthrax infection as well as the ability to maintain vaccine-based protection. It is understandable that in the current state of increased biological weapons threat, the quick production of an effective anthrax vaccine is favored. However, certain concerns must be addressed to prevent potential harm to the general population if given the anthrax vaccine. In addition, vaccine development for other biothreat agents may benefit from similar consideration.

In conclusion, this study shows hematopoietic effects by anthrax proteins and toxins. These effects could be directly mediated by ATR1 present on different hematopoietic cell subsets, including the most primitive type of cells. The effects could also be indirect through PMI cells. Specifically, this could occur by the toxins inducing cytokine production to modulate hematopoiesis. Anthrax toxin could also target actin filaments, destruction of which could lead to cellular instability. The observed effects by EF and LF, in the absence of PA are of significance. It appears that these factors could be interacting with other molecules found in BM. These are intriguing findings and form the basis for current studies in the laboratory. These studies could lead to an understanding of health disorders associated with anthrax exposures, and could also be explored for efficient responses by BM to anthrax vaccines.

Each of the non-limiting examples can stand on its own, or can be combined in any permutation or combination with any one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions. Embodiments defined by each of these transition terms are within the scope of this invention.

Moreover, in the following claims, the terms “first,” “second,” and “third,” etc. are used merely as labels, and are not intended to impose numerical requirements on their objects.

All numerical designations, e.g., pH, temperature, time, concentration, and molecular weight, including ranges, are approximations which are varied (+) or (−) by increments of 0.1. It is to be understood, although not always explicitly stated, that the reagents described herein are merely exemplary and that equivalents of such are known in the art.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

Unless otherwise indicated, conventional techniques of molecular biology (including recombinant techniques), microbiology, cell biology, biochemistry and immunology were employed, which are within the skill of the art. Such techniques are explained fully in the literature. These methods are described in the following publications. See, e.g., Sambrook et al. MOLECULAR CLONING: A LABORATORY MANUAL, 2nd edition (1989); CURRENT PROTOCOLS IN MOLECULAR BIOLOGY (Ausubel et al. eds. (1987)); the series METHODS IN ENZYMOLOGY (Academic Press, Inc.); PCR: A PRACTICAL APPROACH (M. MacPherson et al. IRL Press at Oxford University Press (1991)); PCR 2: A PRACTICAL APPROACH (MacPherson, Hames and Taylor eds. (1995)); ANTIBODIES, A LABORATORY MANUAL (Harlow and Lane eds. (1988)); USING ANTIBODIES, A LABORATORY MANUAL (Harlow and Lane eds. (1999)); and ANIMAL CELL CULTURE (Freshney ed. (1987)).
WO 2013127976
1. 1. Feldmann, H and Geisbert, T W (2011). Ebola haemorrhagic fever. Lancet 377: 849-862.
2. MacNeil, A. and Rollin, P E (2012). Ebola and Marburg hemorrhagic fevers:
neglected tropical diseases? PLoS Negl Trop Dis 6: e1546.
3. Borio, L, Inglesby, T, Peters, C J, Schmaljohn, A L, Hughes, J M, Jahrling, P B et al.; Working Group on Civilian Biodefense. (2002). Hemorrhagic fever viruses as biological weapons: medical and public health management. JAMA 287: 2391-2405.
4. Warfield, K L and Olinger, G G (2011). Protective role of cytotoxic T lymphocytes in filovirus hemorrhagic fever. J Biomed Biotechnol 2011: 984241.
5. Wauquier, N, Becquart, P, Padilla, C, Baize, S and Leroy, E M (2010). Human fatal zaire ebola virus infection is associated with an aberrant innate immunity and with massive lymphocyte apoptosis. PLoS Negl Trop Dis 4: e837.
6. Falzarano, D, Geisbert, T W and Feldmann, H (2011). Progress in filovirus vaccine development: evaluating the potential for clinical use. Expert Rev Vaccines 10: 63-77.
7. Warfield, K L, Olinger, G, Deal, E M, Swenson, D L, Bailey, M, Negley, D L et al. (2005). Induction of humoral and CD8+ T cell responses are required for protection against lethal Ebola virus infection. J Immunol 175: 1184-1191. I
8. Grant-Klein, R J, Altamura, L A and Schmaljohn, C S (2011). Progress in recombinant DNA-derived vaccines for Lassa virus and filoviruses. Virus Res 162: 148-161.
9. Kalina, W V, Warfield, K L, Olinger, G G and Bavari, S (2009). Discovery of common marburgvirus protective epitopes in a BALB/c mouse model. Virol J 6: 132.
10. Olinger, G G, Bailey, M A, Dye, J M, Bakken, R, Kuehne, A, Kondig, J et al. (2005). Protective cytotoxic T-cell responses induced by venezuelan equine encephalitis virus replicons expressing Ebola virus proteins. J Virol 79: 14189-14196.
11. Sullivan, N J, Hensley, L, Asiedu, C, Geisbert, T W, Stanley, D, Johnson, J et al. (2011). CD8⁺ cellular immunity mediates rAd5 vaccine protection against Ebola virus infection of nonhuman primates. Nat Med 17: 1128-1131.
12. Geisbert, T W, Bailey, M, Geisbert, J B, Asiedu, C, Roederer, M, Grazia-Pau, M et al. (2010). Vector choice determines immunogenicity and potency of genetic vaccines against Angola Marburg virus in nonhuman primates. J Virol 84: 10386-10394.
13. Fenimore, P W, Muhammad, M A, Fischer, W M, Foley, B T, Bakken, R R, Thurmond, J R et al. (2012). Designing and testing broadly-protective filoviral vaccines optimized for cytotoxic T-lymphocyte epitope coverage. PLoS ONE 7: e44769.
14. Hensley, L E, Mulangu, S, Asiedu, C, Johnson, J, Honko, A N, Stanley, D et al. (2010). Demonstration of cross-protective vaccine immunity against an emerging pathogenic Ebolavirus Species. PLoS Pathog 6: e1000904.
15. Zahn, R, Gillisen, G, Roos, A, Koning, M, van der Helm, E, Spek, D et al. (2012). Ad35 and ad26 vaccine vectors induce potent and cross-reactive antibody and T-cell responses to multiple filovirus species. PLoS ONE 7: e44115.
16. Geisbert, T W and Feldmann, H (2011). Recombinant vesicular stomatitis virus-based vaccines against Ebola and Marburg virus infections. J Infect Dis 204 Suppl 3: S1075-S1081.
17. Grant-Klein, R J, Van Deusen, N M, Badger, C V, Hannaman, D, Dupuy, L C and Schmaljohn, C S (2012). A multiagent filovirus DNA vaccine delivered by intramuscular electroporation completely protects mice from ebola and Marburg virus challenge. Hum Vaccin Immunother 8: 1703-1706.
18. Bagarazzi, M L, Yan, J, Morrow, M P, Shen, X, Parker, R L, Lee, J C et al. (2012). Immunotherapy against HPV16/18 generates potent TH1 and cytotoxic cellular immune responses. Sci Transl Med 4: 155ra138.
19. Kee, S T, Gehl, J, Lee, E W (2011). Clinical Aspects of Electroporation. Springer: New York, N.Y.
20. Hirao, L A, Draghia-Akli, R, Prigge, J T, Yang, M, Satishchandran, A, Wu, L et al. (2011). Multivalent smallpox DNA vaccine delivered by intradermal electroporation drives protective immunity in nonhuman primates against lethal monkeypox challenge. J Infect Dis 203: 95-102.
21. Shedlock, D J, Talbott, K T, Wu, S J, Wilson, C M, Muthumani, K, Boyer, J D et al. (2012). Vaccination with synthetic constructs expressing cytomegalovirus immunogens is highly T cell immunogenic in mice. Hum Vaccin Immunother 8: 1668-1681.
22. Towner, J S, Khristova, M L, Sealy, T K, Vincent, M J, Erickson, B R, Bawiec, D A et al. (2006). Marburgvirus genomics and association with a large hemorrhagic fever outbreak in Angola. J Virol 80: 6497-6516.
23. Francica, J R, Varela-Rohena, A, Medvec, A, Plesa, G, Riley, J L and Bates, P (2010). Steric shielding of surface epitopes and impaired immune recognition induced by the ebola virus glycoprotein. PLoS Pathog 6: e1001098.
24. Hersperger, A R, Martin, J N, Shin, L Y, Sheth, P M, Kovacs, C M, Cosma, G L et al. (2011). Increased HIV-specific CD8+ T-cell cytotoxic potential in HIV elite controllers is associated with T-bet expression. Blood 117: 3799-3808.
25. Sullivan, N J, Sanchez, A, Rollin, P E, Yang, Z Y and Nabel, G J (2000). Development of a preventive vaccine for Ebola virus infection in primates. Nature 408: 605-609.
26. Dowling, W, Thompson, E, Badger, C, Mellquist, J L, Garrison, A R, Smith, J M et al. (2007). Influences of glycosylation on antigenicity, immunogenicity, and protective efficacy of ebola virus GP DNA vaccines. J Virol 81: 1821-1837.
27. Vanderzanden, L, Bray, M, Fuller, D, Roberts, T, Custer, D, Spik, K et al. (1998). DNA vaccines expressing either the GP or NP genes of Ebola virus protect mice from lethal challenge. Virology 246: 134-144.
28. Riemenschneider, J, Garrison, A, Geisbert, J, Jahrling, P, Hevey, M, Negley, D et al. (2003). Comparison of individual and combination DNA vaccines for B. anthracis, Ebola virus, Marburg virus and Venezuelan equine encephalitis virus. Vaccine 21: 4071-4080.
29. Swenson, D L, Warfield, K L, Negley, D L, Schmaljohn, A, Aman, M J and Bavari, S (2005). Virus-like particles exhibit potential as a pan-filovirus vaccine for both Ebola and Marburg viral infections. Vaccine 23: 3033-3042.
30. Kobinger, G P, Feldmann, H, Zhi, Y, Schumer, G, Gao, G, Feldmann, F et al. (2006). Chimpanzee adenovirus vaccine protects against Zaire Ebola virus. Virology 346: 394-401.
31. Jones, S M, Stroher, U, Fernando, L, Qiu, X, Alimonti, J, Melito, P et al. (2007). Assessment of a vesicular stomatitis virus-based vaccine by use of the mouse model of Ebola virus hemorrhagic fever. J Infect Dis 196 Suppl 2: S404-S412.
32. Dowling, W, Thompson, E, Badger, C, Mellquist, J L, Garrison, A R, Smith, J M et al. (2007). Influences of glycosylation on antigenicity, immunogenicity, and protective efficacy of ebola virus G P DNA vaccines. J Virol 81: 1821-1837.
33. Shedlock, D J, Bailey, M A, Popernack, P M, Cunningham, J M, Burton, D R and Sullivan, N J (2010). Antibody-mediated neutralization of Ebola virus can occur by two distinct mechanisms. Virology 401: 228-235.
34. Sullivan, N J, Geisbert, T W, Geisbert, J B, Shedlock, D J, Xu, L, Lamoreaux, L et al. (2006). Immune protection of nonhuman primates against Ebola virus with single low-dose adenovirus vectors encoding modified GPs. PLoS Med 3: e177.
35. Xu, L, Sanchez, A, Yang, Z, Zaki, S R, Nabel, E G, Nichol, S T et al. (1998). Immunization for Ebola virus infection. Nat Med 4: 37-42.
36. Warfield, K L, Swenson, D L, Olinger, G G, Kalina, W V, Aman, M J and Bavari, S (2007). Ebola virus-like particle-based vaccine protects nonhuman primates against lethal Ebola virus challenge. J Infect Dis 196 Suppl 2: S430-S437.
37. Sun, Y, Carrion, R Jr, Ye, L, Wen, Z, Ro, Y T, Brasky, K et al. (2009). Protection against lethal challenge by Ebola virus-like particles produced in insect cells. Virology 383: 12-21.
38. Choi, J H, Schafer, S C, Zhang, L, Kobinger, G P, Juelich, T, Freiberg, A N et al. (2012). A single sublingual dose of an adenovirus-based vaccine protects against lethal Ebola challenge in mice and guinea pigs. Mol Pharm 9: 156-167.
39. Richardson, J S, Yao, M K, Tran, K N, Croyle, M A, Strong, J E, Feldmann, H et al. (2009). Enhanced protection against Ebola virus mediated by an improved adenovirus-based vaccine. PLoS ONE 4: e5308.
40. Blaney, J E, Wirblich, C, Papaneri, A B, Johnson, R F, Myers, C J, Juelich, T L et al. (2011). Inactivated or live-attenuated bivalent vaccines that confer protection against rabies and Ebola viruses. J Virol 85: 10605-10616. Ebert, S, Lemmermann, N A, Thomas, D, Renzaho, A, Reddehase, M J and Holtappels, R (2012). Immune control in the absence of immunodominant epitopes: implications for immunotherapy of cytomegalovirus infection with antiviral CD8 T cells. Med Microbiol Immunol 201: 541-550.
41. Ruckwardt, T J, Luongo, C, Malloy, A M, Liu, J, Chen, M, Collins, P L et al. (2010). Responses against a subdominant CD8+ T cell epitope protect against immunopathology caused by a dominant epitope. J Immunol 185: 4673-4680.
42. Laddy, D J, Yan, J, Kutzler, M, Kobasa, D, Kobinger, G P, Khan, A S et al. (2008). Heterosubtypic protection against pathogenic human and avian influenza viruses via in vivo electroporation of synthetic consensus DNA antigens. PLoS ONE 3: e2517.
43. Yan, J, Yoon, H, Kumar, S, Ramanathan, M P, Corbitt, N, Kutzler, M et al. (2007). Enhanced cellular immune responses elicited by an engineered HIV-1 subtype B consensus-based envelope DNA vaccine. Mol Ther 15: 411-421.
44. Shedlock, D J, Talbott, K T, Wu, S J, Wilson, C M, Muthumani, K, Boyer, J D et al. (2012). Vaccination with synthetic constructs expressing cytomegalovirus immunogens is highly T cell immunogenic in mice. Hum Vaccin Immunother 8: 1668-1681.
45. Qiu, X, Alimonti, J B, Melito, P L, Fernando, L, Ströher, U and Jones, S M (2011). Characterization of Zaire ebolavirus glycoprotein-specific monoclonal antibodies. Clin Immunol 141: 218-227.
46. Richardson, J S, Abou, M C, Tran, K N, Kumar, A, Sahai, B M and Kobinger, G P (2011). Impact of systemic or mucosal immunity to adenovirus on Ad-based Ebola virus vaccine efficacy in guinea pigs. J Infect Dis 204 Suppl 3: S1032-S1042.
47. Warfield, K L, Posten, N A, Swenson, D L, Olinger, G G, Esposito, D, Gillette, W K et al. (2007). Filovirus-like particles produced in insect cells: immunogenicity and protection in rodents. J Infect Dis 196 Suppl 2: S421-S429.
48. Rao, M, Bray, M, Alving, C R, Jahrling, P and Matyas, G R (2002). Induction of immune responses in mice and monkeys to Ebola virus after immunization with liposome-encapsulated irradiated Ebola virus: protection in mice requires CD4(+) T cells. J Virol 76: 9176-9185.
49. Rao, M, Matyas, G R, Grieder, F, Anderson, K, Jahrling, P B and Alving, C R (1999). Cytotoxic T lymphocytes to Ebola Zaire virus are induced in mice by immunization with liposomes containing lipid A. Vaccine 17: 2991-2998.

All referenced publications, patents and patent documents are intended to be incorporated by reference, as though individually incorporated by reference.

What is claimed is:

1. A method to produce in vitro programmed immune stimulating cells comprising:
a) obtaining adipose cells from a subject;
b) expanding said cells from step a) in culture so as to yield one or more populations of stem cells;
c) differentiating said stem cells from step b) into hematopoetic stem cells, mesenchymal stem cells, connective cells, endothelial cells, cardio cells, osteo cells, muscle, cells and/or soft muscle tissue cells; and
d) exposing said differentiated cells from step c) to 3D/4D printed molds of preselected bacterial, virus or particles thereof or exposing said differentiated cells from step c) to bacterial, virus or particles thereof so as to generate immune response triggering surface antigen expression in said differentiated cells from step c), thereby yielding in vitro programmed immune stimulating cells.

2. The method of claim 1, wherein the subject is a human.

3. The method of claim 1, wherein the virus is selected from the group consisting of: adenoviruses; papillomaviruses; hepadnaviruses; parvoviruses; pox viruses; Epstein-Barr virus; cytomegalovirus (CMV); herpes simplex viruses; roseolovirus; varicella zoster virus; filoviruses; paramyxoviruses; orthomyxoviruses; rhabdoviruses; arenaviruses; coronaviruses; human enteroviruses; hepatitis A virus; human rhinoviruses; polio virus; retroviruses; rotaviruses; flaviviruses; hepaciviruses; and rubella virus.

4. The method of claim 1, wherein the bacteria is selected from the group consisting of: Bacillus; Bordetella; Borrelia; Brucella; Burkholderia; Campylobacter; Chlamydia, Chlamydophila; Clostridium; Corynebacterium; Enterococcus; Escherichia; Francisella; Haemophilus; Helicobacter, Legionella; Leptospira; Listeria; Mycobacterium; Mycoplasma; Neisseria; Pseudomonas; Rickettsia; Salmonella; Shigella; Staphylococcus; Streptococcus; Treponema; Vibrio; and Yersinia.

5. The method of claim 1, wherein the virus is selected from the group consisting of: Marburg virus, Ebola, Hantavirus, H5N1 strain of bird flu, Lassa virus, Junin virus, Dengue fever, Crimea-Congo fever virus, Bolivian hemorrhagic fever, HIV, Hep A, Hep B, Hep C, the rhino virus, polio, and chickenpox virus or Kyasanur Forest Virus (KFD).

6. A method to produce in vitro programmed immunomodulating cells comprising:
a) obtaining hematopoetic, mesenchymal, connective tissue, endothelial, cardio, induced pluripotent, osteo, muscle, and/or soft muscle tissue stem cells from a subject;
b) expanding said stem cells from a) in culture;
c) contacting said stem cells obtained in b) with a pathogen or particles thereof, RNA or DNA coding for one or more proteins from bacterial or viral origin to expressed said protein or exposing said cells obtained in b) to desired virus, bacteria or particles thereof; and
d) optionally combining said cells from step c) so as to comprise a mixture of cells.

7. The method of claim 6, wherein the hematopeitic stem cells are differentiated to antigen presenting cells.

8. The method of claim 7, wherein the antigen presenting cells are dendritic cells.

9. The method of claim 6, wherein the mixture of cells is from about 40-50% hematopoetic stem cells, about 30-40% mesenchymal stem cells, and about 10-30% connective tissue, endothelial, cardio, induced pluripotent, osteo, muscle, and/or soft muscle tissue stem cells.

10. The method of claim 6, wherein the virus is selected from the group consisting of: adenoviruses; papillomaviruses; hepadnaviruses; parvoviruses; pox viruses; Epstein-Barr virus; cytomegalovirus (CMV); herpes simplex viruses; roseolovirus; varicella zoster virus; filoviruses; paramyxoviruses; orthomyxoviruses; rhabdoviruses; arenaviruses; coronaviruses; human enteroviruses; hepatitis A virus; human rhinoviruses; polio virus; retroviruses; rotaviruses; flaviviruses; hepaciviruses; and rubella virus.

11. The method of claim 6, wherein the bacteria is selected from the group consisting of: Bacillus; Bordetella; Borrelia; Brucella; Burkholderia; Campylobacter; Chlamydia, Chlamydophila; Clostridium; Corynebacterium; Enterococcus; Escherichia; Francisella; Haemophilus; Helicobacter, Legionella; Leptospira; Listeria; Mycobacterium; Mycoplasma; Neisseria; Pseudomonas; Rickettsia; Salmonella; Shigella; Staphylococcus; Streptococcus; Treponema; Vibrio; and Yersinia.

12. The method of claim 6, wherein the virus is selected from the group consisting of: Marburg virus, Ebola, Hantavirus, H5N1 strain of bird flu, Lassa virus, Junin virus, Dengue fever, Crimea-Congo fever virus, Bolivian hemorrhagic fever, HIV, Hep A, Hep B, Hep C, the rhino virus, polio, and chickenpox virus or Kyasanur Forest Virus (KFD).

13. A method to treat or prevent a bacterial or viral infection comprising administering to a subject in need thereof an effective amount of the in vitro programmed immune stimulating cells produced by the method of claim 1.

14. The method of claim 13, wherein the administered cells are autologous to the subject.

15. The method of claim 13 wherein the cells are administered parenterally.

16. The method of claim 13, wherein the subject further undergoes hyperthermic treatment.

17. A method to treat or prevent a bacterial or viral infection comprising administering to a subject in need thereof an effective amount of the in vitro programmed immunomodulating cells produced by the method of claim 6.

18. The method of claim 17, wherein the administered cells are autologous to the subject.

19. The method of claim 17, wherein the cells are administered parenterally, such as intravenously.

20. The method of claim 17, wherein the subject further undergoes hyperthermic treatment.
